(19) United States
(12) Patent Application Publication
(10) Pub. No.: US 2008/0312085 A1
(43) Pub. Date: Dec. 18, 2008

(54) Pesticidal Compositions

(76) Inventors: Markus Kordes, Frankenthal (DE); Livio Tedeschi, Mannheim (DE); Ronan Le Vezouet, Mannheim (DE); Christopher Koradin, Ludwigshafen (DE); Deborah L. Culbertson, Fuquay Varina, NC (US)

Correspondence Address: BRINKS, HOFER, GILSON & LIONE, 2801 SLATER ROAD, SUITE 120, MORRISVILLE, NC, 27560, US

(21) Appl. No.: 12/158,261

(22) PCT Filed: Dec. 11, 2006
(86) PCT No.: PCT/EP06/69525

§ 371 Date: Jun. 19, 2008

Related U.S. Application Data
(60) Provisional application No. 60/753,367, filed on Dec. 22, 2005.

(51) Int. Cl. A01N 43/78; C07D 417/12; A01N 43/40; A01P 7/04; A01N 43/42; A01N 43/836

(52) U.S. Cl. 504/247; 546/270.7; 546/148; 544/284; 548/193; 548/152; 548/190; 548/131; 504/265; 504/266; 504/267; 504/252

(57) ABSTRACT

The present invention relates to 1-(Azolin-2-yl)-amino-alkane compounds, which are useful for combating insects, arachnids and nematodes.

The present invention also relates to a method for combating animal pests selected from insects, arachnids and nematodes, and to agricultural compositions for combating animal pests.

It has been found that animal pests can be combated by 1-(Azolin-2-yl)-amino-alkane compounds of the general formula I:

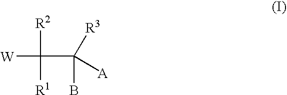

wherein
A is a radical of the formulae A¹ or A²:

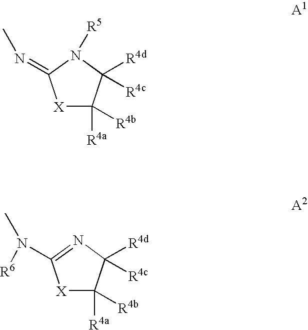

and wherein
X is sulfur or oxygen and W, B and R¹ to R⁶ are defined as in the description.

The present invention relates to 1-(Azolin-2-yl)-amino-1-phenyl-2-hetaryl-ethane compounds, which are useful for combating insects, arachnids and nematodes. The present invention also relates to a method for combating animal pests selected from insects, arachnids and nematodes, and to agricultural compositions for combating animal pests.

Animal pests and in particular insects, arachnids and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating insects, arachnids and nematodes.

Azoline compounds have been mentioned for different uses in conventional art:

JP 63203672 discloses thiazoles as anti-ulcer agents and liver disease therapeutics.

In US 2002/0010199 substituted cyclic amidine derivatives have been described as inhibitors of cell adhesion.

In GB 2121414 heterocyclic amines are employed for fungicidal purposes.

The heterocyclic imino derivatives disclosed in WO 95/33717 are used for pest control.

And in WO 2005/063724 azoline compounds with different substitution scheme are also employed for pest control.

It is therefore an object of the present invention to provide compounds having a good pesticidal activity and showing a broad activity spectrum against a large number of different animal pests, especially against difficult to control insects, arachnids and nematodes.

It has been found that these objectives can be achieved by 1-(Azolin-2-yl)-amino-1-phenyl-2-hetaryl-ethane compounds of the general formula I:

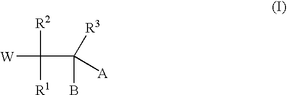

wherein
R¹, R², R³ are selected from hydrogen, C₁-C₆-alkyl, C₁-C₆-haloalkyl, C₃-C₆ cycloalkyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
phenyl or benzyl, each unsubstituted or substituted with any combination of one to five halogen, one to three C₁-C₆-alkyl, C₁-C₆-haloalkyl, C₁-C₆-alkylthio, C₁-C₆-haloalkylthio, C₁-C₆-alkoxy or C₁-C₆-haloalkoxy groups;
If A corresponds to A² and R³ is hydrogen, R¹ and R² are preferably selected dependently from one another not identically to the combination of R³ and B.
A is a radical of the formulae A¹ or A²:

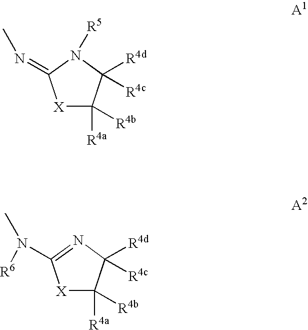

wherein
X is sulfur or oxygen;
R^{4a}, R^{4b}, R^{4c}, R^{4d} are each independently selected from one another from hydrogen, halogen, C₁-C₆-alkyl, C₁-C₆-haloalkyl, C₁-C₆-alkylamino, C₁-C₆-alkoxy, C₃-C₆-cycloalkyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
R⁵, R⁶, R⁹ are each independently selected from one another from hydrogen, cyano, nitro, formyl, C(═O)R^{5c} or C(═O)R^{6c} or C(═O)R^{9c}, C₁-C₆-alkyl, C₂-C₆-alkenyl, C₂-C₆-alkinyl, C₃-C₈-cycloalkyl, C₁-C₆-alkoxy, (C₁-C₆-alkoxy)methylen, C₁-C₆-alkylsulfinyl, C₁-C₆-alkylsulfenyl or C₁-C₆-alkylsulfonyl wherein the carbon atoms in the aliphatic radicals of the aforementioned groups may carry any combination of 1, 2 or 3 radicals, independently of one another selected from the group consisting of halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy and C₁-C₆-alkylthio;
C(O)NR^{5a}R^{5b} or C(O)NR^{6a}R^{6b} or C(O)NR^{9a}R^{9b}, (SO₂)NR^{5a}R^{5b} or (SO₂)NR^{6a}R^{6b} or (SO₂)NR^{9a}R^{9b},
phenyl, phenyloxy or benzyl, each of the last three mentioned radicals may be unsubstituted or substituted with 1 to 5 radicals, independently of one another selected from the group consisting of one to five halogen radicals, one to three C₁-C₆-alkyl, one to three C₁-C₆-haloalkyl, one to three C₁-C₆-alkylthio, one to three C₁-C₆-haloalkylthio, one to three C₁-C₆-alkoxy and one to three C₁-C₆-haloalkoxy radicals;
and wherein R^{5a}, R^{6a}, R^{9a}, R^{5b}, R^{6b}, R^{9b}, R^{5c}, R^{6c} and R^{9c} are defined as below
B is an optionally substituted phenyl ring system
W is 5- to 6-membered heteroaromatic ring which may contain 1 to 4 heteroatoms selected from oxygen, nitrogen and sulfur,
   wherein the heteroaromatic ring may optionally be fused to a ring selected from phenyl and a 5- to 6-membered saturated, partially unsaturated or aromatic heterocyclic ring which may contain 1 to 3 heteroatoms selected from oxygen, nitrogen and sulfur, and wherein the 5- to 6-membered heteroaromatic ring or the respective fused ring systems may be unsubstituted or optionally substituted by R⁹ and/or any combination of 1 to 4 groups R⁸:

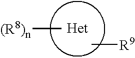

wherein n is 0, 1, 2, 3, or 4;
and R⁸ is defined as below;
and R⁹ is defined as above
and wherein
R⁸ is selected from halogen, OH, SH, NH₂, SO₃H, COOH, cyano, azido, nitro, formyl, CONH₂, CSNH₂, CH═N—OH, CH═N—O—(C₁-C₆)-alkyl, C(═O)R^{8c}, C(═S)R^{8c}, C₁-C₆-alkyl, C₂-C₆-alkenyl, C₂-C₆-alkynyl, C₃-C₈-cycloalkyl, C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkylthio, C₂-C₆-alkenylthio, C₂-C₆-alkynylthio, C₁-C₆-alkylsulfonyl, C₂-C₆-alkenylsulfonyl, C₂-C₆-alkynylsulfonyl, (C₁-C₆-alkyl)carbonyl, (C₂-C₆-alkenyl)-carbonyl, (C₂-C₆-alkynyl)-carbonyl, C₁-C₆-alkoxy, C₂-C₆-alkenyloxy, C₂-C₆-alkynyloxy, (C₁-C₆-alkoxy)carbonyl, (C₂-C₆-alkenyloxy)carbonyl, (C₂-C₆-alkynyloxy)-carbonyl, (C₁-C₆-alkyl)carbonyloxy, (C₂-C₆-alkenyl-)carbonyl-oxy, (C₂-C₆-alkynyl-)carbonyloxy, (C₁-C₆-alkyl)carbonyl-amino, (C₂-C₆-alkenyl)carbonyl-amino, (C₂-C₆-alkynyl)carbonyl-amino, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
C(O)NR^{8a}R^{8b}, (SO₂)NR^{8a}R^{8b}; wherein R^{8a}, R^{8b} and R^{8c} are defined as below,
a radical Y—Ar or a radical Y-Cy, wherein
Y is a single bond, O, S, NH, C₁-C₆-alkandiyl or C₁-C₆-alkandiyloxy;
Ar is phenyl, naphthyl or a mono- or bicyclic 5- to 10-membered heteroaromatic ring, which contains 1, 2, 3 or 4 heteroatoms selected from 1 or 2 oxygen, 1 or 2 sulfur and 1 to 3 nitrogen atoms as ring members, wherein Ar is unsubstituted or may carry any combination of one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
Cy is C₃-C₁₂-cycloalkyl, which is unsubstituted or substituted with one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
and wherein
R^{5a}, R^{6a}, R^{8a} and R^{5b}, R^{6b}, R^{8b}
are each independently selected from one another from hydrogen, C₁-C₆-alkyl, C₁-C₆-haloalkyl, C₂-C₆-alkenyl, or C₂-C₆-alkynyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
and R^{5c}, R^{6c}, R^{8c} and R^{9c}
are selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl and heteroaryl, which can be a mono- or bicyclic 5 to 10 membered heteroaromatic ring, which contains 1, 2, 3 or 4 heteroatoms selected from O, S and N
or the enantiomers, diastereomers or salts thereof.

Preferred are 1-(Azolin-2-yl)-amino-1-phenyl-2-hetaryl-ethane compound wherein B is selected from:

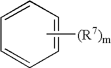

and wherein
m is 0, 1, 2, 3, 4 or 5;
and R⁷ is selected from halogen, OH, SH, NH₂, SO₃H, COOH, cyano, azido, nitro, formyl, CONH₂, CSNH₂, CH═N—OH, CH═N—O—(C₁-C₆)-alkyl, C(═O)R^{7c}, C(═S)R^{7c}, C₁-C₆-alkyl, C₂-C₆-alkenyl, C₂-C₆-alkynyl, C₃-C₈-cycloalkyl, C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkylthio, C₂-C₆-alkenylthio, C₂-C₆-alkynylthio, C₁-C₆-alkylsulfonyl, C₂-C₆-alkenylsulfonyl, C₂-C₆-alkynylsulfonyl, (C₁-C₆-alkyl)carbonyl, (C₂-C₆-alkenyl)-carbonyl, (C₂-C₆-alkynyl)-carbonyl, C₁-C₆-alkoxy, C₂-C₆-alkenyloxy, C₂-C₆-alkynyloxy, (C₁-C₆-alkoxy)carbonyl, (C₂-C₆-alkenyloxy)carbonyl, (C₂-C₆-alkynyloxy)-carbonyl, (C₁-C₆-alkyl)carbonyloxy, (C₂-C₆-alkenyl-)carbonyl-oxy, (C₂-C₆-alkynyl-)carbonyloxy, (C₁-C₆-alkyl)carbonyl-amino, (C₂-C₆-alkenyl)carbonyl-amino, (C₂-C₆-alkynyl)carbonyl-amino, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio; C(O)NR^{7a}R^{7b} or (SO₂)NR^{7a}R^{7b}; wherein R^{7a}, R^{7b} and R^{7c} are defined as below, a radical Y—Ar or a radical Y-Cy, wherein
Y is a single bond, O, S, NH, C₁-C₆-alkandiyl or C₁-C₆-alkandiyloxy;
Ar is phenyl, naphthyl or a mono- or bicyclic 5- to 10-membered heteroaromatic ring, which contains 1, 2, 3 or 4 heteroatoms selected from 1 or 2 oxygen, 1 or 2 sulfur and 1 to 3 nitrogen atoms as ring members, wherein Ar is unsubstituted or may carry any combination of one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
Cy is C₃-C₁₂-cycloalkyl, which is unsubstituted or substituted with one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
   and wherein the radical R⁷ that is bound to adjacent carbon atoms of the phenyl rings may optionally form, together with said carbon atoms, a fused benzene ring, a fused saturated or partially unsaturated 5-, 6-, or 7-membered carbocycle or a fused 5-, 6- or 7-membered heterocycle, which contains 1, 2, 3 or 4 heteroatoms selected from 1 or 2 oxygen, 1 or 2 sulfur and 1 to 3 nitrogen atoms as ring members, and wherein the fused ring is unsubstituted or may carry 1, 2, 3 or 4 radicals, independently of one another selected from the group consisting of halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy and C₁-C₆-alkylthio;
   and wherein R^{7a} and R^{7b} are each independently selected from one another from hydrogen, C₁-C₆-alkyl, C₁-C₆-haloalkyl, C₂-C₆-alkenyl, or C₂-C₆-alkynyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
   and wherein R^{7c} are selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl and heteroaryl, which can be a mono- or bicyclic 5 to 10 membered heteroaromatic ring, which contains 1, 2, 3 or 4 heteroatoms selected from O, S and N.

Therefore, the present invention relates to 1-(Azolin-2-yl)-amino-1-phenyl-2-hetaryl-ethane compounds of the general formula I and to the agriculturally acceptable salts thereof. These compounds have a high pesticidal activity and are active against a broad spectrum of animal pests selected from insects, arachnids and nematodes. Therefore the invention relates also to a method of combating animal pests, selected from insects, arachnids and nematodes, which comprises contacting the animal pests, their habit, breeding ground, food supply, plant, seed, soil, area, material or environment in which the animal pests are growing or may grow, or the materials, plants, seeds, soils, surfaces or spaces to be protected from 1 attack or infestation by insects, arachnids or nematodes with a pesticidally effective amount of at least one 1-(Azolin-2-yl)-amino-1-phenyl-2-hetaryl-ethane compounds of the general formula I of the general formula I and/or at least one agriculturally acceptable salt thereof.

Furthermore, the present invention provides a method for protecting crops from attack or infestation by insects, arachnids or nematodes, which comprises contacting a crop with a pesticidally effective amount of a 1-(Azolin-2-yl)-amino-1-phenyl-2-hetaryl-ethane compounds of the general formula I and/or at least one salt thereof.

Furthermore, the invention relates to agricultural compositions, preferably in the form of directly sprayable solutions, emulsions, pastes oil dispersions, powders, materials for scattering, dusts or in the form of granules, which comprise at least one 1-(Azolin-2-yl)-amino-1-phenyl-2-hetaryl-ethane compound of the general formula I as defined above or a salt thereof, admixed with one or more agronomically acceptable inert, solid or liquid carrier(s) and, if desired, at least one surfactant.

The compounds of the formula I may have one or more centers of chirality, in which case they are present as mixtures of stereoisomers, such as enantiomers or diastereomers. The present invention provides both the pure stereoisomers, e.g. the pure enantiomers or diastereomers, and mixtures thereof. The compounds of the formula I may also exist in the form of different tautomers. The invention comprises the single tautomers, if separable, as well as the tautomer mixtures.

Salts of the compounds of the formula I which are suitable for the use according to the invention are especially agriculturally acceptable salts. They can be formed in a customary method, e.g. by reacting the compound with an acid of the anion in question if the compound of formula I has a basic functionality or by reacting an acidic compound of formula I with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄+) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl and/or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

Examples of other meanings are:

The term “C₁-C₆-alkyl” as used herein and in the alkyl moieties of C₁-C₆-alkoxy, C₁-C₆-alkylamino, di(C₁-C₆-alkyl)amino, C₁-C₆-alkylthio, C₁-C₆-alkylsulfonyl, C₁-C₆-alkylsulfoxyl, C₁-C₆-alkylcarbonyl, C₁-C₆-alkoxycarbonyl, C₁-C₆-alkylthiocarbonyl, and C₁-C₆-alkylcarbonyloxy refer to a saturated straight-chain or branched hydrocarbon group having 1 to 6 carbon atoms, especially 1 to 4 carbon groups, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “C₁-C₆-haloalkyl” as used herein refers to a straight-chain or branched saturated alkyl group having 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term, “C₁-C₆-alkoxy” as used herein refers to a straight-chain or branched saturated alkyl group having 1 to 6 carbon atoms (as mentioned above) which is attached via an oxygen atom. Examples include C₁-C₆-alkoxy such as methoxy, ethoxy, OCH₂—C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethyl butoxy, 3,3-dimethylbutoxy, 1-ethyl butoxy, 2-ethyl butoxy, 1,1,2-trimethylpropoxy, 1,2,2-trimethylpropoxy, 1-ethyl-1-methylpropoxy, 1-ethyl-2-methylpropoxy and the like.

The term “C₁-C₆-haloalkoxy” as used herein refers to a C₁-C₆-alkoxy group as mentioned above wherein the hydrogen atoms are partially or fully substituted by fluorine, chlorine, bromine and/or iodine, i.e., for example, C₁-C₆-haloalkoxy such as chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nonafluorobutoxy, 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy or dodecafluorohexoxy, in particular chloromethoxy, fluoromethoxy, difluoromethoxy, trifluoromethoxy, 2-fluoroethoxy, 2-chloroethoxy or 2,2,2-trifluoroethoxy.

The term “C₁-C₆-alkoxy-C₁-C₆-alkyl” as used herein refers to C₁-C₆-alkyl wherein 1 carbon atom carries a C₁-C₆-alkoxy radical as mentioned above. Examples are CH₂—OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl, 4-(1,1-dimethylethoxy)butyl and the like.

The term “(C₁-C₆-alkyl)carbonyl” as used herein refers to a straight-chain or branched saturated alkyl group having 1 to 6 carbon atoms (as mentioned above) bonded via the carbon atom of the carbonyl group at any bond in the alkyl group. Examples include C₁-C₆-alkylcarbonyl such CO—CH₃, CO—C₂H₅, n-propylcarbonyl, 1-methylethylcarbonyl, n-butylcarbonyl, 1-methylpropylcarbonyl, 2-methylpropylcarbonyl, 1,1-dimethylethylcarbonyl, n-pentylcarbonyl, 1-methylbutylcarbonyl, 2-methylbutylcarbonyl, 3-methylbutylcarbonyl, 1,1-dimethylpropylcarbonyl, 1,2-dimethylpropylcarbonyl, 2,2-dimethylpropylcarbonyl, 1-ethylpropylcarbonyl, n-hexylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl and the like.

The term “(C₁-C₆-alkoxy)carbonyl” as used herein refers to a straight-chain or branched alkoxy group (as mentioned above) having 1 to 6 carbon atoms attached via the carbon atom of the carbonyl group, for example CO—OCH₃, CO—OC₂H₅, COO—CH₂—C₂H₅, CO—OCH(CH₃)₂, n-butoxycarbonyl, CO—OCH(CH₃)—C₂H₅, CO—OCH₂—CH(CH₃)₂, CO—OC(CH₃)₃, n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methyl pentoxycarbonyl, 1,1-dimethyl butoxycarbonyl, 1,2-dimethylbutoxycarbonyl, 1,3-dimethyl butoxycarbonyl, 2,2-dimethyl butoxycarbonyl, 2,3-dimethyl butoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “(C₁-C₆-alkyl)carbonyloxy” as used herein refers to a straight-chain or branched saturated alkyl group having 1 to 6 carbon atoms (as mentioned above) bonded via the carbon atom of the carbonyloxy group at any bond in the alkyl group, for example O—CO—CH₃, O—CO—C₂H₅, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy or 1,2-dimethylpropylcarbonyloxy.

The term “C₁-C₆-alkylthio (C₁-C₆-alkylsulfanyl: C₁-C₆-alkyl-S—)” as used herein refers to a straight-chain or branched saturated alkyl group having 1 to 6 carbon atoms (as mentioned above) which is attached via a sulfur atom, for example C₁-C₄-alkylthio such as methylthio, ethylthio, propylthio, 1-methylethylthio, butylthio, 1-methylpropylthio, 2-methylpropylthio, 1,1-dimethylethylthio, n-pentylthiocarbonyl, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutlthio, 2-ethylbutylthio, 1,1,2-trimethylpropylthio, 1,2,2-trimethylpropylthio, 1-ethyl-1-methylpropylthio or 1-ethyl-2-methylpropylthio.

The term “(C₁-C₆-alkylthio)carbonyl” as used herein refers to a straight-chain or branched alkthio group (as mentioned above) having 1 to 6 carbon atoms attached via the carbon atom of the carbonyl group. Examples include CO—SCH₃, CO—SC₂H₅, CO—SCH₂—C₂H₅, CO—SCH(CH₃)₂, n-butylthiocarbonyl, CO—SCH(CH₃)—C₂H₅, CO—SCH₂—CH(CH₃)₂, CO—SC(CH₃)₃, n-pentylthiocarbonyl, 1-methylbutylthiocarbonyl, 2-methylbutylthiocarbonyl, 3-methylbutylthiocarbonyl, 2,2-dimethylpropylthiocarbonyl, 1-ethylpropylthiocarbonyl, n-hexylthiocarbonyl, 1,1-dimethylpropylthiocarbonyl, 1,2-dimethylpropylthiocarbonyl, 1-methylpentylthiocarbonyl, 2-methylpentylthiocarbonyl, 3-methylpentylthiocarbonyl, 4-methylpentylthiocarbonyl, 1,1-dimethylbutylthiocarbonyl, 1,2-dimethylbutylthiocarbonyl, 1,3-dimethylbutylhiocarbonyl, 2,2-dimethylbutylthiocarbonyl, 2,3-dimethylbutylthiocarbonyl, 3,3-dimethylbutylthiocarbonyl, 1-ethylbutlthioycarbonyl, 2-ethylbutylthiocarbonyl, 1,1,2-trimethylpropylthiocarbonyl, 1,2,2-trimethylpropylthiocarbonyl, 1-ethyl-1-methylpropylthiocarbonyl or 1-ethyl-2-methylpropylthiocarbonyl.

The term “C₁-C₆-alkylsulfinyl” (C₁-C₆-alkylsulfoxyl: C₁-C₆-alkyl-S(═O)—), as used herein refers to a straight-chain or branched saturated hydrocarbon group (as mentioned above) having 1 to 6 carbon atoms bonded through the sulfur atom of the sulfinyl group at any bond in the alkyl group, for example SO—CH₃, SO—C₂H₅, n-propylsulfinyl, 1-methylethylsulfinyl, n-butylsulfinyl, 1-methylpropylsulfinyl, 2-methylpropylsulfinyl, 1,1-dimethylethylsulfinyl, n-pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethyl propylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The term “C₁-C₆-alkylamino” refers to a secondary amino group carrying one alkyl group as defined above, e.g. methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino.

The term “di(C₁-C₆-alkyl)amino” refers to a tertiary amino group carrying two alkyl radicals as defined above, e.g. dimethylamino, diethylamino, di-n-propylamino, diisopropylamino, N-ethyl-N-methylamino, N-(n-propyl)-N-methylamino, N-(isopropyl)-N-methylamino, N-(n-butyl)-N-methylamino, N-(n-pentyl)-N-methylamino, N-(2-butyl)-N-methylamino, N-(isobutyl)-N-methylamino, N-(n-pentyl)-N-methylamino, N-(n-propyl)-N-ethylamino, N-(isopropyl)-N-ethylamino, N-(n-butyl)-N-ethylamino, N-(n-pentyl)-N-ethylamino, N-(2-butyl)-N-ethylamino, N-(isobutyl)-N-ethylamino or N-(n-pentyl)-N-ethylamino.

The term “C₁-C₆-alkylsulfonyl” (C₁-C₆-alkyl-S(═O)₂—) as used herein refers to a straight-chain or branched saturated alkyl group having 1 to 6 carbon atoms (as mentioned above) which is bonded via the sulfur atom of the sulfonyl group at any bond in the alkyl group, for example SO₂—CH₃, SO₂—C₂H₅, n-propylsulfonyl, SO₂—CH(CH₃)₂, n-butylsulfonyl, 1-methylpropylsulfonyl, 2-methylpropylsulfonyl, SO₂—C(CH₃)₃, n-pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, n-hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “C₂-C₆-alkenyl” as used herein and in the alkenyl moieties of C₂-C₆-alkenyloxy, C₂-C₆-alkenylamino, C₂-C₆-alkenylthio, C₂-C₆-alkenylsulfonyl, (C₂-C₆-alkenyl)carbonyl, (C₂-C₆-alkenyloxy)carbonyl and (C₂-C₆-alkenyl)carbonyloxy refers to a straight-chain or branched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term, “C₂-C₆-alkenyloxy” as used herein refers to a straight-chain or branched saturated alkenyl group having 2 to 6 carbon atoms (as mentioned above) which is attached via an oxygen atom, such as vinyloxy, allyloxy (propen-3-yloxy), methallyloxy, buten-4-yloxy, etc.

The term “C₂-C₆-alkenylthio” as used herein refers to a straight-chain or branched saturated alkenyl group having 2 to 6 carbon atoms (as mentioned above) which is attached via a sulfur atom, for example vinylsulfanyl, allylsulfanyl (propen-3-ylthio), methallylsufanyl, buten-4-ylsulfanyl, etc.

The term “C₂-C₆-alkenylamino” as used herein refers to a straight-chain or branched saturated alkenyl group having 2 to 6 carbon atoms (as mentioned above) which is attached via a sulfur atom, for example vinylamino, allylamino (propen-3-ylamino), methallylamino, buten-4-ylamino, etc.

The term “C₂-C₆-alkenylsulfonyl” as used herein refers to a straight-chain or branched saturated alkenyl group having 2 to 6 carbon atoms (as mentioned above) which is attached via a sulfonyl (SO₂) group, for example vinylsulfonyl, allylsulfonyl (propen-3-ylsulfonyl), methallylsulfonyl, buten-4-ylsulfonyl, etc.

The term “C₂-C₆-alkynyl” as used herein and in the alkynyl moieties of C₂-C₆-alkynyloxy, C₂-C₆-alkynylamino, C₂-C₆-alkynylthio, C₂-C₆-alkynylsulfonyl, C₂-C₆-alkynylcarbonyl, C₂-C₆-alkynyloxycarbonyl and C₁-C₆-alkynylcarbonyloxy refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methyl pent-1-yn-1-yl, 3-methyl pent-1-yn-3-yl, 3-methyl pent-1-yn-4-yl, 3-methyl pent-1-yn-5-yl, 4-methyl pent-1-yn-1-yl, 4-methyl pent-2-yn-4-yl or 4-methyl pent-2-yn-5-yl and the like.

The term, “C₂-C₆-alkynyloxy” as used herein refers to a straight-chain or branched saturated alkynyl group having 2 to 6 carbon atoms (as mentioned above) which is attached via an oxygen atom, such as propargyloxy (propin-3-yloxy), butin-3-yloxy, and butin-4-yloxy.

The term “C₂-C₆-alkynylthio” as used herein refers to a straight-chain or branched saturated alkynyl group having 2 to 6 carbon atoms (as mentioned above) which is attached via a sulfur atom, such as propargylsulfanyl (propin-3-ylthio), butin-3-ylsufanyl and butin-4-ylsulfanyl.

The term “C₂-C₆-alkynylamino” as used herein refers to a straight-chain or branched saturated alkynyl group having 2 to 6 carbon atoms (as mentioned above) which is attached via a sulfur atom, such as propargylamino (propin-3-ylamino), butin-3-amino, and butin-4-ylamino.

The term “C₂-C₆-alkynylsulfonyl” as used herein refers to a straight-chain or branched saturated alkynyl group having 2 to 6 carbon atoms (as mentioned above) which is attached via a sulfonyl (SO₂) group, such as propargylsulfonyl (propin-3-yltsulfonyl), butin-3-ylsulfonyl and butin-4-ylsulfonyl.

The term “C₃-C₁₂-cycloalkyl” as used herein refers to a mono- or bi- or polycyclic hydrocarbon radical having 3 to 12 carbon atoms, in particular 3 to 6 carbon atoms. Examples of monocyclic radicals comprise cyclopropyl, cyclobutyl, cyclopentyl, cyclohexyl, cycloheptyl, cyclooctyl, cyclononyl and cyclodecyl. Examples of bicyclic radicals comprise bicyclo[2.2.1]heptyl, bicyclo[3.1.1]heptyl, bicyclo[2.2.2]octyl and bicyclo[3.2.1]nonyl. Examples of tricylcic radicals are adamantyl and homoadamantyl.

The term “mono- or bicyclic heteroaromatic ring” as used herein refers to a monocyclic heteroaromatic radical which has 5 or 6 ring members, which may comprise a fused 5, 6 or 7 membered ring thus having a total number of ring members from 8 to 10, wherein in each case 1, 2, 3 or 4 of these ring members are heteroatoms selected, independently from each other, from the group consisting of oxygen, nitrogen and sulfur. The heterocyclic radical may be attached to the remainder of the molecule via a carbon ring member or via a nitrogen ring member. The fused ring comprises C₅-C₇-cycloalkyl, C₅-C₇-cycloalkenyl, or 5 to 7 membered heterocyclyl and phenyl.

Examples for monocyclic 5- to 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

Examples for 5- to 6-membered heteroaromatic rings carrying a fused phenyl ring are quinolinyl, isoquinolinyl, indolyl, indolizinyl, isoindolyl, indazolyl, benzofuryl, benzthienyl, benzo[b]thiazolyl, benzoxazolyl, benzthiazolyl, benzoxazolyl, and benzimidazolyl. Examples for 5- to 6-membered heteroaromatic rings carrying a fused cycloalkenyl ring are dihydroindolyl, dihydroindolizinyl, dihydroisoindolyl, dihydrochinolinyl, dihydroisochinolinyl, chromenyl, chromanyl and the like.

The term “5 to 7 membered heterocyclyl” comprises monocyclic heteroaromatic rings as defined above and nonaromatic saturated or partially unsaturated heterocyclic rings having 5, 6 or 7 ring members. Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, dioxanyl, thiopyranyl, dihydrothiopyranyl, tetrahydrothiopyranyl, morpholinyl, thiazinyl and the like.

As regards the pesticidal activity of the compounds of formula I, preference is given to those 1-(Azolin-2-yl)-amino-1-phenyl-2-hetaryl-ethane compounds of formula I, in which the variables—independently of one another or in combination with any of the other variables—have the following meanings:

Preferred are 1-(Azolin-2-yl)-amino-1-phenyl-2-hetaryl-ethane compounds, wherein R¹, R² and R³ are selected from hydrogen, C₁-C₆-alkyl, C₁-C₆-haloalkyl, C₃-C₆-cycloalkyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio; If A corresponds to A² and R³ is hydrogen, R¹ and R² are preferably selected dependently from one another not identically to the combination of R³ and B.

More preferred are (Azolin-2-yl)-amino-1-phenyl-2-hetaryl-ethane compounds, wherein R¹, R² and R³ are selected from hydrogen and C₁-C₆-alkyl, especially methyl, ethyl, n-propyl, iso-propyl, n-butyl and iso-butyl.

Most preferred are 1-(Azolin-2-yl)-amino-1-phenyl-2-hetaryl-ethane compound, wherein R¹, R² and R³ are selected from hydrogen.

Preferred are 1-(Azolin-2-yl)-amino-1-phenyl-2-hetaryl-ethane compounds according to claim 1, wherein R^{4a}, R^{4b}, R^{4c}, R^{4d} are each independently selected from one another from hydrogen, halogen, C₁-C₆-alkyl and C₁-C₆-haloalkyl

More preferred are 1-(Azolin-2-yl)-amino-1-phenyl-2-hetaryl-ethane compounds according to claim 1, wherein R^{4a}, R^{4b}, R^{4c} and R^{4d} are selected from hydrogen.

Preferred are 1-(Azolin-2-yl)-amino-1-phenyl-2-hetaryl-ethane compounds according to claim 1, wherein R⁵, R⁶, R⁹ are each independently selected from one another from hydrogen, cyano, nitro, formyl, C(═O)R^{5c}, C(═O)R^{6c}, C(═O)R^{9c}, C₁-C₆-alkyl, C₂-C₆-alkinyl, C₃-C₈-cycloalkyl, (C₁-C₆-alkoxy)methylen, C₁-C₆-alkylsulfinyl, C₁-C₆-alkylsulfenyl or C₁-C₆-alkylsulfonyl wherein the carbon atoms in the aliphatic radicals of the aforementioned groups may carry any combination of 1, 2 or 3 radicals, independently of one another selected from the group consisting of halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy and C₁-C₆-alkylthio and wherein R^{5c}, R^{6c} and R^{9c} are selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl and heteroaryl, which can be a mono- or bicyclic 5 to 10 membered heteroaromatic ring, which contains 1,2, 3 or 4 heteroatoms selected from O, S and N.

More preferred are 1-(Azolin-2-yl)-amino-1-phenyl-2-hetaryl-ethane compounds according to claim 1, wherein R⁵, R⁶, R⁹ are each independently selected from one another from hydrogen, cyano, nitro, formyl, C(═O)R^{5c} or C(═O)R^{6c} or C(═O)R^{9c} and C₁-C₆-alkyl and wherein R^{5c}, R^{6c} and R^{9c} are selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl and heteroaryl, which can be a mono- or bicyclic 5 to 10 membered heteroaromatic ring, which contains 1,2, 3 or 4 heteroatoms selected from O, S and N.

Preferred are 1-(Azolin-2-yl)-amino-1-phenyl-2-hetaryl-ethane compounds of formula I wherein B is selected from:

### a) Phenyl:

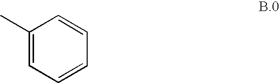

b) Monosubstituted phenyl radicals:

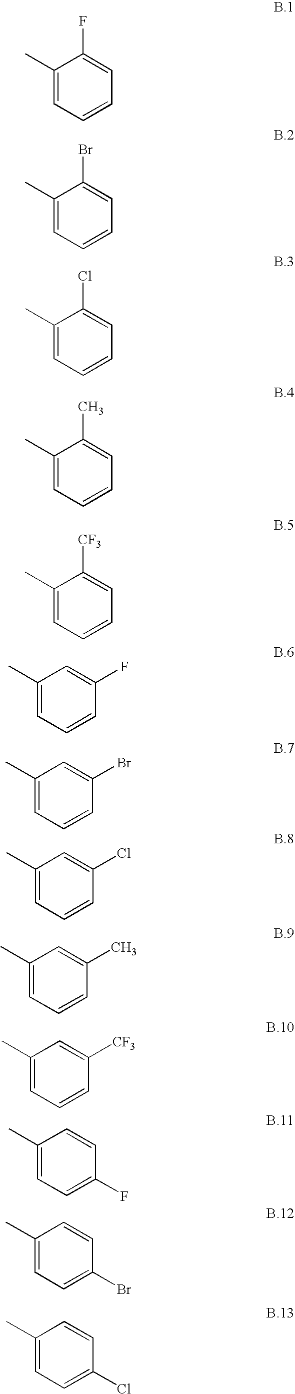

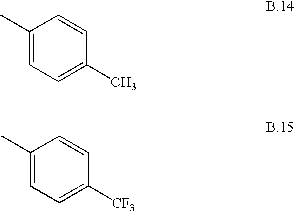

c) disubstituted phenyl radicals:

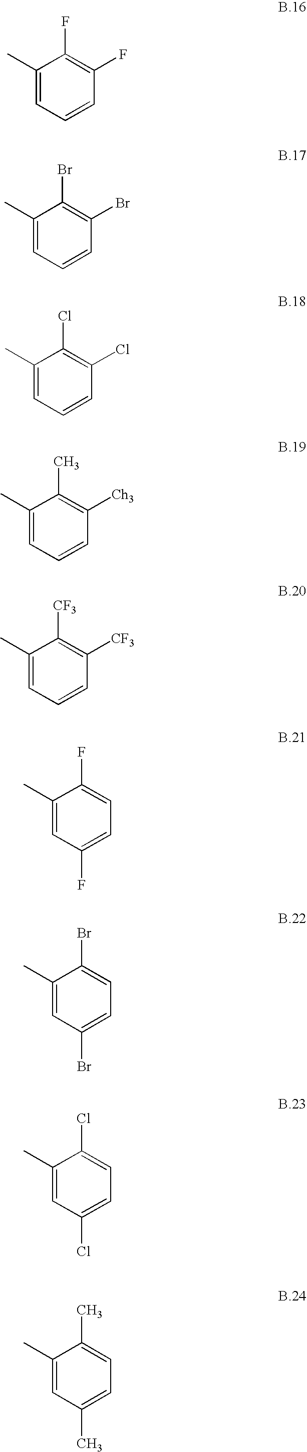

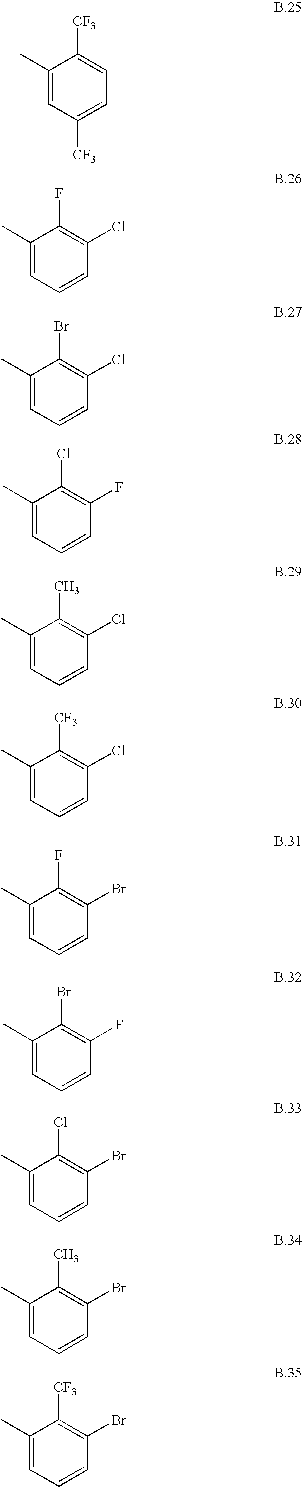

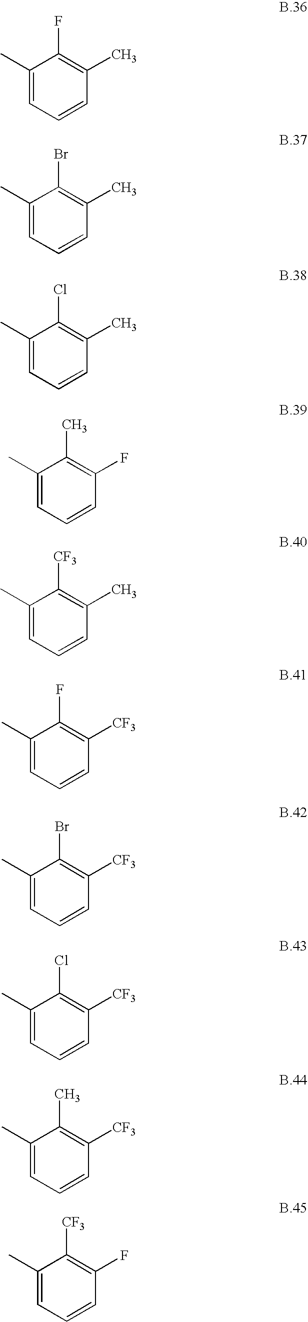

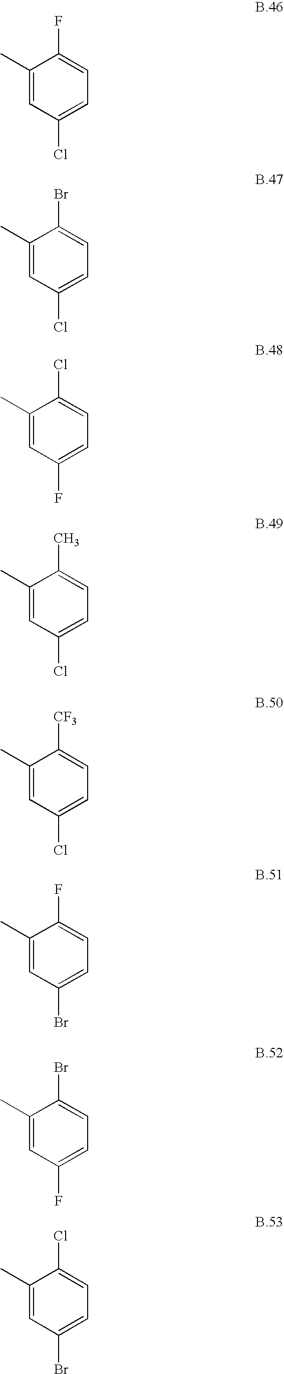

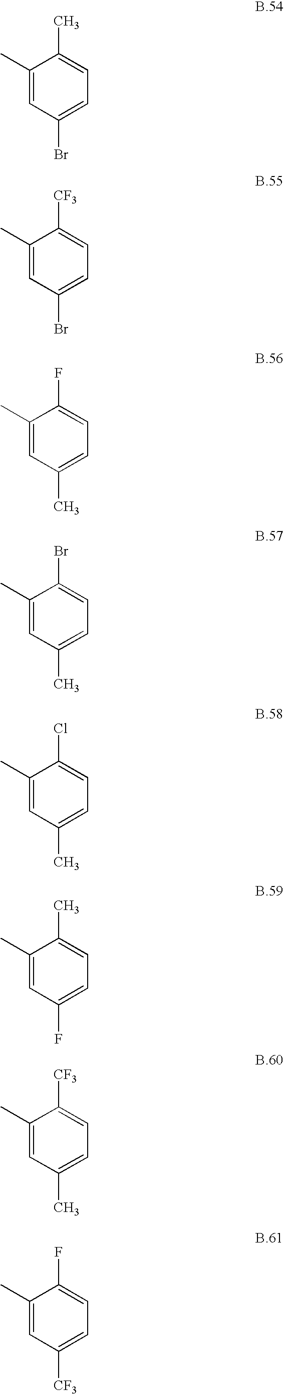

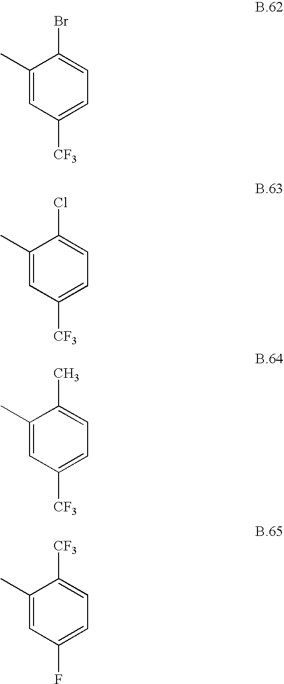

d) and trisubstituted phenyl radicals:

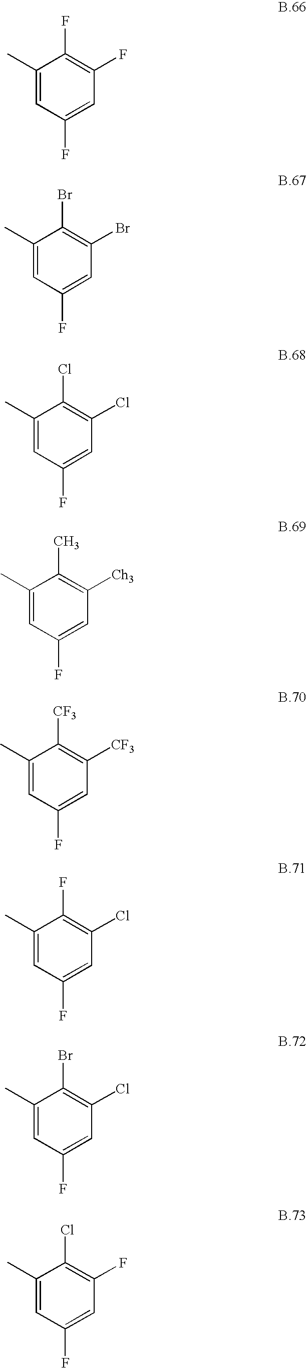

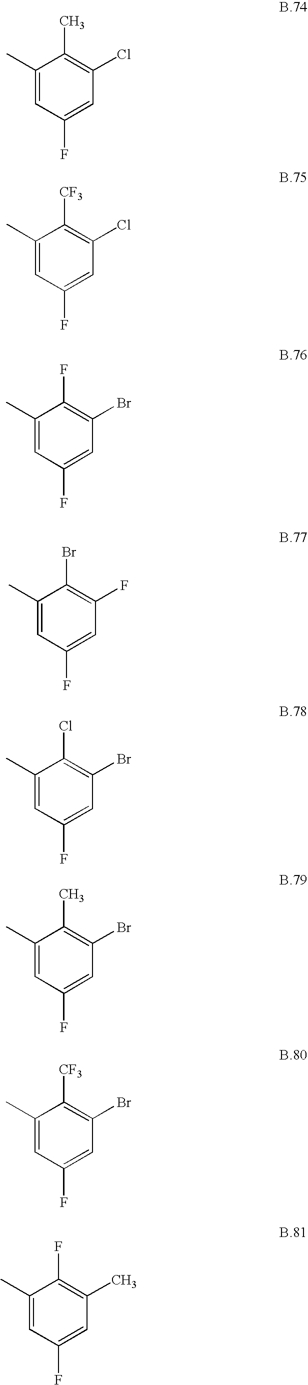

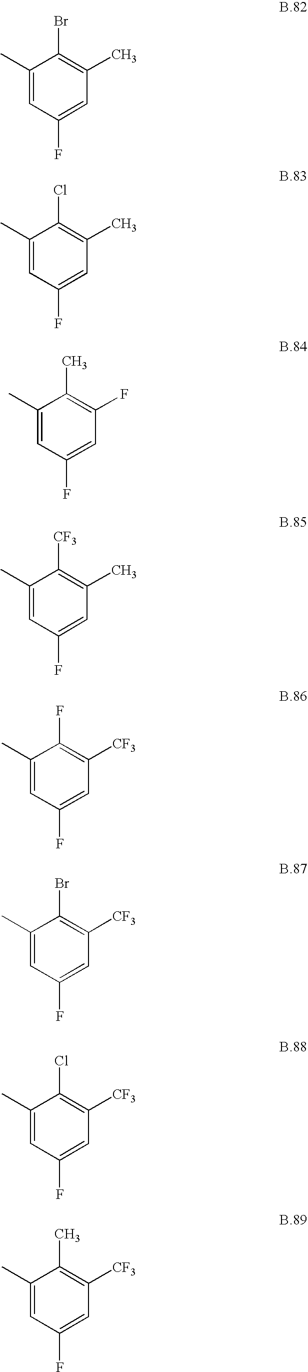

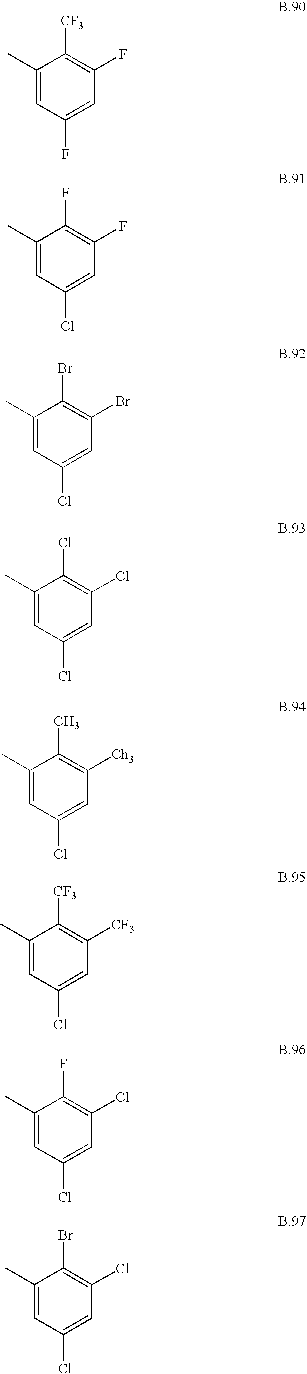

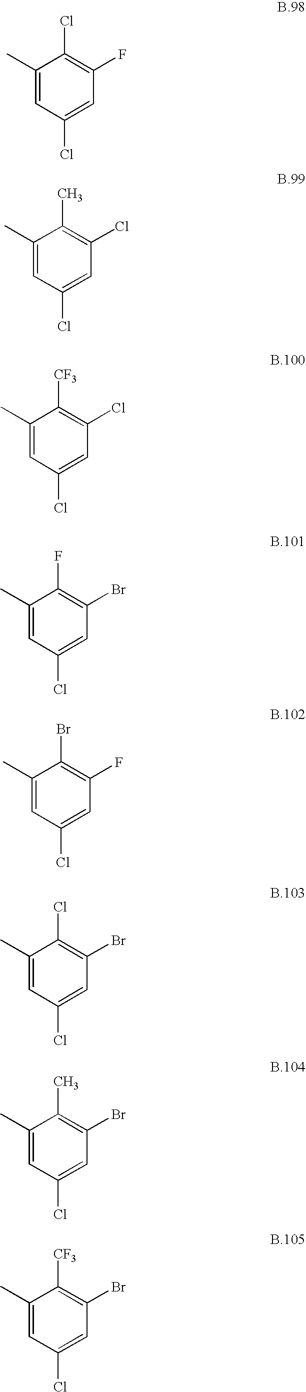

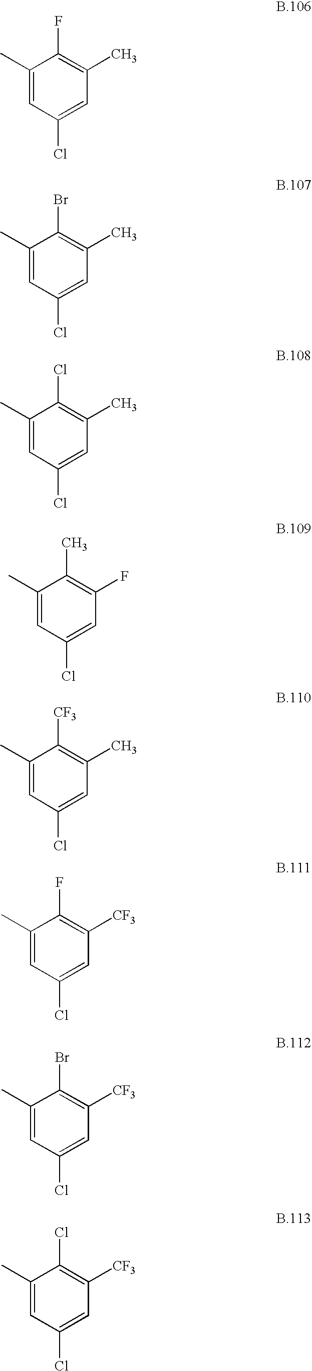

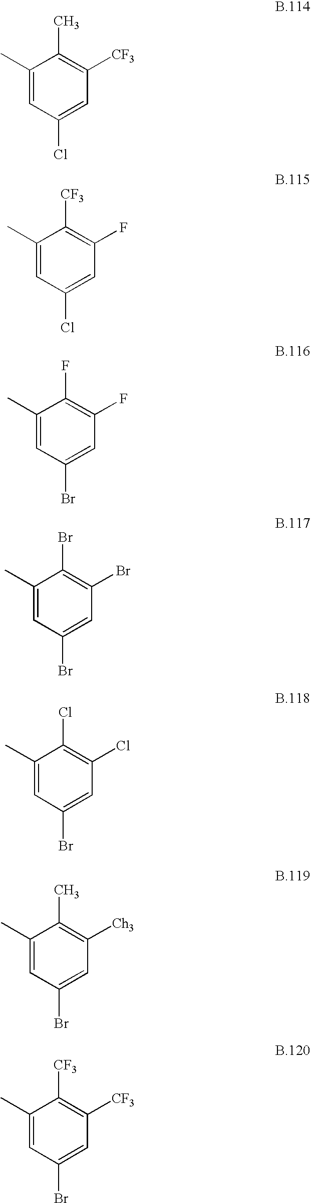

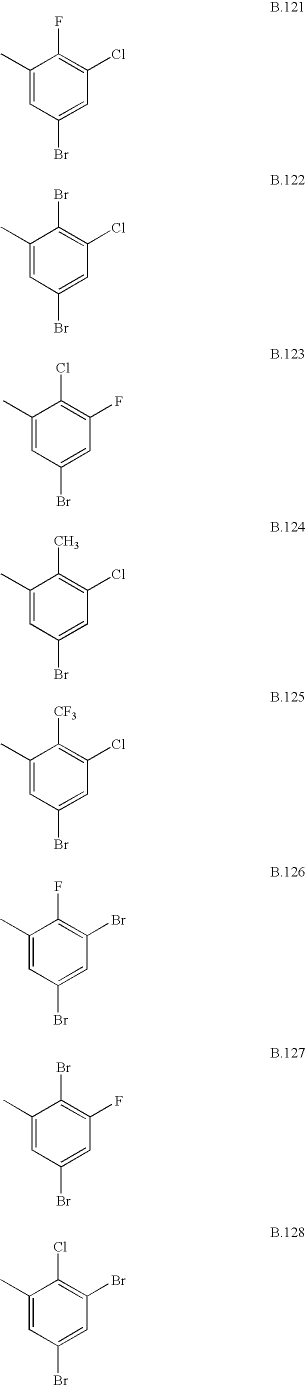

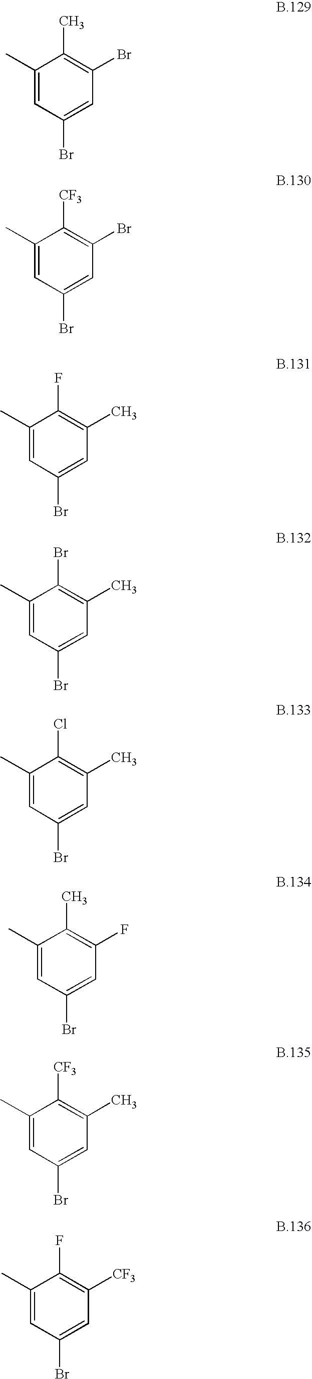

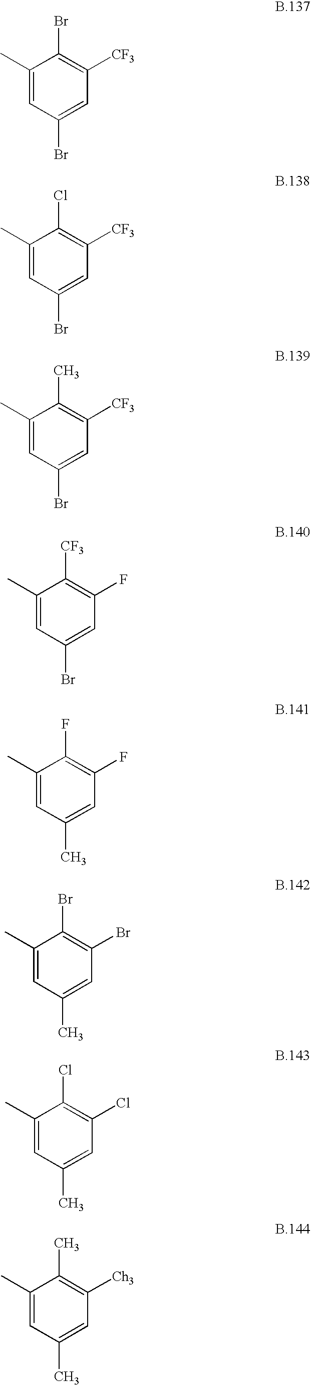

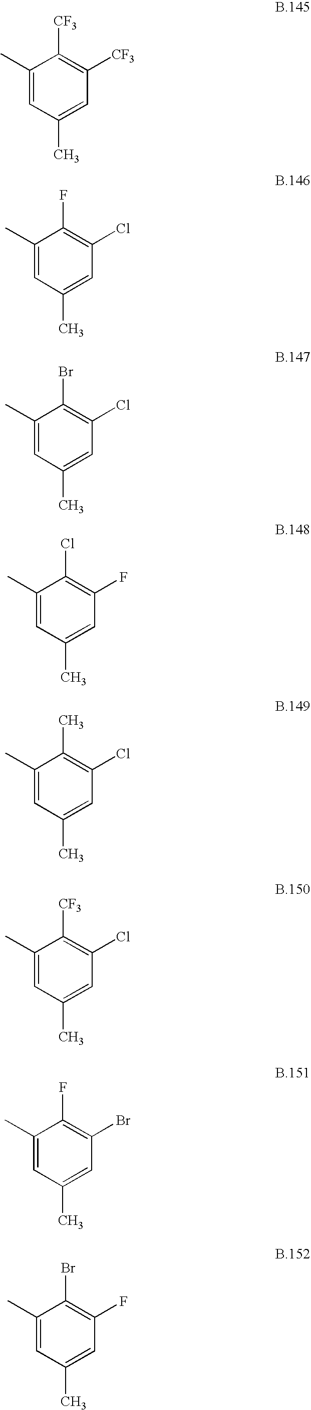

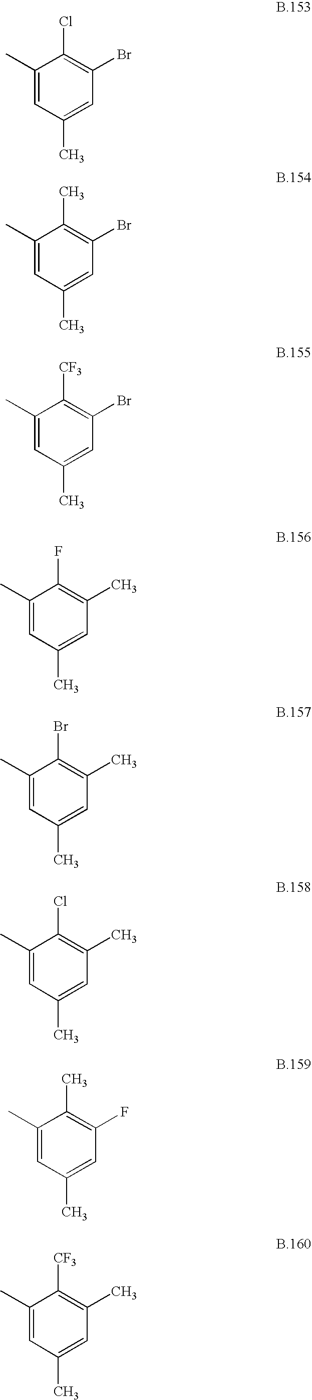

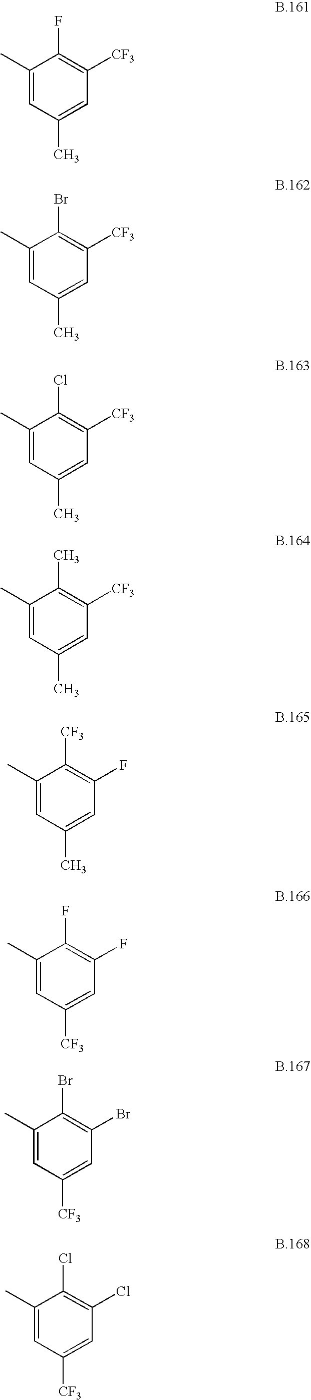

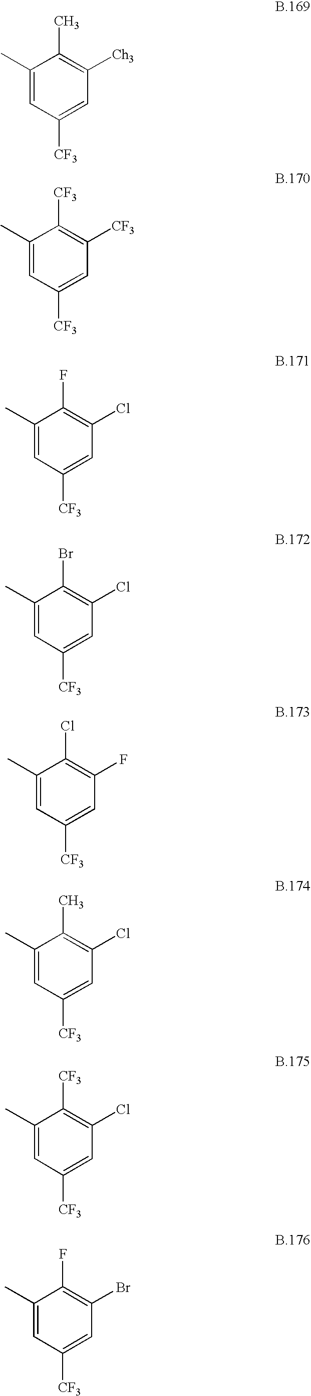

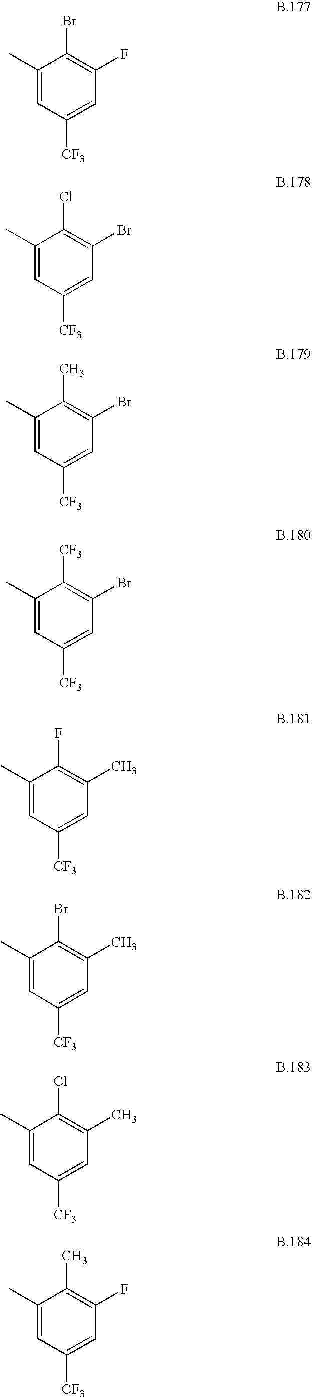

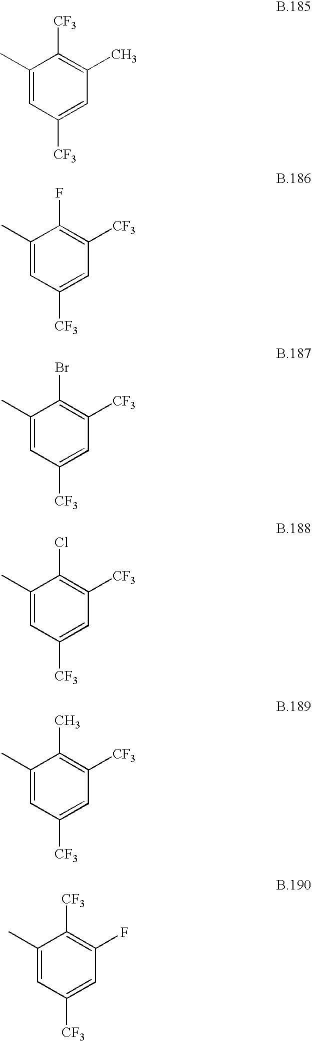

And wherein the free bond denotes the position of attachment in formula I

Preferred are 1-(Azolin-2-yl)-amino-1-phenyl-2-hetaryl-ethane compounds of formula I wherein W is selected from:

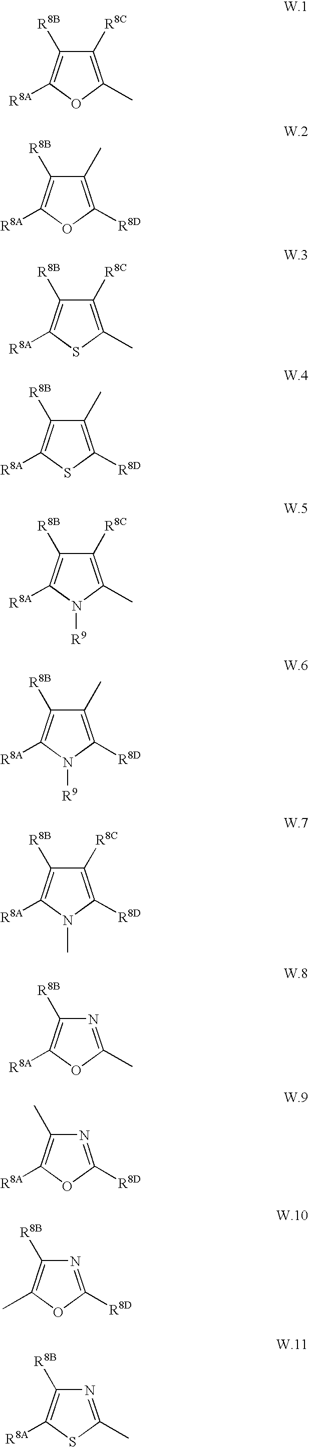

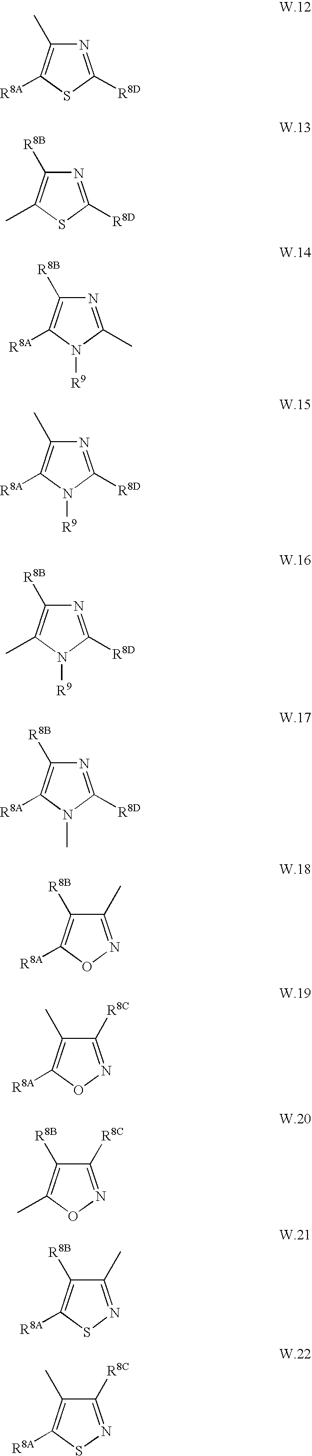

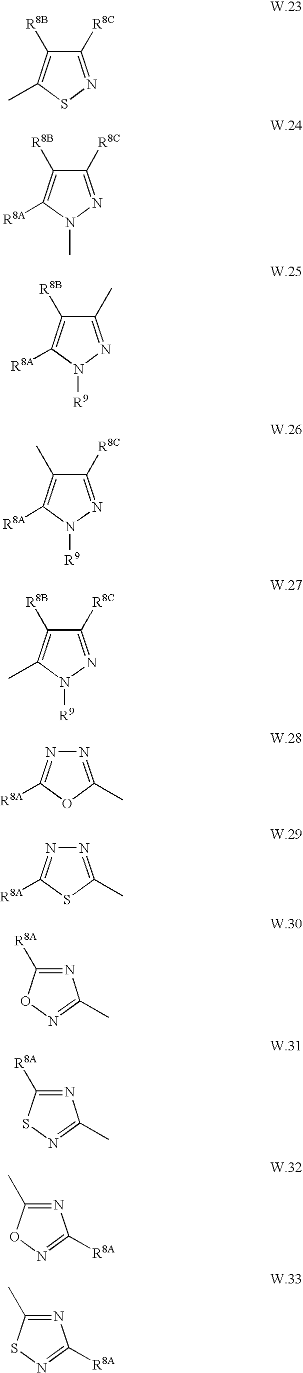

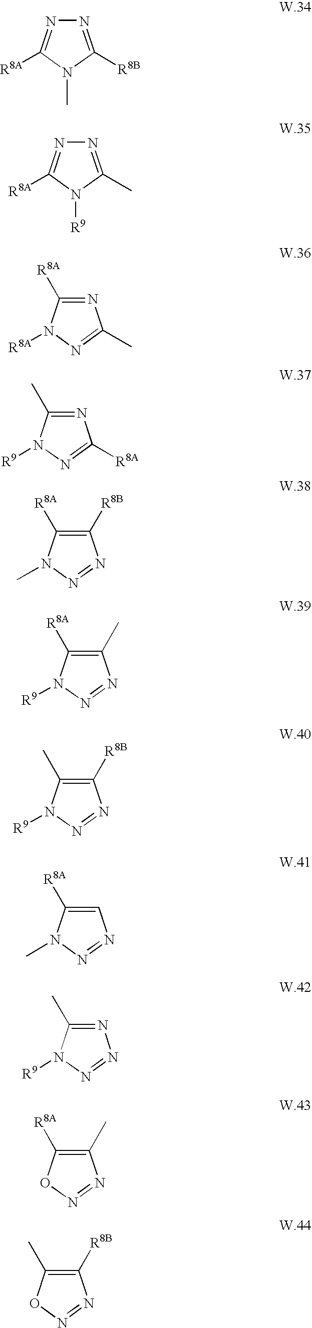

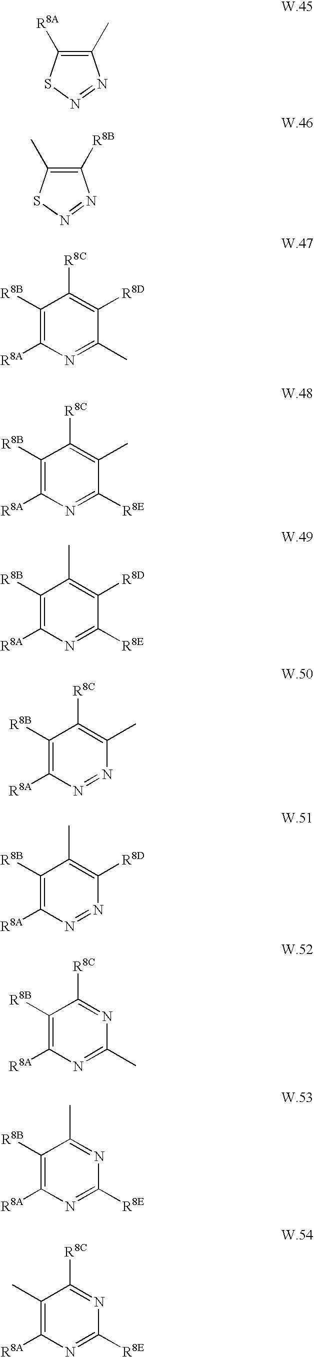

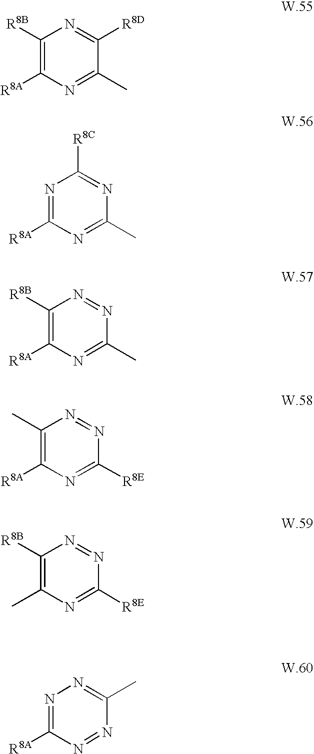

and wherein the free bond denotes the position of attachment in formula I.

More preferred are 1-(Azolin-2-yl)-amino-1-phenyl-2-hetaryl-ethane compounds, wherein W is selected from W.1, W.2, W.3, W.4, W.5, W.6, W.8, W.9, W.10, W.11, W.12, W.13, W.14, W.15, W.17, W.18, W.19, W.20, W.21, W.22, W.23, W.25, W.26, W.27, W.28, W.29, W.30, W.31, W.32, W.33, W.47, W.48, W.49, W.52, W.53 and W.54.

Most preferred are 1-(Azolin-2-yl)-amino-1-phenyl-2-hetaryl-ethane compounds, wherein W is selected from W.1, W.3, W.25, W.26, W.27, W.47, W.48, W.49, W.52, W.53 and W.54.

Preferred are also compounds of formula I, wherein W is selected from the following table H of monosubstituted heteroarylradicals (W-R.1-W.R.512):

TABLE H

| Radical | W | R^{8A} | R^{8B} | R^{8C} | R^{8D} | R^{8E} | R⁹ |
|---|---|---|---|---|---|---|---|
| W-R.1 | W.1 | H | H | H | / | / | / |
| W-R.2 | W.1 | CH₃ | H | H | / | / | / |
| W-R.3 | W.1 | H | CH₃ | H | / | / | / |
| W-R.4 | W.1 | H | H | CH₃ | / | / | / |
| W-R.5 | W.1 | CF₃ | H | H | / | / | / |
| W-R.6 | W.1 | H | CF₃ | H | / | / | / |
| W-R.7 | W.1 | H | H | CF₃ | / | / | / |
| W-R.8 | W.1 | Cl | H | H | / | / | / |
| W-R.9 | W.1 | H | Cl | H | / | / | / |
| W-R.10 | W.1 | H | H | Cl | / | / | / |
| W-R.11 | W.2 | H | H | / | H | / | / |
| W-R.12 | W.2 | CH₃ | H | / | H | / | / |
| W-R.13 | W.2 | H | CH₃ | / | H | / | / |
| W-R.14 | W.2 | H | H | / | CH₃ | / | / |
| W-R.15 | W.2 | CF₃ | H | / | H | / | / |
| W-R.16 | W.2 | H | CF₃ | / | H | / | / |
| W-R.17 | W.2 | H | H | / | CF₃ | / | / |
| W-R.18 | W.2 | Cl | H | / | H | / | / |
| W-R.19 | W.2 | H | Cl | / | H | / | / |
| W-R.20 | W.2 | H | H | / | Cl | / | / |
| W-R.21 | W.3 | H | H | H | / | / | / |
| W-R.22 | W.3 | CH₃ | H | H | / | / | / |
| W-R.23 | W.3 | H | CH₃ | H | / | / | / |
| W-R.24 | W.3 | H | H | CH₃ | / | / | / |
| W-R.25 | W.3 | CF₃ | H | H | / | / | / |
| W-R.26 | W.3 | H | CF₃ | H | / | / | / |
| W-R.27 | W.3 | H | H | CF₃ | / | / | / |
| W-R.28 | W.3 | Cl | H | H | / | / | / |
| W-R.29 | W.3 | H | Cl | H | / | / | / |
| W-R.30 | W.3 | H | H | Cl | / | / | / |
| W-R.31 | W.4 | H | H | / | H | / | / |
| W-R.32 | W.4 | CH₃ | H | / | H | / | / |
| W-R.33 | W.4 | H | CH₃ | / | H | / | / |
| W-R.34 | W.4 | H | H | / | CH₃ | / | / |
| W-R.35 | W.4 | CF₃ | H | / | H | / | / |
| W-R.36 | W.4 | H | CF₃ | / | H | / | / |
| W-R.37 | W.4 | H | H | / | CF₃ | / | / |
| W-R.38 | W.4 | Cl | H | / | H | / | / |
| W-R.39 | W.4 | H | Cl | / | H | / | / |
| W-R.40 | W.4 | H | H | / | Cl | / | / |
| W-R.41 | W.5 | H | H | H | / | / | H |
| W-R.42 | W.5 | CH₃ | H | H | / | / | H |
| W-R.43 | W.5 | H | CH₃ | H | / | / | H |
| W-R.44 | W.5 | H | H | CH₃ | / | / | H |
| W-R.45 | W.5 | CF₃ | H | H | / | / | H |
| W-R.46 | W.5 | H | CF₃ | H | / | / | H |
| W-R.47 | W.5 | H | H | CF₃ | / | / | H |
| W-R.48 | W.5 | Cl | H | H | / | / | H |
| W-R.49 | W.5 | H | Cl | H | / | / | H |
| W-R.50 | W.5 | H | H | Cl | / | / | H |
| W-R.51 | W.5 | H | H | H | / | / | CH₃ |
| W-R.52 | W.5 | CH₃ | H | H | / | / | CH₃ |
| W-R.53 | W.5 | H | CH₃ | H | / | / | CH₃ |
| W-R.54 | W.5 | H | H | CH₃ | / | / | CH₃ |
| W-R.55 | W.5 | CF₃ | H | H | / | / | CH₃ |
| W-R.56 | W.5 | H | CF₃ | H | / | / | CH₃ |
| W-R.57 | W.5 | H | H | CF₃ | / | / | CH₃ |
| W-R.58 | W.5 | Cl | H | H | / | / | CH₃ |
| W-R.59 | W.5 | H | Cl | H | / | / | CH₃ |
| W-R.60 | W.5 | H | H | Cl | / | / | CH₃ |
| W-R.61 | W.6 | H | H | / | H | / | H |
| W-R.62 | W.6 | CH₃ | H | / | H | / | H |
| W-R.63 | W.6 | H | CH₃ | / | H | / | H |
| W-R.64 | W.6 | H | H | / | CH₃ | / | H |
| W-R.65 | W.6 | CF₃ | H | / | H | / | H |
| W-R.66 | W.6 | H | CF₃ | / | H | / | H |
| W-R.67 | W.6 | H | H | / | CF₃ | / | H |
| W-R.68 | W.6 | Cl | H | / | H | / | H |
| W-R.69 | W.6 | H | Cl | / | H | / | H |
| W-R.70 | W.6 | H | H | / | Cl | / | H |
| W-R.71 | W.6 | H | H | / | H | / | CH₃ |
| W-R.72 | W.6 | CH₃ | H | / | H | / | CH₃ |
| W-R.73 | W.6 | H | CH₃ | / | H | / | CH₃ |
| W-R.74 | W.6 | H | H | / | CH₃ | / | CH₃ |
| W-R.75 | W.6 | CF₃ | H | / | H | / | CH₃ |
| W-R.76 | W.6 | H | CF₃ | / | H | / | CH₃ |
| W-R.77 | W.6 | H | H | / | CF₃ | / | CH₃ |
| W-R.78 | W.6 | Cl | H | / | H | / | CH₃ |
| W-R.79 | W.6 | H | Cl | / | H | / | CH₃ |
| W-R.80 | W.6 | H | H | / | Cl | / | CH₃ |
| W-R.81 | W.7 | H | H | H | H | / | / |
| W-R.82 | W.7 | CH₃ | H | H | H | / | / |
| W-R.83 | W.7 | H | CH₃ | H | H | / | / |
| W-R.84 | W.7 | H | H | CH₃ | H | / | / |
| W-R.85 | W.7 | H | H | H | CH₃ | / | / |
| W-R.86 | W.7 | CF₃ | H | H | H | / | / |
| W-R.87 | W.7 | H | CF₃ | H | H | / | / |
| W-R.88 | W.7 | H | H | CF₃ | H | / | / |
| W-R.89 | W.7 | H | H | H | CF₃ | / | / |
| W-R.90 | W.7 | Cl | H | H | H | / | / |
| W-R.91 | W.7 | H | Cl | H | H | / | / |
| W-R.92 | W.7 | H | H | Cl | H | / | / |
| W-R.93 | W.7 | H | H | H | Cl | / | / |
| W-R.94 | W.8 | H | H | / | / | / | / |
| W-R.95 | W.8 | H | CH₃ | / | / | / | / |
| W-R.96 | W.8 | CH₃ | H | / | / | / | / |
| W-R.97 | W.8 | H | CF₃ | / | / | / | / |
| W-R.98 | W.8 | CF₃ | H | / | / | / | / |
| W-R.99 | W.8 | H | Cl | / | / | / | / |
| W-R.100 | W.8 | Cl | H | / | / | / | / |
| W-R.101 | W.9 | H | / | / | H | / | / |
| W-R.102 | W.9 | CH₃ | / | / | H | / | / |
| W-R.103 | W.9 | H | / | / | CH₃ | / | / |
| W-R.104 | W.9 | CF₃ | / | / | H | / | / |
| W-R.105 | W.9 | H | / | / | CF₃ | / | / |
| W-R.106 | W.9 | Cl | / | / | H | / | / |
| W-R.107 | W.9 | H | / | / | Cl | / | / |
| W-R.108 | W.10 | / | H | / | H | / | / |
| W-R.109 | W.10 | / | H | / | CH₃ | / | / |
| W-R.110 | W.10 | / | CH₃ | / | H | / | / |
| W-R.111 | W.10 | / | H | / | CF₃ | / | / |
| W-R.112 | W.10 | / | CF₃ | / | H | / | / |
| W-R.113 | W.10 | / | H | / | Cl | / | / |
| W-R.114 | W.10 | / | Cl | / | H | / | / |
| W-R.115 | W.11 | H | H | / | / | / | / |
| W-R.116 | W.11 | H | CH₃ | / | / | / | / |
| W-R.117 | W.11 | CH₃ | H | / | / | / | / |
| W-R.118 | W.11 | H | CF₃ | / | / | / | / |
| W-R.119 | W.11 | CF₃ | H | / | / | / | / |
| W-R.120 | W.11 | H | Cl | / | / | / | / |
| W-R.121 | W.11 | Cl | H | / | / | / | / |
| W-R.122 | W.12 | H | / | / | H | / | / |
| W-R.123 | W.12 | CH₃ | / | / | H | / | / |
| W-R.124 | W.12 | H | / | / | CH₃ | / | / |
| W-R.125 | W.12 | CF₃ | / | / | H | / | / |
| W-R.126 | W.12 | H | / | / | CF₃ | / | / |
| W-R.127 | W.12 | Cl | / | / | H | / | / |
| W-R.128 | W.12 | H | / | / | Cl | / | / |
| W-R.129 | W.13 | / | H | / | H | / | / |
| W-R.130 | W.13 | / | H | / | CH₃ | / | / |
| W-R.131 | W.13 | / | CH₃ | / | H | / | / |
| W-R.132 | W.13 | / | H | / | CF₃ | / | / |
| W-R.133 | W.13 | / | CF₃ | / | H | / | / |
| W-R.134 | W.13 | / | H | / | Cl | / | / |
| W-R.135 | W.13 | / | Cl | / | H | / | / |
| W-R.136 | W.14 | H | H | / | / | / | H |
| W-R.137 | W.14 | H | CH₃ | / | / | / | H |
| W-R.138 | W.14 | CH₃ | H | / | / | / | H |
| W-R.139 | W.14 | H | CF₃ | / | / | / | H |
| W-R.140 | W.14 | CF₃ | H | / | / | / | H |
| W-R.141 | W.14 | H | Cl | / | / | / | H |
| W-R.142 | W.14 | Cl | H | / | / | / | H |
| W-R.143 | W.14 | H | H | / | / | / | CH₃ |
| W-R.144 | W.14 | H | CH₃ | / | / | / | CH₃ |
| W-R.145 | W.14 | CH₃ | H | / | / | / | CH₃ |
| W-R.146 | W.14 | H | CF₃ | / | / | / | CH₃ |
| W-R.147 | W.14 | CF₃ | H | / | / | / | CH₃ |
| W-R.148 | W.14 | H | Cl | / | / | / | CH₃ |
| W-R.149 | W.14 | Cl | H | / | / | / | CH₃ |
| W-R.150 | W.15 | H | / | / | H | / | H |
| W-R.151 | W.15 | CH₃ | / | / | H | / | H |
| W-R.152 | W.15 | H | / | / | CH₃ | / | H |
| W-R.153 | W.15 | CF₃ | / | / | H | / | H |
| W-R.154 | W.15 | H | / | / | CF₃ | / | H |
| W-R.155 | W.15 | Cl | / | / | H | / | H |
| W-R.156 | W.15 | H | / | / | Cl | / | H |
| W-R.157 | W.15 | H | / | / | H | / | CH₃ |
| W-R.158 | W.15 | CH₃ | / | / | H | / | CH₃ |
| W-R.159 | W.15 | H | / | / | CH₃ | / | CH₃ |
| W-R.160 | W.15 | CF₃ | / | / | H | / | CH₃ |
| W-R.161 | W.15 | H | / | / | CF₃ | / | CH₃ |
| W-R.162 | W.15 | Cl | / | / | H | / | CH₃ |
| W-R.163 | W.15 | H | / | / | Cl | / | CH₃ |
| W-R.164 | W.16 | / | H | / | H | / | H |
| W-R.165 | W.16 | / | H | / | CH₃ | / | H |
| W-R.166 | W.16 | / | CH₃ | / | H | / | H |
| W-R.167 | W.16 | / | H | / | CF₃ | / | H |
| W-R.168 | W.16 | / | CF₃ | / | H | / | H |
| W-R.169 | W.16 | / | H | / | Cl | / | H |
| W-R.170 | W.16 | / | Cl | / | H | / | H |
| W-R.171 | W.16 | / | H | / | H | / | CH₃ |
| W-R.172 | W.16 | / | H | / | CH₃ | / | CH₃ |
| W-R.173 | W.16 | / | CH₃ | / | H | / | CH₃ |
| W-R.174 | W.16 | / | H | / | CF₃ | / | CH₃ |
| W-R.175 | W.16 | / | CF₃ | / | H | / | CH₃ |
| W-R.176 | W.16 | / | H | / | Cl | / | CH₃ |
| W-R.177 | W.16 | / | Cl | / | H | / | CH₃ |
| W-R.178 | W.17 | H | H | / | H | / | / |
| W-R.179 | W.17 | CH₃ | H | / | H | / | / |
| W-R.180 | W.17 | H | CH₃ | / | H | / | / |
| W-R.181 | W.17 | H | H | / | CH₃ | / | / |
| W-R.182 | W.17 | CF₃ | H | / | H | / | / |
| W-R.183 | W.17 | H | CF₃ | / | H | / | / |
| W-R.184 | W.17 | H | H | / | CF₃ | / | / |
| W-R.185 | W.17 | Cl | H | / | H | / | / |
| W-R.186 | W.17 | H | Cl | / | H | / | / |
| W-R.187 | W.17 | H | H | / | Cl | / | / |
| W-R.188 | W.18 | H | H | / | / | / | / |
| W-R.189 | W.18 | H | CH₃ | / | / | / | / |
| W-R.190 | W.18 | CH₃ | H | / | / | / | / |
| W-R.191 | W.18 | H | CF₃ | / | / | / | / |
| W-R.192 | W.18 | CF₃ | H | / | / | / | / |
| W-R.193 | W.18 | H | Cl | / | / | / | / |
| W-R.194 | W.18 | Cl | H | / | / | / | / |
| W-R.195 | W.19 | H | / | H | / | / | / |
| W-R.196 | W.19 | CH₃ | / | H | / | / | / |
| W-R.197 | W.19 | H | / | CH₃ | / | / | / |
| W-R.198 | W.19 | CF₃ | / | H | / | / | / |
| W-R.199 | W.19 | H | / | CF₃ | / | / | / |
| W-R.200 | W.19 | Cl | / | H | / | / | / |
| W-R.201 | W.19 | H | / | Cl | / | / | / |
| W-R.202 | W.20 | / | H | H | / | / | / |
| W-R.203 | W.20 | / | CH₃ | H | / | / | / |
| W-R.204 | W.20 | / | H | CH₃ | / | / | / |
| W-R.205 | W.20 | / | CF₃ | H | / | / | / |
| W-R.206 | W.20 | / | H | CF₃ | / | / | / |
| W-R.207 | W.20 | / | Cl | H | / | / | / |
| W-R.208 | W.20 | / | H | Cl | / | / | / |
| W-R.209 | W.21 | H | H | / | / | / | / |
| W-R.210 | W.21 | H | CH₃ | / | / | / | / |
| W-R.211 | W.21 | CH₃ | H | / | / | / | / |
| W-R.212 | W.21 | H | CF₃ | / | / | / | / |
| W-R.213 | W.21 | CF₃ | H | / | / | / | / |
| W-R.214 | W.1 | H | Cl | / | / | / | / |
| W-R.215 | W.21 | Cl | H | / | / | / | / |
| W-R.216 | W.22 | H | / | H | / | / | / |
| W-R.217 | W.22 | CH₃ | / | H | / | / | / |
| W-R.218 | W.22 | H | / | CH₃ | / | / | / |
| W-R.219 | W.22 | CF₃ | / | H | / | / | / |
| W-R.220 | W.22 | H | / | CF₃ | / | / | / |
| W-R.221 | W.22 | Cl | / | H | / | / | / |
| W-R.222 | W.22 | H | / | Cl | / | / | / |
| W-R.223 | W.23 | / | H | H | / | / | / |
| W-R.224 | W.23 | / | CH₃ | H | / | / | / |
| W-R.225 | W.23 | / | H | CH₃ | / | / | / |
| W-R.226 | W.23 | / | CF₃ | H | / | / | / |
| W-R.227 | W.23 | / | H | CF₃ | / | / | / |
| W-R.228 | W.23 | / | Cl | H | / | / | / |
| W-R.229 | W.23 | / | H | Cl | / | / | / |
| W-R.230 | W.24 | H | H | H | / | / | / |
| W-R.231 | W.24 | CH₃ | H | H | / | / | / |
| W-R.232 | W.24 | H | CH₃ | H | / | / | / |
| W-R.233 | W.24 | H | H | CH₃ | / | / | / |
| W-R.234 | W.24 | CF₃ | H | H | / | / | / |
| W-R.235 | W.24 | H | CF₃ | H | / | / | / |
| W-R.236 | W.24 | H | H | CF₃ | / | / | / |
| W-R.237 | W.24 | Cl | H | H | / | / | / |
| W-R.238 | W.24 | H | Cl | H | / | / | / |
| W-R.239 | W.24 | H | H | Cl | / | / | / |
| W-R.240 | W.25 | H | H | / | / | / | H |
| W-R.241 | W.25 | H | CH₃ | / | / | / | H |
| W-R.242 | W.25 | CH₃ | H | / | / | / | H |
| W-R.243 | W.25 | H | CF₃ | / | / | / | H |
| W-R.244 | W.25 | CF₃ | H | / | / | / | H |
| W-R.245 | W.25 | H | Cl | / | / | / | H |
| W-R.246 | W.25 | Cl | H | / | / | / | H |
| W-R.247 | W.25 | H | H | / | / | / | CH₃ |
| W-R.248 | W.25 | H | CH₃ | / | / | / | CH₃ |
| W-R.249 | W.25 | CH₃ | H | / | / | / | CH₃ |
| W-R.250 | W.25 | H | CF₃ | / | / | / | CH₃ |
| W-R.251 | W.25 | CF₃ | H | / | / | / | CH₃ |
| W-R.252 | W.25 | H | Cl | / | / | / | CH₃ |
| W-R.253 | W.25 | Cl | H | / | / | / | CH₃ |
| W-R.254 | W.26 | H | / | H | / | / | H |
| W-R.255 | W.26 | CH₃ | / | H | / | / | H |
| W-R.256 | W.26 | H | / | CH₃ | / | / | H |
| W-R.257 | W.26 | CF₃ | / | H | / | / | H |
| W-R.258 | W.26 | H | / | CF₃ | / | / | H |
| W-R.259 | W.26 | Cl | / | H | / | / | H |
| W-R.260 | W.26 | H | / | Cl | / | / | H |
| W-R.261 | W.26 | H | / | H | / | / | CH₃ |
| W-R.262 | W.26 | CH₃ | / | H | / | / | CH₃ |
| W-R.263 | W.26 | H | / | CH₃ | / | / | CH₃ |
| W-R.264 | W.26 | CF₃ | / | H | / | / | CH₃ |
| W-R.265 | W.26 | H | / | CF₃ | / | / | CH₃ |
| W-R.266 | W.26 | Cl | / | H | / | / | CH₃ |
| W-R.267 | W.26 | H | / | Cl | / | / | CH₃ |
| W-R.268 | W.27 | / | H | H | / | / | H |
| W-R.269 | W.27 | / | CH₃ | H | / | / | H |
| W-R.270 | W.27 | / | H | CH₃ | / | / | H |
| W-R.271 | W.27 | / | CF₃ | H | / | / | H |
| W-R.272 | W.27 | / | H | CF₃ | / | / | H |
| W-R.273 | W.27 | / | Cl | H | / | / | H |
| W-R.274 | W.27 | / | H | Cl | / | / | H |
| W-R.275 | W.27 | / | H | H | / | / | CH₃ |
| W-R.276 | W.27 | / | CH₃ | H | / | / | CH₃ |
| W-R.277 | W.27 | / | H | CH₃ | / | / | CH₃ |
| W-R.278 | W.27 | / | CF₃ | H | / | / | CH₃ |
| W-R.279 | W.27 | / | H | CF₃ | / | / | CH₃ |
| W-R.280 | W.27 | / | Cl | H | / | / | CH₃ |
| W-R.281 | W.27 | / | H | Cl | / | / | CH₃ |
| W-R.282 | W.28 | H | / | / | / | / | / |
| W-R.283 | W.28 | CH₃ | / | / | / | / | / |
| W-R.284 | W.28 | CF₃ | / | / | / | / | / |
| W-R.285 | W.28 | Cl | / | / | / | / | / |
| W-R.286 | W.29 | H | / | / | / | / | / |
| W-R.287 | W.29 | CH₃ | / | / | / | / | / |
| W-R.288 | W.29 | CF₃ | / | / | / | / | / |
| W-R.289 | W.29 | Cl | / | / | / | / | / |
| W-R.290 | W.30 | H | / | / | / | / | / |
| W-R.291 | W.30 | CH₃ | / | / | / | / | / |
| W-R.292 | W.30 | CF₃ | / | / | / | / | / |
| W-R.293 | W.30 | Cl | / | / | / | / | / |
| W-R.294 | W.31 | H | / | / | / | / | / |
| W-R.295 | W.31 | CH₃ | / | / | / | / | / |
| W-R.296 | W.31 | CF₃ | / | / | / | / | / |
| W-R.297 | W.31 | Cl | / | / | / | / | / |
| W-R.298 | W.32 | H | / | / | / | / | / |
| W-R.299 | W.32 | CH₃ | / | / | / | / | / |
| W-R.300 | W.32 | CF₃ | / | / | / | / | / |
| W-R.301 | W.32 | Cl | / | / | / | / | / |
| W-R.302 | W.33 | H | / | / | / | / | / |
| W-R.303 | W.33 | CH₃ | / | / | / | / | / |
| W-R.304 | W.33 | CF₃ | / | / | / | / | / |
| W-R.305 | W.33 | Cl | / | / | / | / | / |
| W-R.306 | W.34 | H | H | / | / | / | / |
| W-R.307 | W.34 | H | CH₃ | / | / | / | / |
| W-R.308 | W.34 | CH₃ | H | / | / | / | / |
| W-R.309 | W.34 | H | CF₃ | / | / | / | / |
| W-R.310 | W.34 | CF₃ | H | / | / | / | / |
| W-R.311 | W.34 | H | Cl | / | / | / | / |
| W-R.312 | W.34 | Cl | H | / | / | / | / |
| W-R.313 | W.35 | H | / | / | / | / | H |
| W-R.314 | W.35 | CH₃ | / | / | / | / | H |
| W-R.315 | W.35 | CF₃ | / | / | / | / | H |
| W-R.316 | W.35 | Cl | / | / | / | / | H |
| W-R.317 | W.35 | H | / | / | / | / | CH₃ |
| W-R.318 | W.35 | CH₃ | / | / | / | / | CH₃ |
| W-R.319 | W.35 | CF₃ | / | / | / | / | CH₃ |
| W-R.320 | W.35 | Cl | / | / | / | / | CH₃ |
| W-R.321 | W.36 | H | / | / | / | / | H |
| W-R.322 | W.36 | CH₃ | / | / | / | / | H |
| W-R.323 | W.36 | CF₃ | / | / | / | / | H |
| W-R.324 | W.36 | Cl | / | / | / | / | H |
| W-R.325 | W.36 | H | / | / | / | / | CH₃ |
| W-R.326 | W.36 | CH₃ | / | / | / | / | CH₃ |
| W-R.327 | W.36 | CF₃ | / | / | / | / | CH₃ |
| W-R.328 | W.36 | Cl | / | / | / | / | CH₃ |
| W-R.329 | W.37 | H | / | / | / | / | H |
| W-R.330 | W.37 | CH₃ | / | / | / | / | H |
| W-R.331 | W.37 | CF₃ | / | / | / | / | H |
| W-R.332 | W.37 | Cl | / | / | / | / | H |
| W-R.333 | W.37 | H | / | / | / | / | CH₃ |
| W-R.334 | W.37 | CH₃ | / | / | / | / | CH₃ |
| W-R.335 | W.37 | CF₃ | / | / | / | / | CH₃ |
| W-R.336 | W.37 | Cl | / | / | / | / | CH₃ |
| W-R.337 | W.38 | H | H | / | / | / | / |
| W-R.338 | W.38 | H | CH₃ | / | / | / | / |
| W-R.339 | W.38 | CH₃ | H | / | / | / | / |
| W-R.340 | W.38 | H | CF₃ | / | / | / | / |
| W-R.341 | W.38 | CF₃ | H | / | / | / | / |
| W-R.342 | W.38 | H | Cl | / | / | / | / |
| W-R.343 | W.38 | Cl | H | / | / | / | / |
| W-R.344 | W.39 | H | / | / | / | / | H |
| W-R.345 | W.39 | CH₃ | / | / | / | / | H |
| W-R.346 | W.39 | CF₃ | / | / | / | / | H |
| W-R.347 | W.39 | Cl | / | / | / | / | H |
| W-R.348 | W.39 | H | / | / | / | / | CH₃ |
| W-R.349 | W.39 | CH₃ | / | / | / | / | CH₃ |
| W-R.350 | W.39 | CF₃ | / | / | / | / | CH₃ |
| W-R.351 | W.39 | Cl | / | / | / | / | CH₃ |
| W-R.352 | W.40 | / | H | / | / | / | H |
| W-R.353 | W.40 | / | CH₃ | / | / | / | H |
| W-R.354 | W.40 | / | CF₃ | / | / | / | H |
| W-R.355 | W.40 | / | Cl | / | / | / | H |
| W-R.356 | W.40 | / | H | / | / | / | CH₃ |
| W-R.357 | W.40 | / | CH₃ | / | / | / | CH₃ |
| W-R.358 | W.40 | / | CF₃ | / | / | / | CH₃ |
| W-R.359 | W.40 | / | Cl | / | / | / | CH₃ |
| W-R.360 | W.41 | H | / | / | / | / | / |
| W-R.361 | W.41 | CH₃ | / | / | / | / | / |
| W-R.362 | W.41 | CF₃ | / | / | / | / | / |
| W-R.363 | W.41 | Cl | / | / | / | / | / |
| W-R.364 | W.42 | / | / | / | / | / | H |
| W-R.365 | W.42 | / | / | / | / | / | CH₃ |
| W-R.366 | W.43 | H | / | / | / | / | / |
| W-R.367 | W.43 | CH₃ | / | / | / | / | / |
| W-R.368 | W.43 | CF₃ | / | / | / | / | / |
| W-R.369 | W.43 | Cl | / | / | / | / | / |
| W-R.370 | W.44 | / | H | / | / | / | / |
| W-R.371 | W.44 | / | CH₃ | / | / | / | / |
| W-R.372 | W.44 | / | CF₃ | / | / | / | / |
| W-R.373 | W.44 | / | Cl | / | / | / | / |
| W-R.374 | W.45 | H | / | / | / | / | / |
| W-R.375 | W.45 | CH₃ | / | / | / | / | / |
| W-R.376 | W.45 | CF₃ | / | / | / | / | / |
| W-R.377 | W.45 | Cl | / | / | / | / | / |
| W-R.378 | W.46 | / | H | / | / | / | / |
| W-R.379 | W.46 | / | CH₃ | / | / | / | / |
| W-R.380 | W.46 | / | CF₃ | / | / | / | / |
| W-R.381 | W.46 | / | Cl | / | / | / | / |
| W-R.382 | W.47 | H | H | H | H | / | / |
| W-R.383 | W.47 | CH₃ | H | H | H | / | / |
| W-R.384 | W.47 | H | CH₃ | H | H | / | / |
| W-R.385 | W.47 | H | H | CH₃ | H | / | / |
| W-R.386 | W.47 | H | H | H | CH₃ | / | / |
| W-R.387 | W.47 | CF₃ | H | H | H | / | / |
| W-R.388 | W.47 | H | CF₃ | H | H | / | / |
| W-R.389 | W.47 | H | H | CF₃ | H | / | / |
| W-R.390 | W.47 | H | H | H | CF₃ | / | / |
| W-R.391 | W.47 | Cl | H | H | H | / | / |
| W-R.392 | W.47 | H | Cl | H | H | / | / |
| W-R.393 | W.47 | H | H | Cl | H | / | / |
| W-R.394 | W.47 | H | H | H | Cl | / | / |
| W-R.395 | W.48 | H | H | H | / | H | / |
| W-R.396 | W.48 | CH₃ | H | H | / | H | / |
| W-R.397 | W.48 | H | CH₃ | H | / | H | / |
| W-R.398 | W.48 | H | H | CH₃ | / | H | / |
| W-R.399 | W.48 | H | H | H | / | CH₃ | / |
| W-R.400 | W.48 | CF₃ | H | H | / | H | / |
| W-R.401 | W.48 | H | CF₃ | H | / | H | / |
| W-R.402 | W.48 | H | H | CF₃ | / | H | / |
| W-R.403 | W.48 | H | H | H | / | CF₃ | / |
| W-R.404 | W.48 | Cl | H | H | / | H | / |
| W-R.405 | W.48 | H | Cl | H | / | H | / |
| W-R.406 | W.48 | H | H | Cl | / | H | / |
| W-R.407 | W.48 | H | H | H | / | Cl | / |
| W-R.408 | W.49 | H | H | / | H | H | / |
| W-R.409 | W.49 | CH₃ | H | / | H | H | / |
| W-R.410 | W.49 | H | CH₃ | / | H | H | / |
| W-R.411 | W.49 | H | H | / | CH₃ | H | / |
| W-R.412 | W.49 | H | H | / | H | CH₃ | / |
| W-R.413 | W.49 | CF₃ | H | / | H | H | / |
| W-R.414 | W.49 | H | CF₃ | / | H | H | / |
| W-R.415 | W.49 | H | H | / | CF₃ | H | / |
| W-R.416 | W.49 | H | H | / | H | CF₃ | / |
| W-R.417 | W.49 | Cl | H | / | H | H | / |
| W-R.418 | W.49 | H | Cl | / | H | H | / |
| W-R.419 | W.49 | H | H | / | Cl | H | / |
| W-R.420 | W.49 | H | H | / | H | Cl | / |
| W-R.421 | W.50 | H | H | H | / | / | / |
| W-R.422 | W.50 | CH₃ | H | H | / | / | / |
| W-R.423 | W.50 | H | CH₃ | H | / | / | / |
| W-R.424 | W.50 | H | H | CH₃ | / | / | / |
| W-R.425 | W.50 | CF₃ | H | H | / | / | / |
| W-R.426 | W.50 | H | CF₃ | H | / | / | / |
| W-R.427 | W.50 | H | H | CF₃ | / | / | / |
| W-R.428 | W.50 | Cl | H | H | / | / | / |
| W-R.429 | W.50 | H | Cl | H | / | / | / |
| W-R.430 | W.50 | H | H | Cl | / | / | / |
| W-R.431 | W.51 | H | H | / | H | / | / |
| W-R.432 | W.51 | CH₃ | H | / | H | / | / |
| W-R.433 | W.51 | H | CH₃ | / | H | / | / |
| W-R.434 | W.51 | H | H | / | CH₃ | / | / |
| W-R.435 | W.51 | CF₃ | H | / | H | / | / |
| W-R.436 | W.51 | H | CF₃ | / | H | / | / |
| W-R.437 | W.51 | H | H | / | CF₃ | / | / |
| W-R.438 | W.51 | Cl | H | / | H | / | / |
| W-R.439 | W.51 | H | Cl | / | H | / | / |
| W-R.440 | W.51 | H | H | / | Cl | / | / |
| W-R.441 | W.52 | H | H | H | / | / | / |
| W-R.442 | W.52 | CH₃ | H | H | / | / | / |
| W-R.443 | W.52 | H | CH₃ | H | / | / | / |
| W-R.444 | W.52 | H | H | CH₃ | / | / | / |
| W-R.445 | W.52 | CF₃ | H | H | / | / | / |
| W-R.446 | W.52 | H | CF₃ | H | / | / | / |
| W-R.447 | W.52 | H | H | CF₃ | / | / | / |
| W-R.448 | W.52 | Cl | H | H | / | / | / |
| W-R.449 | W.52 | H | Cl | H | / | / | / |
| W-R.450 | W.52 | H | H | Cl | / | / | / |
| W-R.451 | W.53 | H | H | / | / | H | / |
| W-R.452 | W.53 | CH₃ | H | / | / | H | / |
| W-R.453 | W.53 | H | CH₃ | / | / | H | / |
| W-R.454 | W.53 | H | H | / | / | CH₃ | / |
| W-R.455 | W.53 | CF₃ | H | / | / | H | / |
| W-R.456 | W.53 | H | CF₃ | / | / | H | / |
| W-R.457 | W.53 | H | H | / | / | CF₃ | / |
| W-R.458 | W.53 | Cl | H | / | / | H | / |
| W-R.459 | W.53 | H | Cl | / | / | H | / |
| W-R.460 | W.53 | H | H | / | / | Cl | / |
| W-R.461 | W.54 | H | / | H | / | H | / |
| W-R.462 | W.54 | CH₃ | / | H | / | H | / |
| W-R.463 | W.54 | H | / | CH₃ | / | H | / |
| W-R.464 | W.54 | H | / | H | / | CH₃ | / |
| W-R.465 | W.54 | CF₃ | / | H | / | H | / |
| W-R.466 | W.54 | H | / | CF₃ | / | H | / |
| W-R.467 | W.54 | H | / | H | / | CF₃ | / |
| W-R.468 | W.54 | Cl | / | H | / | H | / |
| W-R.469 | W.54 | H | / | Cl | / | H | / |
| W-R.470 | W.54 | H | / | H | / | Cl | / |
| W-R.471 | W.55 | H | H | / | H | / | / |
| W-R.472 | W.55 | CH₃ | H | / | H | / | / |
| W-R.473 | W.55 | H | CH₃ | / | H | / | / |
| W-R.474 | W.55 | H | H | / | CH₃ | / | / |
| W-R.475 | W.55 | CF₃ | H | / | H | / | / |
| W-R.476 | W.55 | H | CF₃ | / | H | / | / |
| W-R.477 | W.55 | H | H | / | CF₃ | / | / |
| W-R.478 | W.55 | Cl | H | / | H | / | / |
| W-R.479 | W.55 | H | Cl | / | H | / | / |
| W-R.480 | W.55 | H | H | / | Cl | / | / |
| W-R.481 | W.56 | H | / | H | / | / | / |
| W-R.482 | W.56 | CH₃ | / | H | / | / | / |
| W-R.483 | W.56 | H | / | CH₃ | / | / | / |
| W-R.484 | W.56 | CF₃ | / | H | / | / | / |
| W-R.485 | W.56 | H | / | CF₃ | / | / | / |
| W-R.486 | W.56 | Cl | / | H | / | / | / |
| W-R.487 | W.56 | H | / | Cl | / | / | / |
| W-R.488 | W.57 | H | H | / | / | / | / |
| W-R.489 | W.57 | H | CH₃ | / | / | / | / |
| W-R.490 | W.57 | CH₃ | H | / | / | / | / |
| W-R.491 | W.57 | H | CF₃ | / | / | / | / |
| W-R.492 | W.57 | CF₃ | H | / | / | / | / |
| W-R.493 | W.57 | H | Cl | / | / | / | / |
| W-R.494 | W.57 | Cl | H | / | / | / | / |
| W-R.495 | W.58 | H | / | / | / | H | / |
| W-R.496 | W.58 | CH₃ | / | / | / | H | / |
| W-R.497 | W.58 | H | / | / | / | CH₃ | / |
| W-R.498 | W.58 | CF₃ | / | / | / | H | / |
| W-R.499 | W.58 | H | / | / | / | CF₃ | / |
| W-R.500 | W.58 | Cl | / | / | / | H | / |
| W-R.501 | W.58 | H | / | / | / | Cl | / |
| W-R.502 | W.59 | / | H | / | / | H | / |
| W-R.503 | W.59 | / | CH₃ | / | / | H | / |
| W-R.504 | W.59 | / | H | / | / | CH₃ | / |
| W-R.505 | W.59 | / | CF₃ | / | / | H | / |
| W-R.506 | W.59 | / | H | / | / | CF₃ | / |
| W-R.507 | W.59 | / | Cl | / | / | H | / |
| W-R.508 | W.59 | / | H | / | / | Cl | / |
| W-R.509 | W.60 | H | / | / | / | / | / |
| W-R.510 | W.60 | CH₃ | / | / | / | / | / |
| W-R.511 | W.60 | CF₃ | / | / | / | / | / |
| W-R.512 | W.60 | Cl | / | / | / | / | / |

Preferred 1-(Azolin-2-yl)-amino-1-phenyl-2-hetaryl-ethane compounds are described in the following tables:

### 1. Tables 1-1910:

Compounds of formula I, wherein the hetaryl radical derivative W corresponds to any W.1 radical defined as W-R.1 to W-R.10 in table H and the phenyl radical derivative B corresponds to any phenyl radical B.0 to B.190 in lists a), b) c) or d).

### 2. Tables 1911-3820:

Compounds of formula I, wherein the hetaryl radical derivative W corresponds to any W.2 radical defined as W-R.1 to W-R.20 in table H and the phenyl radical derivative B corresponds to any phenyl radical B.0 to B.190 in lists a), b) c) or d).

### 3. Tables 3821-5730:

Compounds of formula I, wherein the hetaryl radical derivative W corresponds to any W.3 radical defined as W-R.21 to W-R.30 in table H and the phenyl radical derivative B corresponds to any phenyl radical B.0 to B.190 in lists a), b) c) or d).

### 4. Tables 5731-7640:

Compounds of formula I, wherein the hetaryl radical derivative W corresponds to any W.4 radical defined as W-R.31 to W-R.40 in table H and the phenyl radical derivative B corresponds to any phenyl radical B.0 to B.190 in lists a), b) c) or d).

### 5. Tables 7641-11460:

Compounds of formula I, wherein the hetaryl radical derivative W corresponds to any W.5 radical defined as W-R.41 to W-R.60 in table H and the phenyl radical derivative B corresponds to any phenyl radical B.0 to B.190 in lists a), b) c) or d).

### 6. Tables 11461-15280:

Compounds of formula I, wherein the hetaryl radical derivative W corresponds to any W.6 radical defined as W-R.61 to W-R.80 in table H and the phenyl radical derivative B corresponds to any phenyl radical B.0 to B.190 in lists a), b) c) or d).

### 7. Tables 15281-17763:

Compounds of formula I, wherein the hetaryl radical derivative W corresponds to any W.7 radical defined as W-R.81 to W-R.93 in table H and the phenyl radical derivative B corresponds to any phenyl radical B.0 to B.190 in lists a), b) c) or d).

### 8. Tables 17764-19100:

Compounds of formula I, wherein the hetaryl radical derivative W corresponds to any W.8 radical defined as W-R.94 to W-R.100 in table H and the phenyl radical derivative B corresponds to any phenyl radical B.0 to B.190 in lists a), b) c) or d).

### 9. Tables 19101-20437:

Compounds of formula I, wherein the hetaryl radical derivative W corresponds to any W.9 radical defined as W-R.101 to W-R.107 in table H and the phenyl radical derivative B corresponds to any phenyl radical B.0 to B.190 in lists a), b) c) or d).

### 10. Tables 20438-21774:

Compounds of formula I, wherein the hetaryl radical derivative W corresponds to any W.10 radical defined as W-R.108 to W-R.114 in table H and the phenyl radical derivative B corresponds to any phenyl radical B.0 to B.190 in lists a), b) c) or d).

### 11. Tables 21775-23111:

Compounds of formula I, wherein the hetaryl radical derivative W corresponds to any W.11 radical defined as W-R.115 to W-R.121 in table H and the phenyl radical derivative B corresponds to any phenyl radical B.0 to B.190 in lists a), b) c) or d).

### 12. Tables 23112-24448:

Compounds of formula I, wherein the hetaryl radical derivative W corresponds to any W.12 radical defined as W-R.122 to W-R.128 in table H and the phenyl radical derivative B corresponds to any phenyl radical B.0 to B.190 in lists a), b) c) or d).

### 13. Tables 24449-25785:

Compounds of formula I, wherein the hetaryl radical derivative W corresponds to any W.13 radical defined as W-R.129 to W-R.135 in table H and the phenyl radical derivative B corresponds to any phenyl radical B.0 to B.190 in lists a), b) c) or d).

### 14. Tables 25786-28459:

Compounds of formula I, wherein the hetaryl radical derivative W corresponds to any W.14 radical defined as W-R.136 to W-R.149 in table H and the phenyl radical derivative B corresponds to any phenyl radical B.0 to B.190 in lists a), b) c) or d).

### 15. Tables 28460-31133:

Compounds of formula I, wherein the hetaryl radical derivative W corresponds to any W.15 radical defined as W-R.150 to W-R.163 in table H and the phenyl radical derivative B corresponds to any phenyl radical B.0 to B.190 in lists a), b) c) or d).

### 16. Tables 31134-33807:

Compounds of formula I, wherein the hetaryl radical derivative W corresponds to any W.16 radical defined as W-R.164 to W-R.177 in table H and the phenyl radical derivative B corresponds to any phenyl radical B.0 to B.190 in lists a), b) c) or d).

### 17. Tables 33808-35717:

Compounds of formula I, wherein the hetaryl radical derivative W corresponds to any W.17 radical defined as W-R.178 to W-R.187 in table H and the phenyl radical derivative B corresponds to any phenyl radical B.0 to B.190 in lists a), b) c) or d).

### 18. Tables 35718-37054:

Compounds of formula I, wherein the hetaryl radical derivative W corresponds to any W.18 radical defined as W-R.188 to W-R.194 in table H and the phenyl radical derivative B corresponds to any phenyl radical B.0 to B.190 in lists a), b) c) or d).

### 19. Tables 37055-38391:

Compounds of formula I, wherein the hetaryl radical derivative W corresponds to any W.19 radical defined as W-R.195 to W-R.201 in table H and the phenyl radical derivative B corresponds to any phenyl radical B.0 to B.190 in lists a), b) c) or d).

### 20. Tables 38392-39728:

Compounds of formula I, wherein the hetaryl radical derivative W corresponds to any W.20 radical defined as W-R.202 to W-R.208 in table H and the phenyl radical derivative B corresponds to any phenyl radical B.0 to B.190 in lists a), b) c) or d).

### 21. Tables 39729-41065:

Compounds of formula I, wherein the hetaryl radical derivative W corresponds to any W.21 radical defined as W-R.209 to W-R.215 in table H and the phenyl radical derivative B corresponds to any phenyl radical B.0 to B.190 in lists a), b) c) or d).

### 22. Tables 41066-42024:

Compounds of formula I, wherein the hetaryl radical derivative W corresponds to any W.22 radical defined as W-R.216 to W-R.222 in table H and the phenyl radical derivative B corresponds to any phenyl radical B.0 to B.190 in lists a), b) c) or d).

### 23. Tables 42403-43739:

Compounds of formula I, wherein the hetaryl radical derivative W corresponds to any W.23 radical defined as W-R.223 to W-R.229 in table H and the phenyl radical derivative B corresponds to any phenyl radical B.0 to B.190 in lists a), b) c) or d).

### 24. Tables 43740-45649:

Compounds of formula I, wherein the hetaryl radical derivative W corresponds to any W.24 radical defined as W-R.230 to W-R.239 in table H and the phenyl radical derivative B corresponds to any phenyl radical B.0 to B.190 in lists a), b) c) or d).

### 25. Tables 45650-48323:

Compounds of formula I, wherein the hetaryl radical derivative W corresponds to any W.25 radical defined as W-R.240 to W-R.253 in table H and the phenyl radical derivative B corresponds to any phenyl radical B.0 to B.190 in lists a), b) c) or d).

### 26. Tables 48324-50997:

Compounds of formula I, wherein the hetaryl radical derivative W corresponds to any W.26 radical defined as W-R.254 to W-R.267 in table H and the phenyl radical derivative B corresponds to any phenyl radical B.0 to B.190 in lists a), b) c) or d).

### 27. Tables 50998-53671:

Compounds of formula I, wherein the hetaryl radical derivative W corresponds to any W.27 radical defined as W-R.268 to W-R.281 in table H and the phenyl radical derivative B corresponds to any phenyl radical B.0 to B.190 in lists a), b) c) or d).

### 28. Tables 53672-54435:

Compounds of formula I, wherein the hetaryl radical derivative W corresponds to any W.28 radical defined as W-R.282 to W-R.285 in table H and the phenyl radical derivative B corresponds to any phenyl radical B.0 to B.190 in lists a), b) c) or d).

### 29. Tables 54436-55199:

Compounds of formula I, wherein the hetaryl radical derivative W corresponds to any W.29 radical defined as W-R.286 to W-R.289 in table H and the phenyl radical derivative B corresponds to any phenyl radical B.0 to B.190 in lists a), b) c) or d).

### 30. Tables 55200-55963:

Compounds of formula I, wherein the hetaryl radical derivative W corresponds to any W.30 radical defined as W-R.290 to W-R.293 in table H and the phenyl radical derivative B corresponds to any phenyl radical B.0 to B.190 in lists a), b) c) or d).

### 31. Tables 55964-56727:

Compounds of formula I, wherein the hetaryl radical derivative W corresponds to any W.31 radical defined as W-R.294 to W-R.297 in table H and the phenyl radical derivative B corresponds to any phenyl radical B.0 to B.190 in lists a), b) c) or d).

### 32. Tables 56728-57491:

Compounds of formula I, wherein the hetaryl radical derivative W corresponds to any W.32 radical defined as W-R.298 to W-R.301 in table H and the phenyl radical derivative B corresponds to any phenyl radical B.0 to B.190 in lists a), b) c) or d).

### 33. Tables 57492-58255:

Compounds of formula I, wherein the hetaryl radical derivative W corresponds to any W.33 radical defined as W-R.302 to W-R.305 in table H and the phenyl radical derivative B corresponds to any phenyl radical B.0 to B.190 in lists a), b) c) or d).

### 34. Tables 58256-59592:

Compounds of formula I, wherein the hetaryl radical derivative W corresponds to any W.34 radical defined as W-R.306 to W-R.312 in table H and the phenyl radical derivative B corresponds to any phenyl radical B.0 to B.190 in lists a), b) c) or d).

### 35. Tables 59593-61120:

Compounds of formula I, wherein the hetaryl radical derivative W corresponds to any W.35 radical defined as W-R.313 to W-R.320 in table H and the phenyl radical derivative B corresponds to any phenyl radical B.0 to B.190 in lists a), b) c) or d).

### 36. Tables 61121-62648:

Compounds of formula I, wherein the hetaryl radical derivative W corresponds to any W.36 radical defined as W-R.321 to W-R.328 in table H and the phenyl radical derivative B corresponds to any phenyl radical B.0 to B.190 in lists a), b) c) or d).

### 37. Tables 62649-64179:

Compounds of formula I, wherein the hetaryl radical derivative W corresponds to any W.37 radical defined as W-R.329 to W-R.336 in table H and the phenyl radical derivative B corresponds to any phenyl radical B.0 to B.190 in lists a), b) c) or d).

### 38. Tables 64177-65513:

Compounds of formula I, wherein the hetaryl radical derivative W corresponds to any W.38 radical defined as W-R.337 to W-R.343 in table H and the phenyl radical derivative B corresponds to any phenyl radical B.0 to B.190 in lists a), b) c) or d).

### 39. Tables 65514-67041:

Compounds of formula I, wherein the hetaryl radical derivative W corresponds to any W.39 radical defined as W-R.344 to W-R.351 in table H and the phenyl radical derivative B corresponds to any phenyl radical B.0 to B.190 in lists a), b) c) or d).

### 40. Tables 67042-68569:

Compounds of formula I, wherein the hetaryl radical derivative W corresponds to any W.41 radical defined as W-R.352 to W-R.359 in table H and the phenyl radical derivative B corresponds to any phenyl radical B.0 to B.190 in lists a), b) c) or d).

### 41. Tables 68570-69333:

Compounds of formula I, wherein the hetaryl radical derivative W corresponds to any W.41 radical defined as W-R.360 to W-R.363 in table H and the phenyl radical derivative B corresponds to any phenyl radical B.0 to B.190 in lists a), b) c) or d).

### 42. Tables 69334-69715:

Compounds of formula I, wherein the hetaryl radical derivative W corresponds to any W.42 radical defined as W-R.364 and W-R.365 in table H and the phenyl radical derivative B corresponds to any phenyl radical B.0 to B.190 in lists a), b) c) or d).

### 43. Tables 69716-70479:

Compounds of formula I, wherein the hetaryl radical derivative W corresponds to any W.43 radical defined as W-R.366 to W-R.369 in table H and the phenyl radical derivative B corresponds to any phenyl radical B.0 to B.190 in lists a), b) c) or d).

### 44. Tables 70480-71243:

Compounds of formula I, wherein the hetaryl radical derivative W corresponds to any W.44 radical defined as W-R.370 to W-R.373 in table H and the phenyl radical derivative B corresponds to any phenyl radical B.0 to B.190 in lists a), b) c) or d).

### 45. Tables 71243-72007:

Compounds of formula I, wherein the hetaryl radical derivative W corresponds to any W.45 radical defined as W-R.374 to W-R.377 in table H and the phenyl radical derivative B corresponds to any phenyl radical B.0 to B.190 in lists a), b) c) or d).

### 46. Tables 72008-72771:

Compounds of formula I, wherein the hetaryl radical derivative W corresponds to any W.46 radical defined as W-R.378 to W-R.381 in table H and the phenyl radical derivative B corresponds to any phenyl radical B.0 to B.190 in lists a), b) c) or d).

### 47. Tables 72772-75254:

Compounds of formula I, wherein the hetaryl radical derivative W corresponds to any W.47 radical defined as W-R.382 to W-R.394 in table H and the phenyl radical derivative B corresponds to any phenyl radical B.0 to B.190 in lists a), b) c) or d).

### 48. Tables 75255-77737:

Compounds of formula I, wherein the hetaryl radical derivative W corresponds to any W.48 radical defined as W-R.395 to W-R.407 in table H and the phenyl radical derivative B corresponds to any phenyl radical B.0 to B.190 in lists a), b) c) or d).

### 49. Tables 77738-80220:

Compounds of formula I, wherein the hetaryl radical derivative W corresponds to any W.49 radical defined as W-R.408 to W-R.420 in table H and the phenyl radical derivative B corresponds to any phenyl radical B.0 to B.190 in lists a), b) c) or d).

### 50. Tables 80221-82130:

Compounds of formula I, wherein the hetaryl radical derivative W corresponds to any W.50 radical defined as W-R.421 to W-R.430 in table H and the phenyl radical derivative B corresponds to any phenyl radical B.0 to B.190 in lists a), b) c) or d).

### 51. Tables 82131-84040:

Compounds of formula I, wherein the hetaryl radical derivative W corresponds to any W.51 radical defined as W-R.431 to W-R.440 in table H and the phenyl radical derivative B corresponds to any phenyl radical B.0 to B.190 in lists a), b) c) or d).

### 52. Tables 84041-85950:

Compounds of formula I, wherein the hetaryl radical derivative W corresponds to any W.52 radical defined as W-R.441 to W-R.450 in table H and the phenyl radical derivative B corresponds to any phenyl radical B.0 to B.190 in lists a), b) c) or d).

### 53. Tables 85951-87860:

Compounds of formula I, wherein the hetaryl radical derivative W corresponds to any W.53 radical defined as W-R.451 to W-R.460 in table H and the phenyl radical derivative B corresponds to any phenyl radical B.0 to B.190 in lists a), b) c) or d).

### 54. Tables 87861-89770:

Compounds of formula I, wherein the hetaryl radical derivative W corresponds to any W.54 radical defined as W-R.461 to W-R.470 in table H and the phenyl radical derivative B corresponds to any phenyl radical B.0 to B.190 in lists a), b) c) or d).

### 55. Tables 89771-91680:

Compounds of formula I, wherein the hetaryl radical derivative W corresponds to any W.55 radical defined as W-R.471 to W-R.480 in table H and the phenyl radical derivative B corresponds to any phenyl radical B.0 to B.190 in lists a), b) c) or d).

### 56. Tables 91681-93017:

Compounds of formula I, wherein the hetaryl radical derivative W corresponds to any W.56 radical defined as W-R.481 to W-R.487 in table H and the phenyl radical derivative B corresponds to any phenyl radical B.0 to B.190 in lists a), b) c) or d).

### 57. Tables 93018-94354:

Compounds of formula I, wherein the hetaryl radical derivative W corresponds to any W.57 radical defined as W-R.488 to W-R.494 in table H and the phenyl radical derivative B corresponds to any phenyl radical B.0 to B.190 in lists a), b) c) or d).

### 58. Tables 94355-95691:

Compounds of formula I, wherein the hetaryl radical derivative W corresponds to any W.58 radical defined as W-R.495 to W-R.501 in table H and the phenyl radical derivative B corresponds to any phenyl radical B.0 to B.190 in lists a), b) c) or d).

### 59. Tables 95692-97028:

Compounds of formula I, wherein the hetaryl radical derivative W corresponds to any W.59 radical defined as W-R.502 to W-R.508 in table H and the phenyl radical derivative B corresponds to any phenyl radical B.0 to B.190 in lists a), b) c) or d).

### 60. Tables 97029-97792:

Compounds of formula I, wherein the hetaryl radical derivative W corresponds to any W.60 radical defined as W-R.509 to W-R.512 in table H and the phenyl radical derivative B corresponds to any phenyl radical B.0 to B.190 in lists a), b) c) or d).

Especially preferred are compounds of formula I defined in the following table A, wherein R¹, R² and R³ are hydrogen;

A is A² and R^{4a}, R^{4b}, R^{4c} and R^{4d} are hydrogen and X is sulfur; and wherein R⁶ is hydrogen:

TABLE A

| Formula I | Phenyl radical B | Hetaryl radical W |
|---|---|---|
| formula I.1 | B.0 | W-R.1 |
| formula I.2 | B.1 | W-R.1 |
| formula I.3 | B.2 | W-R.1 |
| formula I.4 | B.3 | W-R.1 |
| formula I.5 | B.4 | W-R.1 |
| formula I.6 | B.5 | W-R.1 |
| formula I.7 | B.6 | W-R.1 |
| formula I.8 | B.7 | W-R.1 |
| formula I.9 | B.8 | W-R.1 |
| formula I.10 | B.9 | W-R.1 |
| formula I.11 | B.10 | W-R.1 |
| formula I.12 | B.11 | W-R.1 |
| formula I.13 | B.12 | W-R.1 |
| formula I.14 | B.13 | W-R.1 |
| formula I.15 | B.14 | W-R.1 |
| formula I.16 | B.15 | W-R.1 |
| formula I.17 | B.16 | W-R.1 |
| formula I.18 | B.17 | W-R.1 |
| formula I.19 | B.18 | W-R.1 |
| formula I.20 | B.19 | W-R.1 |
| formula I.21 | B.20 | W-R.1 |
| formula I.22 | B.21 | W-R.1 |
| formula I.23 | B.22 | W-R.1 |
| formula I.24 | B.23 | W-R.1 |
| formula I.25 | B.24 | W-R.1 |
| formula I.26 | B.25 | W-R.1 |
| formula I.27 | B.26 | W-R.1 |
| formula I.28 | B.27 | W-R.1 |
| formula I.29 | B.28 | W-R.1 |
| formula I.30 | B.29 | W-R.1 |
| formula I.31 | B.30 | W-R.1 |
| formula I.32 | B.31 | W-R.1 |
| formula I.33 | B.32 | W-R.1 |
| formula I.34 | B.33 | W-R.1 |
| formula I.35 | B.34 | W-R.1 |
| formula I.36 | B.35 | W-R.1 |
| formula I.37 | B.36 | W-R.1 |
| formula I.38 | B.37 | W-R.1 |
| formula I.39 | B.38 | W-R.1 |
| formula I.40 | B.39 | W-R.1 |
| formula I.41 | B.40 | W-R.1 |
| formula I.42 | B.41 | W-R.1 |
| formula I.43 | B.42 | W-R.1 |
| formula I.44 | B.43 | W-R.1 |
| formula I.45 | B.44 | W-R.1 |
| formula I.46 | B.45 | W-R.1 |
| formula I.47 | B.46 | W-R.1 |
| formula I.48 | B.47 | W-R.1 |
| formula I.49 | B.48 | W-R.1 |
| formula I.50 | B.49 | W-R.1 |
| formula I.51 | B.50 | W-R.1 |
| formula I.52 | B.51 | W-R.1 |
| formula I.53 | B.52 | W-R.1 |
| formula I.54 | B.53 | W-R.1 |
| formula I.55 | B.54 | W-R.1 |
| formula I.56 | B.55 | W-R.1 |
| formula I.57 | B.56 | W-R.1 |
| formula I.58 | B.57 | W-R.1 |
| formula I.59 | B.58 | W-R.1 |
| formula I.60 | B.59 | W-R.1 |
| formula I.61 | B.60 | W-R.1 |
| formula I.62 | B.61 | W-R.1 |
| formula I.63 | B.62 | W-R.1 |
| formula I.64 | B.63 | W-R.1 |
| formula I.65 | B.64 | W-R.1 |
| formula I.66 | B.65 | W-R.1 |
| formula I.67 | B.66 | W-R.1 |
| formula I.68 | B.67 | W-R.1 |
| formula I.69 | B.68 | W-R.1 |
| formula I.70 | B.69 | W-R.1 |
| formula I.71 | B.70 | W-R.1 |
| formula I.72 | B.71 | W-R.1 |
| formula I.73 | B.72 | W-R.1 |
| formula I.74 | B.73 | W-R.1 |
| formula I.75 | B.74 | W-R.1 |
| formula I.76 | B.75 | W-R.1 |
| formula I.77 | B.76 | W-R.1 |
| formula I.78 | B.77 | W-R.1 |
| formula I.79 | B.78 | W-R.1 |
| formula I.80 | B.79 | W-R.1 |
| formula I.81 | B.80 | W-R.1 |
| formula I.82 | B.81 | W-R.1 |
| formula I.83 | B.82 | W-R.1 |
| formula I.84 | B.83 | W-R.1 |
| formula I.85 | B.84 | W-R.1 |
| formula I.86 | B.85 | W-R.1 |
| formula I.87 | B.86 | W-R.1 |
| formula I.88 | B.87 | W-R.1 |
| formula I.89 | B.88 | W-R.1 |
| formula I.90 | B.89 | W-R.1 |
| formula I.91 | B.90 | W-R.1 |
| formula I.92 | B.91 | W-R.1 |
| formula I.93 | B.92 | W-R.1 |
| formula I.94 | B.93 | W-R.1 |
| formula I.95 | B.94 | W-R.1 |
| formula I.96 | B.95 | W-R.1 |
| formula I.97 | B.96 | W-R.1 |
| formula I.98 | B.97 | W-R.1 |
| formula I.99 | B.98 | W-R.1 |
| formula I.100 | B.99 | W-R.1 |
| formula I.101 | B.100 | W-R.1 |
| formula I.102 | B.101 | W-R.1 |
| formula I.103 | B.102 | W-R.1 |
| formula I.104 | B.103 | W-R.1 |
| formula I.105 | B.104 | W-R.1 |
| formula I.106 | B.105 | W-R.1 |
| formula I.107 | B.106 | W-R.1 |
| formula I.108 | B.107 | W-R.1 |
| formula I.109 | B.108 | W-R.1 |
| formula I.110 | B.109 | W-R.1 |
| formula I.111 | B.110 | W-R.1 |
| formula I.112 | B.111 | W-R.1 |
| formula I.113 | B.112 | W-R.1 |
| formula I.114 | B.113 | W-R.1 |
| formula I.115 | B.114 | W-R.1 |
| formula I.116 | B.115 | W-R.1 |
| formula I.117 | B.116 | W-R.1 |
| formula I.118 | B.117 | W-R.1 |
| formula I.119 | B.118 | W-R.1 |
| formula I.120 | B.119 | W-R.1 |
| formula I.121 | B.120 | W-R.1 |
| formula I.122 | B.121 | W-R.1 |
| formula I.123 | B.122 | W-R.1 |
| formula I.124 | B.123 | W-R.1 |
| formula I.125 | B.124 | W-R.1 |
| formula I.126 | B.125 | W-R.1 |
| formula I.127 | B.126 | W-R.1 |
| formula I.128 | B.127 | W-R.1 |
| formula I.129 | B.128 | W-R.1 |
| formula I.130 | B.129 | W-R.1 |
| formula I.131 | B.130 | W-R.1 |
| formula I.132 | B.131 | W-R.1 |
| formula I.133 | B.132 | W-R.1 |
| formula I.134 | B.133 | W-R.1 |
| formula I.135 | B.134 | W-R.1 |
| formula I.136 | B.135 | W-R.1 |
| formula I.137 | B.136 | W-R.1 |
| formula I.138 | B.137 | W-R.1 |
| formula I.139 | B.138 | W-R.1 |
| formula I.140 | B.139 | W-R.1 |
| formula I.141 | B.140 | W-R.1 |
| formula I.142 | B.141 | W-R.1 |
| formula I.143 | B.142 | W-R.1 |
| formula I.144 | B.143 | W-R.1 |
| formula I.145 | B.144 | W-R.1 |
| formula I.146 | B.145 | W-R.1 |
| formula I.147 | B.146 | W-R.1 |
| formula I.148 | B.147 | W-R.1 |
| formula I.149 | B.148 | W-R.1 |
| formula I.150 | B.149 | W-R.1 |
| formula I.151 | B.150 | W-R.1 |
| formula I.152 | B.151 | W-R.1 |
| formula I.153 | B.152 | W-R.1 |
| formula I.154 | B.153 | W-R.1 |
| formula I.155 | B.154 | W-R.1 |
| formula I.156 | B.155 | W-R.1 |
| formula I.157 | B.156 | W-R.1 |
| formula I.158 | B.157 | W-R.1 |
| formula I.159 | B.158 | W-R.1 |
| formula I.160 | B.159 | W-R.1 |
| formula I.161 | B.160 | W-R.1 |
| formula I.162 | B.161 | W-R.1 |
| formula I.163 | B.162 | W-R.1 |
| formula I.164 | B.163 | W-R.1 |
| formula I.165 | B.164 | W-R.1 |
| formula I.166 | B.165 | W-R.1 |
| formula I.167 | B.166 | W-R.1 |
| formula I.168 | B.167 | W-R.1 |
| formula I.169 | B.168 | W-R.1 |
| formula I.170 | B.169 | W-R.1 |
| formula I.171 | B.170 | W-R.1 |
| formula I.172 | B.171 | W-R.1 |
| formula I.173 | B.172 | W-R.1 |
| formula I.174 | B.173 | W-R.1 |
| formula I.175 | B.174 | W-R.1 |
| formula I.176 | B.175 | W-R.1 |
| formula I.177 | B.176 | W-R.1 |
| formula I.178 | B.177 | W-R.1 |
| formula I.179 | B.178 | W-R.1 |
| formula I.180 | B.179 | W-R.1 |
| formula I.181 | B.180 | W-R.1 |
| formula I.182 | B.181 | W-R.1 |
| formula I.183 | B.182 | W-R.1 |
| formula I.184 | B.183 | W-R.1 |
| formula I.185 | B.184 | W-R.1 |
| formula I.186 | B.185 | W-R.1 |
| formula I.187 | B.186 | W-R.1 |
| formula I.188 | B.187 | W-R.1 |
| formula I.189 | B.188 | W-R.1 |
| formula I.190 | B.189 | W-R.1 |
| formula I.191 | B.190 | W-R.1 |
| formula I.192 | B.0 | W-R.21 |
| formula I.193 | B.1 | W-R.21 |
| formula I.194 | B.2 | W-R.21 |
| formula I.195 | B.3 | W-R.21 |
| formula I.196 | B.4 | W-R.21 |
| formula I.197 | B.5 | W-R.21 |
| formula I.198 | B.6 | W-R.21 |
| formula I.199 | B.7 | W-R.21 |
| formula I.200 | B.8 | W-R.21 |
| formula I.201 | B.9 | W-R.21 |
| formula I.202 | B.10 | W-R.21 |
| formula I.203 | B.11 | W-R.21 |
| formula I.204 | B.12 | W-R.21 |
| formula I.205 | B.13 | W-R.21 |
| formula I.206 | B.14 | W-R.21 |
| formula I.207 | B.15 | W-R.21 |
| formula I.208 | B.16 | W-R.21 |
| formula I.209 | B.17 | W-R.21 |
| formula I.210 | B.18 | W-R.21 |
| formula I.211 | B.19 | W-R.21 |
| formula I.212 | B.20 | W-R.21 |
| formula I.213 | B.21 | W-R.21 |
| formula I.214 | B.22 | W-R.21 |
| formula I.215 | B.23 | W-R.21 |
| formula I.216 | B.24 | W-R.21 |
| formula I.217 | B.25 | W-R.21 |
| formula I.218 | B.26 | W-R.21 |
| formula I.219 | B.27 | W-R.21 |
| formula I.220 | B.28 | W-R.21 |
| formula I.221 | B.29 | W-R.21 |
| formula I.222 | B.30 | W-R.21 |
| formula I.223 | B.31 | W-R.21 |
| formula I.224 | B.32 | W-R.21 |
| formula I.225 | B.33 | W-R.21 |
| formula I.226 | B.34 | W-R.21 |
| formula I.227 | B.35 | W-R.21 |
| formula I.228 | B.36 | W-R.21 |
| formula I.229 | B.37 | W-R.21 |
| formula I.230 | B.38 | W-R.21 |
| formula I.231 | B.39 | W-R.21 |
| formula I.232 | B.40 | W-R.21 |
| formula I.233 | B.41 | W-R.21 |
| formula I.234 | B.42 | W-R.21 |
| formula I.235 | B.43 | W-R.21 |
| formula I.236 | B.44 | W-R.21 |
| formula I.237 | B.45 | W-R.21 |
| formula I.238 | B.46 | W-R.21 |
| formula I.239 | B.47 | W-R.21 |
| formula I.240 | B.48 | W-R.21 |
| formula I.241 | B.49 | W-R.21 |
| formula I.242 | B.50 | W-R.21 |
| formula I.243 | B.51 | W-R.21 |
| formula I.244 | B.52 | W-R.21 |
| formula I.245 | B.53 | W-R.21 |
| formula I.246 | B.54 | W-R.21 |
| formula I.247 | B.55 | W-R.21 |
| formula I.248 | B.56 | W-R.21 |
| formula I.249 | B.57 | W-R.21 |
| formula I.250 | B.58 | W-R.21 |
| formula I.251 | B.59 | W-R.21 |
| formula I.252 | B.60 | W-R.21 |
| formula I.253 | B.61 | W-R.21 |
| formula I.254 | B.62 | W-R.21 |
| formula I.255 | B.63 | W-R.21 |
| formula I.256 | B.64 | W-R.21 |
| formula I.257 | B.65 | W-R.21 |
| formula I.258 | B.66 | W-R.21 |
| formula I.259 | B.67 | W-R.21 |
| formula I.260 | B.68 | W-R.21 |
| formula I.261 | B.69 | W-R.21 |
| formula I.262 | B.70 | W-R.21 |
| formula I.263 | B.71 | W-R.21 |
| formula I.264 | B.72 | W-R.21 |
| formula I.265 | B.73 | W-R.21 |
| formula I.266 | B.74 | W-R.21 |
| formula I.267 | B.75 | W-R.21 |
| formula I.268 | B.76 | W-R.21 |
| formula I.269 | B.77 | W-R.21 |
| formula I.270 | B.78 | W-R.21 |
| formula I.271 | B.79 | W-R.21 |
| formula I.272 | B.80 | W-R.21 |
| formula I.273 | B.81 | W-R.21 |
| formula I.274 | B.82 | W-R.21 |
| formula I.275 | B.83 | W-R.21 |
| formula I.276 | B.84 | W-R.21 |
| formula I.277 | B.85 | W-R.21 |
| formula I.278 | B.86 | W-R.21 |
| formula I.279 | B.87 | W-R.21 |
| formula I.280 | B.88 | W-R.21 |
| formula I.281 | B.89 | W-R.21 |
| formula I.282 | B.90 | W-R.21 |
| formula I.283 | B.91 | W-R.21 |
| formula I.284 | B.92 | W-R.21 |
| formula I.285 | B.93 | W-R.21 |
| formula I.286 | B.94 | W-R.21 |
| formula I.287 | B.95 | W-R.21 |
| formula I.288 | B.96 | W-R.21 |
| formula I.289 | B.97 | W-R.21 |
| formula I.290 | B.98 | W-R.21 |
| formula I.291 | B.99 | W-R.21 |
| formula I.292 | B.100 | W-R.21 |
| formula I.293 | B.101 | W-R.21 |
| formula I.294 | B.102 | W-R.21 |
| formula I.295 | B.103 | W-R.21 |
| formula I.296 | B.104 | W-R.21 |
| formula I.297 | B.105 | W-R.21 |
| formula I.298 | B.106 | W-R.21 |
| formula I.299 | B.107 | W-R.21 |
| formula I.300 | B.108 | W-R.21 |
| formula I.301 | B.109 | W-R.21 |
| formula I.302 | B.110 | W-R.21 |
| formula I.303 | B.111 | W-R.21 |
| formula I.304 | B.112 | W-R.21 |
| formula I.305 | B.113 | W-R.21 |
| formula I.306 | B.114 | W-R.21 |
| formula I.307 | B.115 | W-R.21 |
| formula I.308 | B.116 | W-R.21 |
| formula I.309 | B.117 | W-R.21 |
| formula I.310 | B.118 | W-R.21 |
| formula I.311 | B.119 | W-R.21 |
| formula I.312 | B.120 | W-R.21 |
| formula I.313 | B.121 | W-R.21 |
| formula I.314 | B.122 | W-R.21 |
| formula I.315 | B.123 | W-R.21 |
| formula I.316 | B.124 | W-R.21 |
| formula I.317 | B.125 | W-R.21 |
| formula I.318 | B.126 | W-R.21 |
| formula I.319 | B.127 | W-R.21 |
| formula I.320 | B.128 | W-R.21 |
| formula I.321 | B.129 | W-R.21 |
| formula I.322 | B.130 | W-R.21 |
| formula I.323 | B.131 | W-R.21 |
| formula I.324 | B.132 | W-R.21 |
| formula I.325 | B.133 | W-R.21 |
| formula I.326 | B.134 | W-R.21 |
| formula I.327 | B.135 | W-R.21 |
| formula I.328 | B.136 | W-R.21 |
| formula I.329 | B.137 | W-R.21 |
| formula I.330 | B.138 | W-R.21 |
| formula I.331 | B.139 | W-R.21 |
| formula I.332 | B.140 | W-R.21 |
| formula I.333 | B.141 | W-R.21 |
| formula I.334 | B.142 | W-R.21 |
| formula I.335 | B.143 | W-R.21 |
| formula I.336 | B.144 | W-R.21 |
| formula I.337 | B.145 | W-R.21 |
| formula I.338 | B.146 | W-R.21 |
| formula I.339 | B.147 | W-R.21 |
| formula I.340 | B.148 | W-R.21 |
| formula I.341 | B.149 | W-R.21 |
| formula I.342 | B.150 | W-R.21 |
| formula I.343 | B.151 | W-R.21 |
| formula I.344 | B.152 | W-R.21 |
| formula I.345 | B.153 | W-R.21 |
| formula I.346 | B.154 | W-R.21 |
| formula I.347 | B.155 | W-R.21 |
| formula I.348 | B.156 | W-R.21 |
| formula I.349 | B.157 | W-R.21 |
| formula I.350 | B.158 | W-R.21 |
| formula I.351 | B.159 | W-R.21 |
| formula I.352 | B.160 | W-R.21 |
| formula I.353 | B.161 | W-R.21 |
| formula I.354 | B.162 | W-R.21 |
| formula I.355 | B.163 | W-R.21 |
| formula I.356 | B.164 | W-R.21 |
| formula I.357 | B.165 | W-R.21 |
| formula I.358 | B.166 | W-R.21 |
| formula I.359 | B.167 | W-R.21 |
| formula I.360 | B.168 | W-R.21 |
| formula I.361 | B.169 | W-R.21 |
| formula I.362 | B.170 | W-R.21 |
| formula I.363 | B.171 | W-R.21 |
| formula I.364 | B.172 | W-R.21 |
| formula I.365 | B.173 | W-R.21 |
| formula I.366 | B.174 | W-R.21 |
| formula I.367 | B.175 | W-R.21 |
| formula I.368 | B.176 | W-R.21 |
| formula I.369 | B.177 | W-R.21 |
| formula I.370 | B.178 | W-R.21 |
| formula I.371 | B.179 | W-R.21 |
| formula I.372 | B.180 | W-R.21 |
| formula I.373 | B.181 | W-R.21 |
| formula I.374 | B.182 | W-R.21 |
| formula I.375 | B.183 | W-R.21 |
| formula I.376 | B.184 | W-R.21 |
| formula I.377 | B.185 | W-R.21 |
| formula I.378 | B.186 | W-R.21 |
| formula I.379 | B.187 | W-R.21 |
| formula I.380 | B.188 | W-R.21 |
| formula I.381 | B.189 | W-R.21 |
| formula I.382 | B.190 | W-R.21 |
| formula I.383 | B.0 | W-R.392 |
| formula I.384 | B.1 | W-R.392 |
| formula I.385 | B.2 | W-R.392 |
| formula I.386 | B.3 | W-R.392 |
| formula I.387 | B.4 | W-R.392 |
| formula I.388 | B.5 | W-R.392 |
| formula I.389 | B.6 | W-R.392 |
| formula I.390 | B.7 | W-R.392 |
| formula I.391 | B.8 | W-R.392 |
| formula I.392 | B.9 | W-R.392 |
| formula I.393 | B.10 | W-R.392 |
| formula I.394 | B.11 | W-R.392 |
| formula I.395 | B.12 | W-R.392 |
| formula I.396 | B.13 | W-R.392 |
| formula I.397 | B.14 | W-R.392 |
| formula I.398 | B.15 | W-R.392 |
| formula I.399 | B.16 | W-R.392 |
| formula I.400 | B.17 | W-R.392 |
| formula I.401 | B.18 | W-R.392 |
| formula I.402 | B.19 | W-R.392 |
| formula I.403 | B.20 | W-R.392 |
| formula I.404 | B.21 | W-R.392 |
| formula I.405 | B.22 | W-R.392 |
| formula I.406 | B.23 | W-R.392 |
| formula I.407 | B.24 | W-R.392 |
| formula I.408 | B.25 | W-R.392 |
| formula I.409 | B.26 | W-R.392 |
| formula I.410 | B.27 | W-R.392 |
| formula I.411 | B.28 | W-R.392 |
| formula I.412 | B.29 | W-R.392 |
| formula I.413 | B.30 | W-R.392 |
| formula I.414 | B.31 | W-R.392 |
| formula I.415 | B.32 | W-R.392 |
| formula I.416 | B.33 | W-R.392 |
| formula I.417 | B.34 | W-R.392 |
| formula I.418 | B.35 | W-R.392 |
| formula I.419 | B.36 | W-R.392 |
| formula I.420 | B.37 | W-R.392 |
| formula I.421 | B.38 | W-R.392 |
| formula I.422 | B.39 | W-R.392 |
| formula I.423 | B.40 | W-R.392 |
| formula I.424 | B.41 | W-R.392 |
| formula I.425 | B.42 | W-R.392 |
| formula I.426 | B.43 | W-R.392 |
| formula I.427 | B.44 | W-R.392 |
| formula I.428 | B.45 | W-R.392 |
| formula I.429 | B.46 | W-R.392 |
| formula I.430 | B.47 | W-R.392 |
| formula I.431 | B.48 | W-R.392 |
| formula I.432 | B.49 | W-R.392 |
| formula I.433 | B.50 | W-R.392 |
| formula I.434 | B.51 | W-R.392 |
| formula I.435 | B.52 | W-R.392 |
| formula I.436 | B.53 | W-R.392 |
| formula I.437 | B.54 | W-R.392 |
| formula I.438 | B.55 | W-R.392 |
| formula I.439 | B.56 | W-R.392 |
| formula I.440 | B.57 | W-R.392 |
| formula I.441 | B.58 | W-R.392 |
| formula I.442 | B.59 | W-R.392 |
| formula I.443 | B.60 | W-R.392 |
| formula I.444 | B.61 | W-R.392 |
| formula I.445 | B.62 | W-R.392 |
| formula I.446 | B.63 | W-R.392 |
| formula I.447 | B.64 | W-R.392 |
| formula I.448 | B.65 | W-R.392 |
| formula I.449 | B.66 | W-R.392 |
| formula I.450 | B.67 | W-R.392 |
| formula I.451 | B.68 | W-R.392 |
| formula I.452 | B.69 | W-R.392 |
| formula I.453 | B.70 | W-R.392 |
| formula I.454 | B.71 | W-R.392 |
| formula I.455 | B.72 | W-R.392 |
| formula I.456 | B.73 | W-R.392 |
| formula I.457 | B.74 | W-R.392 |
| formula I.458 | B.75 | W-R.392 |
| formula I.459 | B.76 | W-R.392 |
| formula I.460 | B.77 | W-R.392 |
| formula I.461 | B.78 | W-R.392 |
| formula I.462 | B.79 | W-R.392 |
| formula I.463 | B.80 | W-R.392 |
| formula I.464 | B.81 | W-R.392 |
| formula I.465 | B.82 | W-R.392 |
| formula I.466 | B.83 | W-R.392 |
| formula I.467 | B.84 | W-R.392 |
| formula I.468 | B.85 | W-R.392 |
| formula I.469 | B.86 | W-R.392 |
| formula I.470 | B.87 | W-R.392 |
| formula I.471 | B.88 | W-R.392 |
| formula I.472 | B.89 | W-R.392 |
| formula I.473 | B.90 | W-R.392 |
| formula I.474 | B.91 | W-R.392 |
| formula I.475 | B.92 | W-R.392 |
| formula I.476 | B.93 | W-R.392 |
| formula I.477 | B.94 | W-R.392 |
| formula I.478 | B.95 | W-R.392 |
| formula I.479 | B.96 | W-R.392 |
| formula I.480 | B.97 | W-R.392 |
| formula I.481 | B.98 | W-R.392 |
| formula I.482 | B.99 | W-R.392 |
| formula I.483 | B.100 | W-R.392 |
| formula I.484 | B.101 | W-R.392 |
| formula I.485 | B.102 | W-R.392 |
| formula I.486 | B.103 | W-R.392 |
| formula I.487 | B.104 | W-R.392 |
| formula I.488 | B.105 | W-R.392 |
| formula I.489 | B.106 | W-R.392 |
| formula I.490 | B.107 | W-R.392 |
| formula I.491 | B.108 | W-R.392 |
| formula I.492 | B.109 | W-R.392 |
| formula I.493 | B.110 | W-R.392 |
| formula I.494 | B.111 | W-R.392 |
| formula I.495 | B.112 | W-R.392 |
| formula I.496 | B.113 | W-R.392 |
| formula I.497 | B.114 | W-R.392 |
| formula I.498 | B.115 | W-R.392 |
| formula I.499 | B.116 | W-R.392 |
| formula I.500 | B.117 | W-R.392 |
| formula I.501 | B.118 | W-R.392 |
| formula I.502 | B.119 | W-R.392 |
| formula I.503 | B.120 | W-R.392 |
| formula I.504 | B.121 | W-R.392 |
| formula I.505 | B.122 | W-R.392 |
| formula I.506 | B.123 | W-R.392 |
| formula I.507 | B.124 | W-R.392 |
| formula I.508 | B.125 | W-R.392 |
| formula I.509 | B.126 | W-R.392 |
| formula I.510 | B.127 | W-R.392 |
| formula I.511 | B.128 | W-R.392 |
| formula I.512 | B.129 | W-R.392 |
| formula I.513 | B.130 | W-R.392 |
| formula I.514 | B.131 | W-R.392 |
| formula I.515 | B.132 | W-R.392 |
| formula I.516 | B.133 | W-R.392 |
| formula I.517 | B.134 | W-R.392 |
| formula I.518 | B.135 | W-R.392 |
| formula I.519 | B.136 | W-R.392 |
| formula I.520 | B.137 | W-R.392 |
| formula I.521 | B.138 | W-R.392 |
| formula I.522 | B.139 | W-R.392 |
| formula I.523 | B.140 | W-R.392 |
| formula I.524 | B.141 | W-R.392 |
| formula I.525 | B.142 | W-R.392 |
| formula I.526 | B.143 | W-R.392 |
| formula I.527 | B.144 | W-R.392 |
| formula I.528 | B.145 | W-R.392 |
| formula I.529 | B.146 | W-R.392 |
| formula I.530 | B.147 | W-R.392 |
| formula I.531 | B.148 | W-R.392 |
| formula I.532 | B.149 | W-R.392 |
| formula I.533 | B.150 | W-R.392 |
| formula I.534 | B.151 | W-R.392 |
| formula I.535 | B.152 | W-R.392 |
| formula I.536 | B.153 | W-R.392 |
| formula I.537 | B.154 | W-R.392 |
| formula I.538 | B.155 | W-R.392 |
| formula I.539 | B.156 | W-R.392 |
| formula I.540 | B.157 | W-R.392 |
| formula I.541 | B.158 | W-R.392 |
| formula I.542 | B.159 | W-R.392 |
| formula I.543 | B.160 | W-R.392 |
| formula I.544 | B.161 | W-R.392 |
| formula I.545 | B.162 | W-R.392 |
| formula I.546 | B.163 | W-R.392 |
| formula I.547 | B.164 | W-R.392 |
| formula I.548 | B.165 | W-R.392 |
| formula I.549 | B.166 | W-R.392 |
| formula I.550 | B.167 | W-R.392 |
| formula I.551 | B.168 | W-R.392 |
| formula I.552 | B.169 | W-R.392 |
| formula I.553 | B.170 | W-R.392 |
| formula I.554 | B.171 | W-R.392 |
| formula I.555 | B.172 | W-R.392 |
| formula I.556 | B.173 | W-R.392 |
| formula I.557 | B.174 | W-R.392 |
| formula I.558 | B.175 | W-R.392 |
| formula I.559 | B.176 | W-R.392 |
| formula I.560 | B.177 | W-R.392 |
| formula I.561 | B.178 | W-R.392 |
| formula I.562 | B.179 | W-R.392 |
| formula I.563 | B.180 | W-R.392 |
| formula I.564 | B.181 | W-R.392 |
| formula I.565 | B.182 | W-R.392 |
| formula I.566 | B.183 | W-R.392 |
| formula I.567 | B.184 | W-R.392 |
| formula I.568 | B.185 | W-R.392 |
| formula I.569 | B.186 | W-R.392 |
| formula I.570 | B.187 | W-R.392 |
| formula I.571 | B.188 | W-R.392 |
| formula I.572 | B.189 | W-R.392 |
| formula I.573 | B.190 | W-R.392 |
| formula I.574 | B.0 | W-R.405 |
| formula I.575 | B.1 | W-R.405 |
| formula I.576 | B.2 | W-R.405 |
| formula I.577 | B.3 | W-R.405 |
| formula I.578 | B.4 | W-R.405 |
| formula I.579 | B.5 | W-R.405 |
| formula I.580 | B.6 | W-R.405 |
| formula I.581 | B.7 | W-R.405 |
| formula I.582 | B.8 | W-R.405 |
| formula I.583 | B.9 | W-R.405 |
| formula I.584 | B.10 | W-R.405 |
| formula I.585 | B.11 | W-R.405 |
| formula I.586 | B.12 | W-R.405 |
| formula I.587 | B.13 | W-R.405 |
| formula I.588 | B.14 | W-R.405 |
| formula I.589 | B.15 | W-R.405 |
| formula I.590 | B.16 | W-R.405 |
| formula I.591 | B.17 | W-R.405 |
| formula I.592 | B.18 | W-R.405 |
| formula I.593 | B.19 | W-R.405 |
| formula I.594 | B.20 | W-R.405 |
| formula I.595 | B.21 | W-R.405 |
| formula I.596 | B.22 | W-R.405 |
| formula I.597 | B.23 | W-R.405 |
| formula I.598 | B.24 | W-R.405 |
| formula I.599 | B.25 | W-R.405 |
| formula I.600 | B.26 | W-R.405 |
| formula I.601 | B.27 | W-R.405 |
| formula I.602 | B.28 | W-R.405 |
| formula I.603 | B.29 | W-R.405 |
| formula I.604 | B.30 | W-R.405 |
| formula I.605 | B.31 | W-R.405 |
| formula I.606 | B.32 | W-R.405 |
| formula I.607 | B.33 | W-R.405 |
| formula I.608 | B.34 | W-R.405 |
| formula I.609 | B.35 | W-R.405 |
| formula I.610 | B.36 | W-R.405 |
| formula I.611 | B.37 | W-R.405 |
| formula I.612 | B.38 | W-R.405 |
| formula I.613 | B.39 | W-R.405 |
| formula I.614 | B.40 | W-R.405 |
| formula I.615 | B.41 | W-R.405 |
| formula I.616 | B.42 | W-R.405 |
| formula I.617 | B.43 | W-R.405 |
| formula I.618 | B.44 | W-R.405 |
| formula I.619 | B.45 | W-R.405 |
| formula I.620 | B.46 | W-R.405 |
| formula I.621 | B.47 | W-R.405 |
| formula I.622 | B.48 | W-R.405 |
| formula I.623 | B.49 | W-R.405 |
| formula I.624 | B.50 | W-R.405 |
| formula I.625 | B.51 | W-R.405 |
| formula I.626 | B.52 | W-R.405 |
| formula I.627 | B.53 | W-R.405 |
| formula I.628 | B.54 | W-R.405 |
| formula I.629 | B.55 | W-R.405 |
| formula I.630 | B.56 | W-R.405 |
| formula I.631 | B.57 | W-R.405 |
| formula I.632 | B.58 | W-R.405 |
| formula I.633 | B.59 | W-R.405 |
| formula I.634 | B.60 | W-R.405 |
| formula I.635 | B.61 | W-R.405 |
| formula I.636 | B.62 | W-R.405 |
| formula I.637 | B.63 | W-R.405 |
| formula I.638 | B.64 | W-R.405 |
| formula I.639 | B.65 | W-R.405 |
| formula I.640 | B.66 | W-R.405 |
| formula I.641 | B.67 | W-R.405 |
| formula I.642 | B.68 | W-R.405 |
| formula I.643 | B.69 | W-R.405 |
| formula I.644 | B.70 | W-R.405 |
| formula I.645 | B.71 | W-R.405 |
| formula I.646 | B.72 | W-R.405 |
| formula I.647 | B.73 | W-R.405 |
| formula I.648 | B.74 | W-R.405 |
| formula I.649 | B.75 | W-R.405 |
| formula I.650 | B.76 | W-R.405 |
| formula I.651 | B.77 | W-R.405 |
| formula I.652 | B.78 | W-R.405 |
| formula I.653 | B.79 | W-R.405 |
| formula I.654 | B.80 | W-R.405 |
| formula I.655 | B.81 | W-R.405 |
| formula I.656 | B.82 | W-R.405 |
| formula I.657 | B.83 | W-R.405 |
| formula I.658 | B.84 | W-R.405 |
| formula I.659 | B.85 | W-R.405 |
| formula I.660 | B.86 | W-R.405 |
| formula I.661 | B.87 | W-R.405 |
| formula I.662 | B.88 | W-R.405 |
| formula I.663 | B.89 | W-R.405 |
| formula I.664 | B.90 | W-R.405 |
| formula I.665 | B.91 | W-R.405 |
| formula I.666 | B.92 | W-R.405 |
| formula I.667 | B.93 | W-R.405 |
| formula I.668 | B.94 | W-R.405 |
| formula I.669 | B.95 | W-R.405 |
| formula I.670 | B.96 | W-R.405 |
| formula I.671 | B.97 | W-R.405 |
| formula I.672 | B.98 | W-R.405 |
| formula I.673 | B.99 | W-R.405 |
| formula I.674 | B.100 | W-R.405 |
| formula I.675 | B.101 | W-R.405 |
| formula I.676 | B.102 | W-R.405 |
| formula I.677 | B.103 | W-R.405 |
| formula I.678 | B.104 | W-R.405 |
| formula I.679 | B.105 | W-R.405 |
| formula I.680 | B.106 | W-R.405 |
| formula I.681 | B.107 | W-R.405 |
| formula I.682 | B.108 | W-R.405 |
| formula I.683 | B.109 | W-R.405 |
| formula I.684 | B.110 | W-R.405 |
| formula I.685 | B.111 | W-R.405 |
| formula I.686 | B.112 | W-R.405 |
| formula I.687 | B.113 | W-R.405 |
| formula I.688 | B.114 | W-R.405 |
| formula I.689 | B.115 | W-R.405 |
| formula I.690 | B.116 | W-R.405 |
| formula I.691 | B.117 | W-R.405 |
| formula I.692 | B.118 | W-R.405 |
| formula I.693 | B.119 | W-R.405 |
| formula I.694 | B.120 | W-R.405 |
| formula I.695 | B.121 | W-R.405 |
| formula I.696 | B.122 | W-R.405 |
| formula I.697 | B.123 | W-R.405 |
| formula I.698 | B.124 | W-R.405 |
| formula I.699 | B.125 | W-R.405 |
| formula I.700 | B.126 | W-R.405 |
| formula I.701 | B.127 | W-R.405 |
| formula I.702 | B.128 | W-R.405 |
| formula I.703 | B.129 | W-R.405 |
| formula I.704 | B.130 | W-R.405 |
| formula I.705 | B.131 | W-R.405 |
| formula I.706 | B.132 | W-R.405 |
| formula I.707 | B.133 | W-R.405 |
| formula I.708 | B.134 | W-R.405 |
| formula I.709 | B.135 | W-R.405 |
| formula I.710 | B.136 | W-R.405 |
| formula I.711 | B.137 | W-R.405 |
| formula I.712 | B.138 | W-R.405 |
| formula I.713 | B.139 | W-R.405 |
| formula I.714 | B.140 | W-R.405 |
| formula I.715 | B.141 | W-R.405 |
| formula I.716 | B.142 | W-R.405 |
| formula I.717 | B.143 | W-R.405 |
| formula I.718 | B.144 | W-R.405 |
| formula I.719 | B.145 | W-R.405 |
| formula I.720 | B.146 | W-R.405 |
| formula I.721 | B.147 | W-R.405 |
| formula I.722 | B.148 | W-R.405 |
| formula I.723 | B.149 | W-R.405 |
| formula I.724 | B.150 | W-R.405 |
| formula I.725 | B.151 | W-R.405 |
| formula I.726 | B.152 | W-R.405 |
| formula I.727 | B.153 | W-R.405 |
| formula I.728 | B.154 | W-R.405 |
| formula I.729 | B.155 | W-R.405 |
| formula I.730 | B.156 | W-R.405 |
| formula I.731 | B.157 | W-R.405 |
| formula I.732 | B.158 | W-R.405 |
| formula I.733 | B.159 | W-R.405 |
| formula I.734 | B.160 | W-R.405 |
| formula I.735 | B.161 | W-R.405 |
| formula I.736 | B.162 | W-R.405 |
| formula I.737 | B.163 | W-R.405 |
| formula I.738 | B.164 | W-R.405 |
| formula I.739 | B.165 | W-R.405 |
| formula I.740 | B.166 | W-R.405 |
| formula I.741 | B.167 | W-R.405 |
| formula I.742 | B.168 | W-R.405 |
| formula I.743 | B.169 | W-R.405 |
| formula I.744 | B.170 | W-R.405 |
| formula I.745 | B.171 | W-R.405 |
| formula I.746 | B.172 | W-R.405 |
| formula I.747 | B.173 | W-R.405 |
| formula I.748 | B.174 | W-R.405 |
| formula I.749 | B.175 | W-R.405 |
| formula I.750 | B.176 | W-R.405 |
| formula I.751 | B.177 | W-R.405 |
| formula I.752 | B.178 | W-R.405 |
| formula I.753 | B.179 | W-R.405 |
| formula I.754 | B.180 | W-R.405 |
| formula I.755 | B.181 | W-R.405 |
| formula I.756 | B.182 | W-R.405 |
| formula I.757 | B.183 | W-R.405 |
| formula I.758 | B.184 | W-R.405 |
| formula I.759 | B.185 | W-R.405 |
| formula I.760 | B.186 | W-R.405 |
| formula I.761 | B.187 | W-R.405 |
| formula I.762 | B.188 | W-R.405 |
| formula I.763 | B.189 | W-R.405 |
| formula I.764 | B.190 | W-R.405 |
| formula I.765 | B.0 | W-R.418 |
| formula I.766 | B.1 | W-R.418 |
| formula I.767 | B.2 | W-R.418 |
| formula I.768 | B.3 | W-R.418 |
| formula I.769 | B.4 | W-R.418 |
| formula I.770 | B.5 | W-R.418 |
| formula I.771 | B.6 | W-R.418 |
| formula I.772 | B.7 | W-R.418 |
| formula I.773 | B.8 | W-R.418 |
| formula I.774 | B.9 | W-R.418 |
| formula I.775 | B.10 | W-R.418 |
| formula I.776 | B.11 | W-R.418 |
| formula I.777 | B.12 | W-R.418 |
| formula I.778 | B.13 | W-R.418 |
| formula I.779 | B.14 | W-R.418 |
| formula I.780 | B.15 | W-R.418 |
| formula I.781 | B.16 | W-R.418 |
| formula I.782 | B.17 | W-R.418 |
| formula I.783 | B.18 | W-R.418 |
| formula I.784 | B.19 | W-R.418 |
| formula I.785 | B.20 | W-R.418 |
| formula I.786 | B.21 | W-R.418 |
| formula I.787 | B.22 | W-R.418 |
| formula I.788 | B.23 | W-R.418 |
| formula I.789 | B.24 | W-R.418 |
| formula I.790 | B.25 | W-R.418 |
| formula I.791 | B.26 | W-R.418 |
| formula I.792 | B.27 | W-R.418 |
| formula I.793 | B.28 | W-R.418 |
| formula I.794 | B.29 | W-R.418 |
| formula I.795 | B.30 | W-R.418 |
| formula I.796 | B.31 | W-R.418 |
| formula I.797 | B.32 | W-R.418 |
| formula I.798 | B.33 | W-R.418 |
| formula I.799 | B.34 | W-R.418 |
| formula I.800 | B.35 | W-R.418 |
| formula I.801 | B.36 | W-R.418 |
| formula I.802 | B.37 | W-R.418 |
| formula I.803 | B.38 | W-R.418 |
| formula I.804 | B.39 | W-R.418 |
| formula I.805 | B.40 | W-R.418 |
| formula I.806 | B.41 | W-R.418 |
| formula I.807 | B.42 | W-R.418 |
| formula I.808 | B.43 | W-R.418 |
| formula I.809 | B.44 | W-R.418 |
| formula I.810 | B.45 | W-R.418 |
| formula I.811 | B.46 | W-R.418 |
| formula I.812 | B.47 | W-R.418 |
| formula I.813 | B.48 | W-R.418 |
| formula I.814 | B.49 | W-R.418 |
| formula I.815 | B.50 | W-R.418 |
| formula I.816 | B.51 | W-R.418 |
| formula I.817 | B.52 | W-R.418 |
| formula I.818 | B.53 | W-R.418 |
| formula I.819 | B.54 | W-R.418 |
| formula I.820 | B.55 | W-R.418 |
| formula I.821 | B.56 | W-R.418 |
| formula I.822 | B.57 | W-R.418 |
| formula I.823 | B.58 | W-R.418 |
| formula I.824 | B.59 | W-R.418 |
| formula I.825 | B.60 | W-R.418 |
| formula I.826 | B.61 | W-R.418 |
| formula I.827 | B.62 | W-R.418 |
| formula I.828 | B.63 | W-R.418 |
| formula I.829 | B.64 | W-R.418 |
| formula I.830 | B.65 | W-R.418 |
| formula I.831 | B.66 | W-R.418 |
| formula I.832 | B.67 | W-R.418 |
| formula I.833 | B.68 | W-R.418 |
| formula I.834 | B.69 | W-R.418 |
| formula I.835 | B.70 | W-R.418 |
| formula I.836 | B.71 | W-R.418 |
| formula I.837 | B.72 | W-R.418 |
| formula I.838 | B.73 | W-R.418 |
| formula I.839 | B.74 | W-R.418 |
| formula I.840 | B.75 | W-R.418 |
| formula I.841 | B.76 | W-R.418 |
| formula I.842 | B.77 | W-R.418 |
| formula I.843 | B.78 | W-R.418 |
| formula I.844 | B.79 | W-R.418 |
| formula I.845 | B.80 | W-R.418 |
| formula I.846 | B.81 | W-R.418 |
| formula I.847 | B.82 | W-R.418 |
| formula I.848 | B.83 | W-R.418 |
| formula I.849 | B.84 | W-R.418 |
| formula I.850 | B.85 | W-R.418 |
| formula I.851 | B.86 | W-R.418 |
| formula I.852 | B.87 | W-R.418 |
| formula I.853 | B.88 | W-R.418 |
| formula I.854 | B.89 | W-R.418 |
| formula I.855 | B.90 | W-R.418 |
| formula I.856 | B.91 | W-R.418 |
| formula I.857 | B.92 | W-R.418 |
| formula I.858 | B.93 | W-R.418 |
| formula I.859 | B.94 | W-R.418 |
| formula I.860 | B.95 | W-R.418 |
| formula I.861 | B.96 | W-R.418 |
| formula I.862 | B.97 | W-R.418 |
| formula I.863 | B.98 | W-R.418 |
| formula I.864 | B.99 | W-R.418 |
| formula I.865 | B.100 | W-R.418 |
| formula I.866 | B.101 | W-R.418 |
| formula I.867 | B.102 | W-R.418 |
| formula I.868 | B.103 | W-R.418 |
| formula I.869 | B.104 | W-R.418 |
| formula I.870 | B.105 | W-R.418 |
| formula I.871 | B.106 | W-R.418 |
| formula I.872 | B.107 | W-R.418 |
| formula I.873 | B.108 | W-R.418 |
| formula I.874 | B.109 | W-R.418 |
| formula I.875 | B.110 | W-R.418 |
| formula I.876 | B.111 | W-R.418 |
| formula I.877 | B.112 | W-R.418 |
| formula I.878 | B.113 | W-R.418 |
| formula I.879 | B.114 | W-R.418 |
| formula I.880 | B.115 | W-R.418 |
| formula I.881 | B.116 | W-R.418 |
| formula I.882 | B.117 | W-R.418 |
| formula I.883 | B.118 | W-R.418 |
| formula I.884 | B.119 | W-R.418 |
| formula I.885 | B.120 | W-R.418 |
| formula I.886 | B.121 | W-R.418 |
| formula I.887 | B.122 | W-R.418 |
| formula I.888 | B.123 | W-R.418 |
| formula I.889 | B.124 | W-R.418 |
| formula I.890 | B.125 | W-R.418 |
| formula I.891 | B.126 | W-R.418 |
| formula I.892 | B.127 | W-R.418 |
| formula I.893 | B.128 | W-R.418 |
| formula I.894 | B.129 | W-R.418 |
| formula I.895 | B.130 | W-R.418 |
| formula I.896 | B.131 | W-R.418 |
| formula I.897 | B.132 | W-R.418 |
| formula I.898 | B.133 | W-R.418 |
| formula I.899 | B.134 | W-R.418 |
| formula I.900 | B.135 | W-R.418 |
| formula I.901 | B.136 | W-R.418 |
| formula I.902 | B.137 | W-R.418 |
| formula I.903 | B.138 | W-R.418 |
| formula I.904 | B.139 | W-R.418 |
| formula I.905 | B.140 | W-R.418 |
| formula I.906 | B.141 | W-R.418 |
| formula I.907 | B.142 | W-R.418 |
| formula I.908 | B.143 | W-R.418 |
| formula I.909 | B.144 | W-R.418 |
| formula I.910 | B.145 | W-R.418 |
| formula I.911 | B.146 | W-R.418 |
| formula I.912 | B.147 | W-R.418 |
| formula I.913 | B.148 | W-R.418 |
| formula I.914 | B.149 | W-R.418 |
| formula I.915 | B.150 | W-R.418 |
| formula I.916 | B.151 | W-R.418 |
| formula I.917 | B.152 | W-R.418 |
| formula I.918 | B.153 | W-R.418 |
| formula I.919 | B.154 | W-R.418 |
| formula I.920 | B.155 | W-R.418 |
| formula I.921 | B.156 | W-R.418 |
| formula I.922 | B.157 | W-R.418 |
| formula I.923 | B.158 | W-R.418 |
| formula I.924 | B.159 | W-R.418 |
| formula I.925 | B.160 | W-R.418 |
| formula I.926 | B.161 | W-R.418 |
| formula I.927 | B.162 | W-R.418 |
| formula I.928 | B.163 | W-R.418 |
| formula I.929 | B.164 | W-R.418 |
| formula I.930 | B.165 | W-R.418 |
| formula I.931 | B.166 | W-R.418 |
| formula I.932 | B.167 | W-R.418 |
| formula I.933 | B.168 | W-R.418 |
| formula I.934 | B.169 | W-R.418 |
| formula I.935 | B.170 | W-R.418 |
| formula I.936 | B.171 | W-R.418 |
| formula I.937 | B.172 | W-R.418 |
| formula I.938 | B.173 | W-R.418 |
| formula I.939 | B.174 | W-R.418 |
| formula I.940 | B.175 | W-R.418 |
| formula I.941 | B.176 | W-R.418 |
| formula I.942 | B.177 | W-R.418 |
| formula I.943 | B.178 | W-R.418 |
| formula I.944 | B.179 | W-R.418 |
| formula I.945 | B.180 | W-R.418 |
| formula I.946 | B.181 | W-R.418 |
| formula I.947 | B.182 | W-R.418 |
| formula I.948 | B.183 | W-R.418 |
| formula I.949 | B.184 | W-R.418 |
| formula I.950 | B.185 | W-R.418 |
| formula I.951 | B.186 | W-R.418 |
| formula I.952 | B.187 | W-R.418 |
| formula I.953 | B.188 | W-R.418 |
| formula I.954 | B.189 | W-R.418 |
| formula I.955 | B.190 | W-R.418 |

The compounds of the present invention can be e.g. prepared from the corresponding amines AM-I by the synthetic route outlined in the following scheme:

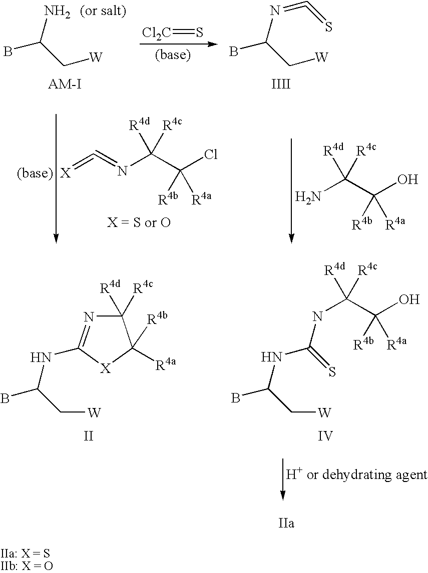

According to the method outlined in the scheme, an amine AM-I is converted to the corresponding isothiocyanate III by conventional means, e.g. by reacting AM-I with thiophosgene (see e.g. Houben-Weyl, E4, “Methoden der Organischen Chemie”, chapter IIc, pp. 837-842, Georg Thieme Verlag 1983). The isothiocyanate III is then reacted with aminoethanol, thereby obtaining the thiourea IV.

The reaction of the aminoethanol with isothiocyanate III can be performed in accordance with standard methods of organic chemistry, see e.g. Biosci. Biotech. Biochem. 56 (7), 1062-65 (1992).

The thus obtained thioureas IV can be cyclized by conventional means thereby obtaining the desired thiazoline compound of the formula IIa. Cyclization of compound IV can be achieved e.g. under acid catalysis or under dehydrating conditions e.g. by Mitsunobu's reaction (see Tetrahedron Letters 1999, 40, 3125-3128).

According to the method outlined in the scheme, an amine AM-I can also be converted to azolines of formula IIa or IIb by reaction with 2-chloroethylisothiocyanate or 2-chloroethylisocyanate e.g. as described in Bioorg. Med. Chem. Lett. 1994, 4, 2317-22 and subsequent cyclization in the presence or absence of base or under the conditions described below.

Amines as AM-I are known in the art or can in general be prepared by methods familiar to an organic chemist and well known in the art e.g. J. Chinese Chem. Soc. 2003, 50, 109-114, Synthesis 1976, 593-595, J. Med. Chem. 1995, 38, 10, 1600-1607, J. Org. Chem. 1962, 1970-1972, or for example by reduction of appropriate precursors derived by condensation of appropriate nitro methane compounds with aldehydes as demonstrated below (preparation examples) according to similar methods described e.g. in J. Am. Chem. Soc. 1934, 1556-1558 or J. Org. Chem. 1956, 1118-1119. Additionally, amines AM-I can be prepared by alkylation of appropriate imines and subsequent hydrolysis as described below (preparation examples) according to similar methods described e.g. in Tet. Asymm. 1995, 6, 1225-1228. Alternatively, amines AM-I can also be obtained by addition of appropriate methylenic lithio or magnesium halide reagents in the presence or not of copper or zinc additives to appropriate imines as described below (Preparation examples) according to similar methods described e.g. in Org. Lett. 2001, 3, 1841-1844.

2-Chloroethyl isothiocyanate (CAS-reg.-no.: 6099-88-3) and 2-chloroethylisocyanate (CAS-reg.-no.: 1943-83-5) are commercially available.

Compounds of the general formula V or VI can be obtained by reaction with a suitable electrophile as outlined in the following scheme e.g. an alkylating or acylating reagent R^{5/6}-LG (LG=leaving group; e.g. Cl, Br, I, OSO₂R, OCO₂R, wherein R is C₁-C₄-alkyl), e.g. as described in WO 2005063724.

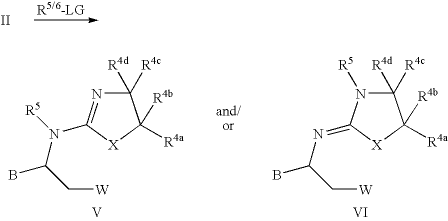

As a further alternative, the compounds of the present invention can also be prepared starting from amines AM-II via aminothiazolines or aminooxazolines VII. A further conversion with an alkylating or acylating reagent R^{5/6}-LG provides derivatised aminothiazolines or aminooxazolines VIII and/or IX. Those are subsequently converted with appropriate halomethyl heteroaromatic reagents, as outlined in the following scheme and below (preparation examples):

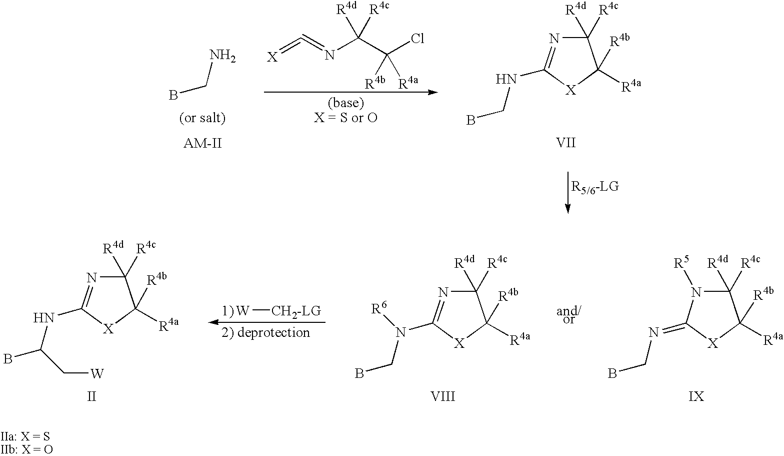

The meaning of the substituents B, W, R^{4a-d}, R⁵ and R⁶ in the schemes correspond to the definition given above.

### Pests

Due to their excellent activity, the compounds of the formula I may be used for controlling animal pests, in particular selected harmful insects, arachnids and nematodes.

The compounds of the formula I and the pestidicidal compositions comprising them are therefore effective agents for controlling animal pests, selected from insects, arachnids and nematodes. Animal pests controlled by the compounds of formula I include for example:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;
beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria;
dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;
thrips (Thysanoptera), e.g. Dichromothrips corbetti, Frankliniella fusca, Frankliniella occidentalis, Frankliniella tritici, Scirtothrips citri, Thrips oryzae, Thrips palmi and Thrips tabaci;
hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta;
heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;
homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus persicae, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Sogatella furcifera Trialeurodes vaporariorum, Toxoptera aurantiiand, and Viteus vitifolii;
termites (Isoptera), e.g. Calotermes flavicollis, Leucotermes flavipes, Reticulitermes flavipes, Reticulitermes lucifugus und Termes natalensis;
orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;
Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;
Siphonatera, e.g. Xenopsylla cheopsis, Ceratophyllus spp;

The compositions and compounds of formula I are useful for the control of nematodes, especially plant parasitic nematodes such as root knot nematodes, Meloidogyne hapla,

Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

In a preferred embodiment of the invention the compounds of formula I are used for controlling insects or arachnids, in particular insects of the orders Lepidoptera, Coleoptera and Homoptera and arachnids of the order Acarina. The compounds of the formula I according to the present invention are particularly useful for controlling insects of the order Lepidoptera and Homoptera.

### Formulations

For use in a method according to the present invention, the compounds of formula I can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula I according to the present invention.

Accordingly, the invention further provides e.g. an agriculturally composition for combating such animal pests, which comprises such an amount of at least one compound of the formula I or at least an agriculturally useful salt of I and at least one inert liquid and/or solid agronomically acceptable carrier that it has a pesticidal action and, if desired, at least one surfactant.

Such a composition may contain a single active compound of the formula I, or the enantiomers thereof or a mixture of several active compounds of formula I according to the present invention. The composition according to the present invention may comprise an individual isomer or mixtures of isomers as well as individual tautomers or mixtures of tautomers.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones (N-methyl-pyrrolidone (NMP), N-octylpyrrolidone NOP), acetates (glycol diacetate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters,

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example dichlorophen und benzyl alcohol hemiformal

Suitable thickeners are compounds, which confer a pseudoplastic flow behavior to the formulation, i.e. high viscosity at rest and low viscosity in the agitated stage. Mention may be made, in this context, for example, of commercial thickeners based on polysaccharides, such as Xanthan Gum® (Kelzan® from Kelco), Rhodopol® 23 (Rhone Poulenc) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compound of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1% per weight.

The active ingredients may also be used successfully in the ultra-low-volume process (ULV), it being possible to apply formulations comprising over 95% by weight of active ingredient, or even to apply the active ingredient without additives.

The following are examples of formulations:

1. Products for dilution with water. For seed treatment purposes, such products may be applied to the seed diluted or undiluted.

### A) Water-Soluble Concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active compound is obtained.

### B) Dispersible Concentrates (DC)

20 parts by weight of the active compound is dissolved in 70 parts by weight of cyclohexanone with addition of 10 parts by weight of a dispersant, for example polyvinylpyrrolidone. Dilution with water gives a dispersion, whereby a formulation with 20% (w/w) of active compounds is obtained.

### C) Emulsifiable Concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

### D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

### E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

### F) Water-Dispersible Granules and Water-Soluble Granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

### G) Water-Dispersible Powders and Water-Soluble Powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

### H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.

2. Products to be applied undiluted for foliar applications. For seed treatment purposes, such products may be applied to the seed diluted or undiluted.

### I) Dustable Powders (DP, DS)

5 parts by weight of the active compound are ground finely and mixed intimately with 95 parts by weight of finely divided kaolin. This gives a dustable product having 5% (w/w) of active compound.

### J) Granules (GR, FG, GG, MG)

0.5 part by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

### K) ULV Solutions (UL)

10 parts by weight of the active compound is dissolved in 90 parts by weight of an organic solvent, for example xylene. This gives a product having 10% (w/w) of active compound, which is applied undiluted for foliar use.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds and compositions of the present invention compounds I may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These additional agents can be admixed with the agents used according to the invention in a weight ratio of 1:10 to 10:1. Mixing the compounds I or the compositions comprising them in the use form as pesticides with other pesticides frequently results in a broader pesticidal spectrum of action.

The following list M of pesticides together with which the compounds according to the invention can be used and with which potential synergistic effects might be produced, is intended to illustrate the possible combinations, but not to impose any limitation:

M.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
M.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
M.3. Pyrethroids: allethrin, bifenthrin, cyfluthrin, cyhalothrin, cyphenothrin, cypermethrin, alpha-cypermethrin, beta-cypermethri n, zeta-cypermethrin, deltamethrin, empenthrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
M.4. Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
M.5. Nicotinic receptor agonists/antagonists compounds: clothianidin, dinotefuran, imidacloprid, thiamethoxam, nitenpyram, acetamiprid, thiacloprid and AKD-1022;
M.6. GABA antagonist compounds: acetoprole, endosulfan, ethiprole, fipronil, vaniliprole, pyrafluprole, pyriprole, the phenylpyrazole compound of formula γ²

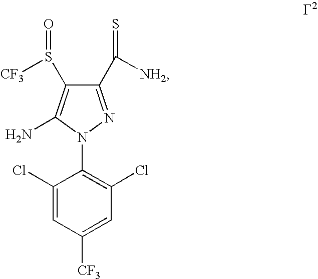

M.7. Macrocyclic lactone insecticides: abamectin, emamectin, milbemectin, lepimectin, spinosad
M.8. METI I compounds: fenazaquin, pyridaben, tebufenpyrad, tolfenpyrad, flufenerim;
M.9. METI II and III compounds: acequinocyl, fluacyprim, hydramethylnon;
M.10. Uncoupler compounds: chlorfenapyr;
M.11. Oxidative phosphorylation inhibitor compounds: cyhexatin, diafenthiuron, fenbutatin oxide, propargite;
M.12. Moulting disruptor compounds: cyromazine;
M.13. Mixed Function Oxidase inhibitor compounds: piperonyl butoxide;
M.14. Sodium channel blocker compounds: indoxacarb, metaflumizone,
M.15. Various: amitraz, benclothiaz, bifenazate, cartap, flonicamid, pyridalyl, pymetrozine, sulfur, thiocyclam, flubendiamide, cyenopyrafen, flupyrazofos, cyflumetofen, amidoflumet, pyrifluquinazon, N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, anthranilamide compounds as chlorantraniliprole or the compound of formula Γ⁵

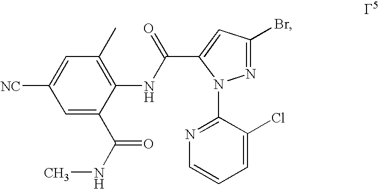

and malononitrile compounds as described in JP 2002 284608, WO 02/89579, WO 02/90320, WO 02/90321, WO 04/06677, WO 04/20399, JP 2004 99597, WO 05/68423, WO 05/68432, or WO 05/63694, especially the malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, and CF₃CF₂CH₂C(CN)₂CH₂(CF₂)₃CF₂H.

The commercially available compounds of the group M may be found in The Pesticide Manual, 13^{th} Edition, British Crop Protection Council (2003) among other publications. Thioamides of formula Γ² and their preparation have been described in WO 98/28279. Lepimectin is known from Agro Project, PJB Publications Ltd, November 2004. Benclothiaz and its preparation have been described in EP-A1 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54,1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180. The aminoquinazolinone compound of formula Γ⁴ has been described in EP A 109 7932.

Anthranilamides as the one of formula γ⁵ or as chloranthraniliprole and their preparations have been described in WO 01/70671; WO 02/48137; WO 03/24222, WO 03/15518, WO 04/67528; WO 04/33468; and WO 05/118552. The malononitrile compounds C F₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, and CF₃CF₂CH₂C(CN)₂CH₂(CF₂)₃CF₂H have been described in WO 05/63694.

Fungicidal mixing partners are those selected from the group F consisting of

F.1 acylalanines such as benalaxyl, metalaxyl, ofurace, oxadixyl;
F.2 amine derivatives such as aldimorph, dodine, dodemorph, fenpropimorph, fenpropidin, guazatine, iminoctadine, spiroxamin, tridemorph;
F.3 anilinopyrimidines such as pyrimethanil, mepanipyrim or cyrodinyl;
F.4 antibiotics such as cycloheximid, griseofulvin, kasugamycin, natamycin, polyoxin or streptomycin;
F.5 azoles such as bitertanol, bromoconazole, cyproconazole, difenoconazole, dinitroconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol;
F.6 dicarboximides such as iprodion, myclozolin, procymidon, vinclozolin;
F.7 dithiocarbamates such as ferbam, nabam, maneb, mancozeb, metam, metiram, propineb, polycarbamate, thiram, ziram, zineb;
F.8 heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine;
F.9 copper fungicides such as Bordeaux mixture, copper acetate, copper oxychloride, basic copper sulfate;
F.10 nitrophenyl derivatives such as binapacryl, dinocap, dinobuton, nitrophthalisopropyl;
F.11 phenylpyrroles such as fenpiclonil or fludioxonil;
F.12 strobilurins such as azoxystrobin, dimoxystrobin, fluoxastrobin, kresoxim-methyl, metominostrobin, orysastrobin, picoxystrobin or trifloxystrobin;
F.13 sulfenic acid derivatives such as captafol, captan, dichlofluanid, folpet, tolylfluanid;
F.14 cinnemamides and analogs such as dimethomorph, flumetover or flumorph;
F.15 sulfur, and other fungicides such as acibenzolar-S-methyl, benthiavalicarb, carpropamid, chlorothalonil, cyflufenamid, cymoxanil, dazomet, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofosmethyl, quintozene, zoxamid.

### Applications

The animal pest, i.e. the insects, arachnids and nematodes, the plant, soil or water in which the plant is growing can be contacted with the present compound(s) I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

Moreover, animal pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula I are effective through both contact (via soil, glass, wall, bed net, carpet, plant parts or animal parts), and ingestion (bait, or plant part).

For use against ants, termites, wasps, flies, mosquitos, crickets, or cockroaches, compounds of formula I are preferably used in a bait composition. The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

For use in treating crop plants, the rate of application of the active ingredients of this invention may be in the range of 0.1 g to 4000 g per hectare, desirably from 25 g to 600 g per hectare, more desirably from 50 g to 500 g per hectare.

### Seed Treatment

The compounds of formula I are also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants. Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of 1, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### Animal Health

The compounds of formula I or the enantiomers or veterinarily acceptable salts thereof are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or the enantiomers or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or the enantiomers or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula I or the enantiomers or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I are suitable for combating endo- and ectoparasites in and on animals.

Compounds of formula I or the enantiomers or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or the enantiomers or veterinarily acceptable salts thereof and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
Bugs (Heteropterida): Cimex lectularius, Cimex hemipterus, Reduvius senilis, Triatoma spp., Rhodnius ssp., Panstrongylus spp. and Arilus critatus,
Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp,
Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of the compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and compositions containing them for combating ticks is a further preferred embodiment of the present invention.

The compounds of formula I also are especially useful for combating endoparasites (roundworms nematoda, thorny headed worms and planarians).

Administration can be carried out both prophylactically and therapeutically. Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day. Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents which are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids,
fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;
ampholytic surfactants such as di-sodium N-lauryl-p-iminodipropionate or lecithin; anionic surfactants, such as sodium lauryl sulfate, fatty alcohol ether sulfates, mono/dialkyl polyglycol ether orthophosphoric acid ester monoethanolamine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

The present invention is now illustrated in further detail by the following examples.

### COMPOUND EXAMPLES

The compounds of formula I according to the invention as well as intermediates were characterized by coupled High Performance Liquid Chromatography/mass spectroscopy (HPLC/MS), by NMR or by their melting points. HPLC column: RP-18 column (Chromolith Speed ROD from Merck KgaA, Germany). Elution: acetonitrile+0.1% trifluoroacetic acid (TFA)/water+0.1% TFA in a ratio of from 5:95 to 95:5 in 5 minutes at 40° C. MS Quadrupol electrospray ionisation, 80 V (positive modus).

TABLE C

| | | Physical property |
|---|---|---|
| | | (m.p. [° C.], retention time tᵣ [min], |
| | | mass spectrum m/z (HPLC/MS) or |
| n° of | | chemical shift (¹H-NMR [CDCl₃, δ |
| compound | Formula | ppm]) |
| 1. | 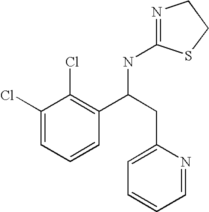 | m.p. = 160-162° C. |
| 2. | 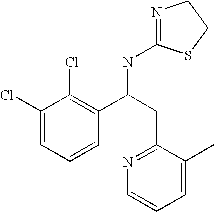 | ¹H-NMR: 2.16 (s, 3H), 3.12-3.34 (m,4H), 3.67-3.80 (m, 2H), 5.44-5.47(m, 1H), 7.04-7.09 (m, 2H), 7.14 (d,1H), 7.31-7.37 (m, 2H), 8.40 (d, 1H) |
| 3. | 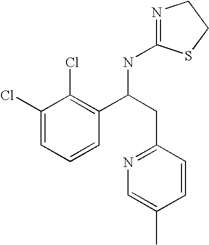 | ¹H-NMR: 2.30 (s, 3H), 3.02 (dd, 1H),3.06-3.22 (m, 2H), 3.30 (dd, 1 H),3.79-3.92 (m, 2H), 5.40-5.44 (m,1H), 6.83 (d, 1H), 7.02-7.07 (m, 2H),7.29-7.34 (m, 2H), 8.36 (s, 1H) |
| 4. | 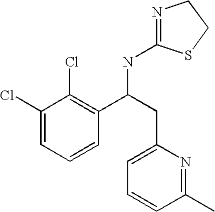 | ¹H-NMR: 2.58 (s, 3H), 3.00 (dd, 1H),3.19-3.31 (m, 3H), 3.79-3.92 (m,2H), 5.37-5.40 (m, 1 H), 6.78 (d, 1 H),6.98-7.10 (m, 3H), 7.31 (dd, 1H),7.40 (t, 1 H) |
| 5. | 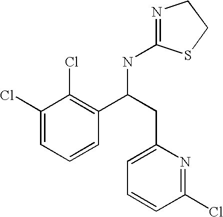 | tᵣ = 2.72 minm/z = 386 (M + 1) |
| 6. | 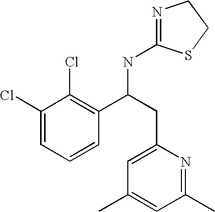 | m.p. = 111-113° C. |
| 7. | 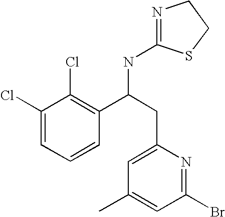 | ¹H-NMR: 2.48 (s, 3H), 2.83 (dd, 1H),3.07 (dd, 1H), 3.21-3.24 (m, 2H),3.72-3.75 (m, 2H), 5.11-5.16 (m,1H), 6.93 (s, 1H), 7.13 (s, 1H), 7.17-7.30 (m, 2H), 7.40 (d, 1H) |
| 8. | 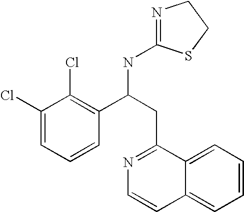 | m.p. = 117-121° C. |
| 9. | 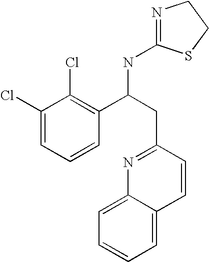 | m.p. = 129-131° C. |
| 10. | 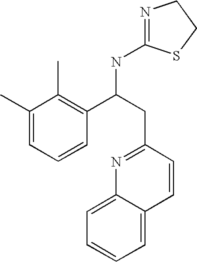 | m.p. = 139-141° C. |
| 11. | 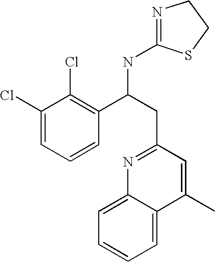 | m.p. = 126-128° C. |
| 12. | 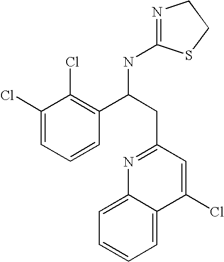 | m.p. = 71-73° C. |
| 13. | 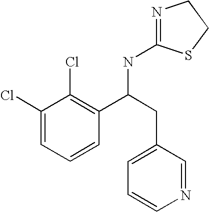 | m.p. = 190-193° C. |
| 14. | 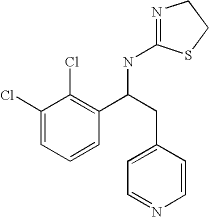 | m.p. = 224-225° C. |
| 15. | 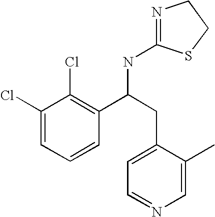 | m.p. = 157-159° C. |
| 16. | 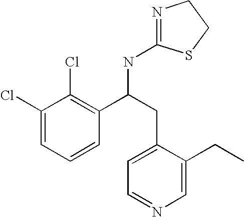 | m.p. = 132-134° C. |
| 17. | 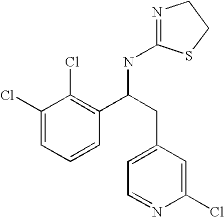 | tᵣ = 2.80 minm/z = 386 (M + 1) |
| 18. | 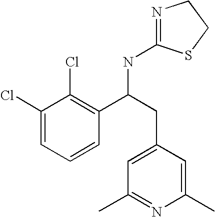 | ¹H-NMR: 2.47 (s, 6H), 2.79 (dd, 1H),3.07 (dd, 1H), 3.20-3.25 (m, 2H),3.77-3.87 (m, 2H), 5.21-5.24 (m,1H), 6.78 (s, 2H), 7.17-7.29 (m, 2H),7.40 (d, 1H) |
| 19. | 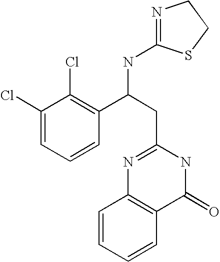 | m.p. = 293-294° C. |
| 20. | 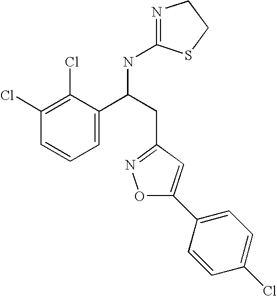 | tᵣ = 3.26 minm/z = 452 (M + 1) |
| 21. | 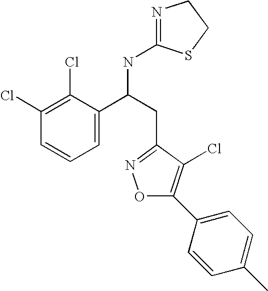 | tᵣ = 3.46 minm/z = 466 (M + 1) |
| 22. | 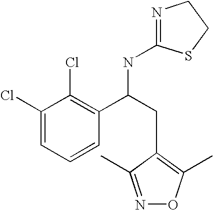 | m.p. = 188-194° C. |
| 23. | 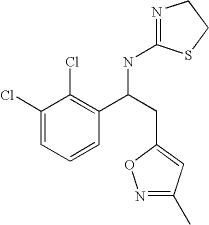 | m.p. = 128-130° C. |
| 24. | 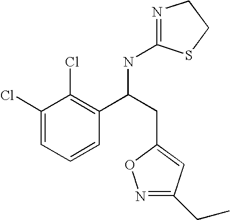 | ¹H-NMR: 1.22 (t, 3H), 2.63 (q, 2H),3.17 (dd, 1H), 3.21-3.32 (m, 3H),3.77-3.82 (m, 2H), 5.32-5.37 (m,1H), 5.81 (s, 1H), 7.11-7.24 (m, 2H),7.37 (dd, 1H) |
| 25. | 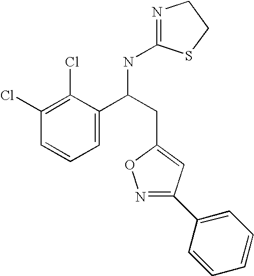 | tᵣ = 2.98 minm/z = 418 (M + 1) |
| 26. | 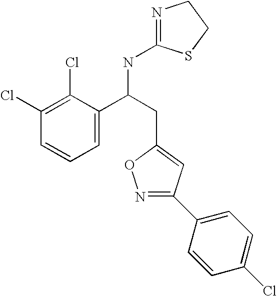 | tᵣ = 3.24 minm/z = 452 (M + 1) |
| 27. | 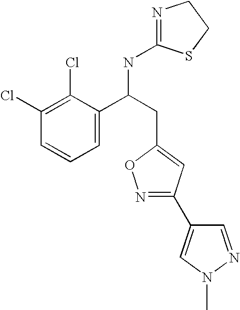 | tᵣ = 2.43 minm/z = 422 (M + 1) |
| 28. | 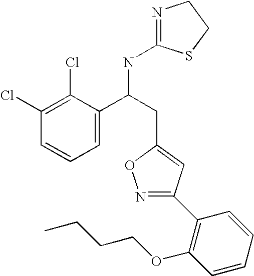 | tᵣ = 3.49 minm/z = 490 (M + 1) |
| 29. | 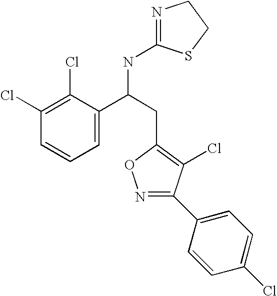 | tᵣ = 3.46 minm/z = 486 (M + 1) |
| 30. | 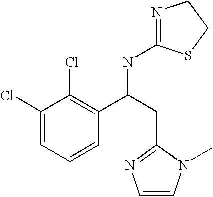 | m.p. = 195-196° C. |
| 31. | 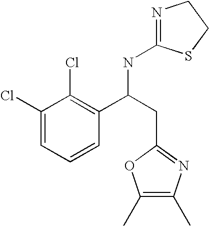 | m.p. = 146-149° C. |
| 32. | 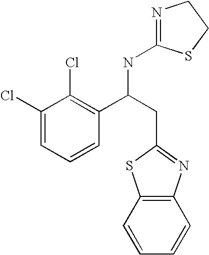 | m.p. = 165-166° C. |
| 33. | 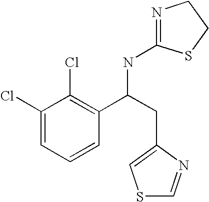 | m.p. = 154-156° C. |
| 34. | 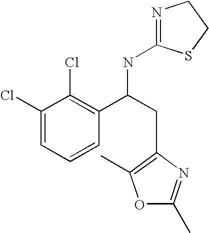 | tᵣ = 2.56 minm/z = 370 (M + 1) |
| 35. | 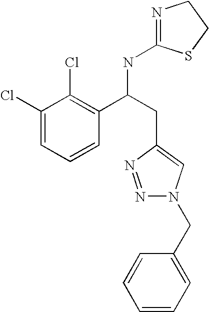 | m.p. = 74-89° C. |
| 36. | 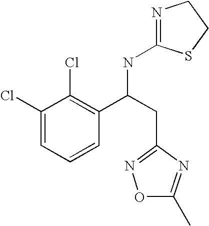 | m.p. = 137-141° C. |
| 37. | 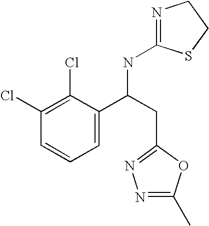 | tᵣ = 2.342 minm/z = 357 (M + 1) |
| 38. | 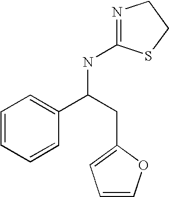 | ¹H-NMR = 3.2-3.4 (m), 3.85 (mc),4.95 (mc), 5.95 (d), 6.20 (d), 7.05-7.4 (m). |
| 39. | 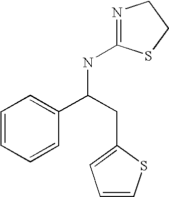 | ¹H-NMR = 3.25 (t), 3.35 (mc), 3.9 (t),4.85 (mc), 6.7-7.4 (m) |

### P. PREPARATION EXAMPLES

The compounds of formula I according to the present invention can be prepared by different methods and via different intermediate products as described in the following:

### P.I Method A

### P.I.1. Preparation Level 1: Ethylamine Derivatives as Intermediate Compounds

### P.I.1.a) Step 1: Preparation of 2-(2-nitro-2-phenyl-vinyl)-thiophene

To a solution of nitromethyl benzene (1.00 g; cf. J. Org. Chem. 2004, 6907-6908) and thiophene-2-carbaldehyde (0.42 g) in toluene (10 ml) was added potassium fluoride (42 mg) and dimethylamine hydrochloride (0.60 g) and the solution was heated under reflux in a Dean Stark trap for 4 h. The solvent was evaporated and the residue dissolved in dichloromethane, washed with water and dried over sodium sulphate. Evaporation of the solvent yielded a crude product (1.35 g), which was further purified by tituration in a 1:1 mixture of hexane and ethyl acetate giving 0.96 g of the product.

### P.I.1.a) Step 2: Preparation of 1-phenyl-2-thiophen-2-yl-ethylamine

To a slurry of lithium aluminium hydride (0.75 g) in anhydrous tetrahydrofuran (15 ml) was added at 0° C. conc. sulphuric acid (0.49 g) portionwise (exothermic reaction) within 30 min and stirring continued for an additional 30 min at the same temperature. A solution of 2-(2-nitro-2-phenyl-vinyl)-thiophene (0.96 g) in tetrahydrofuran (17 ml) was added within 30 min and stirring continued for 1 h at 0° C. and then for 3 h at room temperature. The reaction mixture was quenched with a diluted sodium hydroxide solution and the precipitates removed by filtration. The filtrate was dried over sodium sulphate and concentrated in vacuo to yield 1.02 g of crude 1-phenyl-2-thiophen-2-yl-ethylamine that was used without further purification for the next step.

### P.I.1.b) Step 1: Preparation of benzhydrylidene-(2,3-dichloro-benzyl)-amine

A mixture of 2,3-dichlorobenzylamine (23.5 g) and benzophenone imine (26.6 g) in dichloromethane (100 mL) was stirred overnight at room temperature and subsequently refluxed for 4 h. The mixture was concentrated in vacuo, the residue triturated in methanol and filtered to deliver the title compound (39.1 g, 82 wt. %).

### P.I.1.b) Step 2: Preparation of benzhydrylidene-[1-(2,3-dichloro-phenyl)-2-(3-ethylisoxazol-5-yl)-ethyl]-amine

A mixture of benzhydrylidene-(2,3-dichloro-benzyl)-amine (1.50 g), 5-chloromethyl-3-ethylisoxazole (1.28 g), benzyltriethylammonium chloride (100 mg), sodium hydroxide (1.06 g), potassium carbonate (2.44 g) and sodium iodide (66 mg) in acetonitrile (50 mL) was refluxed for 3 h. The mixture was then diluted with diethyl ether, washed with water, dried over sodium sulfate and concentrated in vacuo. The residue was chromatographed to deliver the title compound (1.58 g, 76 wt. %).

### P.I.1.b) Step 3: Preparation of 1-(2,3-dichloro-phenyl)-2-(3-ethyl-isoxazol-5-yl)-ethylamine

A 10% aqueous solution of HCl (12 mL) was added to a solution of benzhydrylidene-[1-(2,3-dichloro-phenyl)-2-(3-ethylisoxazol-5-yl)-ethyl]-amine (500 mg) in diethyl ether (10 mL) and the mixture stirred overnight. The phases were then separated, the aqueous layer was washed with ethyl acetate, basified to pH 8 with saturated aqueous sodium bicarbonate and extracted with dichloromethane. The extracts were dried over sodium sulfate and concentrated to deliver the title compound (270 mg, 81 wt. %).

### P.I.1.c) Preparation of 1-(2,3-dichloro-phenyl)-2-(4-methyl-quinolin-2-yl)ethylamine

A 1 M solution of lithium bis(trimethylsilyl)amide in tetrahydrofuran (11 mL) was added dropwise to a solution of 2,3-dichlorobenzaldehyde (1.75 g) in tetrahydrofuran (25 mL) at 0° C. The mixture was stirred for 15 min at 0° C. and then 1 h at room temperature. In the meantime, a 1.5 M solution of n-butyllithium in n-hexane (7 mL) was added dropwise to a solution of 2,4-dimethylquinoline (1.57 g) in tetrahydrofuran (12 mL) at −78° C. After stirring for 30 min at −78° C., 30 min at 0° C. and 15 min at room temperature, the solution of trimethylsilylimine was added dropwise at −78° C., the resulting mixture stirred for 2.5 h, poured into ice-water and extracted with ethyl acetate. The combined organic extracts were washed with water, dried over sodium sulphate, concentrated in vacuo and the residue chromatographed to deliver the title compound (2.54 g, 77 wt. %).

### P.I.2. Preparation Level 2: Thiourea Derivative as Intermediate Compound

### P.I.2.a) Preparation of 1-(2-hydroxy-ethyl)-3-(1-phenyl-2-thiophen-2-yl-ethyl) thiourea

To a solution of thiophosgene (0.70 g) in dichloromethane was added a solution of potassium carbonate (1.70 g) in water (6 ml). To this mixture was added a solution of 1-phenyl-2-thiophen-2-yl-ethylamine (1.00 g) in dichloromethane dropwise at room temperature. After stirring for 5 h the mixture was washed with water and the organic phase dried over sodium sulphate and concentrated in vacuo to yield the crude isothiocyanate, which was dissolved in dichloromethane (70 ml) and treated with ethanolamine (0.24 g). This reaction mixture was stirred for 3 h, then concentrated in vacuo and the remainder purified by column chromatography to yield 0.92 g of 1-(2-hydroxy-ethyl)-3-(1-phenyl-2-thiophen-2-yl-ethyl)-thiourea, mp. 109-112° C.

### P.I.3. Preparation Level 3: Compound of Formula I According to the Invention

### P.I.3.a)-1 Preparation of (4,5-dihydro-thiazol-2-yl)-(1-phenyl-2-thiophen-2-yl-ethyl)-amine (Compound Example 39 of Table C)

To a solution of 1-(2-hydroxy-ethyl)-3-(1-phenyl-2-thiophen-2-yl-ethyl)-thiourea (0.71 g) and triphenylphosphine (0.91 g) in tetrahydrofuran (24 ml) was added dropwise at room temperature a solution of diisopropylazodicarboxylate (0.60 g) in tetrahydrofuran (7 ml). After stirring for 1 h the reaction mixture was concentrated in vacuo and the remainder purified by column chromatography to yield 0.45 g of the product.

### P.I.3.a)-2 Preparation of (4,5-dihydro-thiazol-2-yl)-(1-phenyl-2-furan-2-yl-ethyl)-amine (Compound Example 38 of Table C)

To a solution of 1-(2-hydroxy-ethyl)-3-(1-phenyl-2-furan-2-yl-ethyl)-thiourea (0.72 g) (prepared in a similar way as described above for 1-(2-hydroxy-ethyl)-3-(1-phenyl-2-thiophen-2-yl-ethyl)-thiourea) and diisopropylethylamine (0.45 g) in propionitrile (7 ml) was added cyanomethyl-trimethyl-phosphonium iodide (J. Med. Chem. 2004, 6055-6069). The reaction mixture was heated up to 90° C. for 6 h and stirred at room temperature overnight. Ethyl acetate was added and washed with diluted potassium carbonate solution (5 wt. % in water), water and dried over sodium sulphate. The remainder was purified by column chromatography to yield the product in 0.37 g.

### P.II. Method B

P.II.1. Preparation Level 1: Thiazolidine-3-carboxylic acid tert-butyl ester as Intermediate Compound

### P.II.1. Step 1: Preparation of (2,3-dichloro-benzyl)-(4,5-dihydro-thiazol-2-yl)-amine

A solution of 2-chloroethyl isothiocyanate (3.45 g) in diethyl ether (20 mL) was added dropwise to a solution of 2,3-dichlorobenzylamine (5.00 g) in diethyl ether (80 mL) at 0° C. and the mixture was allowed to warm up to room temperature over 3 h. 1 M aqueous NaOH (100 mL) was then added and the mixture stirred for 1 h. The layers were then separated, the organic phase washed with water, dried over sodium sulfate and concentrated in vacuo. The residue was chromatographed to deliver the title compound (7.79 g, 94 wt. %).

### P.II.1. Step 2: Preparation of 2-(2,3-dichloro-benzylimino)-thiazolidine-3-carboxylic acid tert-butyl ester

Di-tert-butyl dicarbonate (9.77 g) was added to a solution of (2,3-dichloro-benzyl)-(4,5-dihydro-thiazol-2-yl)-amine (7.79 g) in dichloromethane (100 mL) at 0° C. and the mixture was allowed to warm to room temperature overnight. The solution was washed with water, dried over sodium sulfate, concentrated in vacuo and the residue chromatographed to deliver the title compound (6.84 g, 67 wt. %).

### P.II.1. Step 3: Preparation of 2-[1-(2,3-dichloro-phenyl)-2-pyridin-3-yl-ethylimino]-thiazolidine-3-carboxylic acid tert-butyl ester

A solution of 1.3 M sec-butyllithium in cyclohexane (1.7 mL) was added dropwise to a mixture of potassium tert-butoxide (247 mg) in tetrahydrofuran (6 mL) at −78° C. and the mixture stirred for 15 min. A solution of 2-(2,3-dichloro-benzylimino)-thiazolidine-3-carboxylic acid tert-butyl ester (361 mg) in tetrahydrofuran (4 mL) was then added dropwise and the mixture stirred for 2 h. A solution of 3-chloromethyl-pyridine (281 mg) in tetrahydrofuran (2 mL) was then added dropwise and the mixture stirred for 30 min at −78° C. and then 30 min at −30° C. Saturated aqueous ammonium chloride was then added at 0° C. and the mixture extracted with ethyl acetate. The combined organic extracts were dried over sodium sulfate, concentrated and the residue chromatographed

to deliver the title compound (380 mg, 75 wt. %).

### P.II.2. Preparation Level 2: Compound of Formula I According to the Invention

### P.II.2. Preparation of [1-(2,3-dichloro-phenyl)-2-(2,5-dimethyl-oxazol-4-yl)-ethyl]-4,5-dihydro-thiazol-2-yl)-amine (Compound Example 34 of Table C)

Trifluoroacetic acid (10 μL) was added dropwise to a solution of 2-[1-(2,3-dichlorophenyl)-2-(2,5-dimethyl-oxazol-4-yl)-ethylimino]-thiazolidine-3-carboxylic acid tert-butyl ester (65 mg) (prepared analogously as described above) in tetrahydrofuran (1 mL) at 0° C. and the mixture allowed to warm to room temperature over 1 h. The title compound was obtained in >80% purity according to HPLC-MS [tᵣ=2.56 min, m/z=370 (M+1)].

### B. BIOLOGICAL EXAMPLES

### Examples of Action Against Pests

The action of the compounds I against pests was demonstrated by the following experiments:

B.1 Cotton Aphid (Aphis gossypii)

The active compounds were formulated in 50:50 acetone:water and 100 ppm Kinetica™ surfactant.

Cotton plants in the cotyledon stage (variety ‘Delta Pine’) are infested with approximately 100 laboratory-reared aphids by placing infested leaf sections on top of the test plants. The leaf sections are removed after 24 hr. The cotyledons of the intact plants are dipped into gradient solutions of the test compound. Aphid mortality on the treated plants, relative to mortality on check plants, is determined after 5 days.

In this test, the compound examples 1, 3,4,6,8,9,24,38 and 39 of table C showed at 300 ppm over 80% mortality in comparison with untreated controls.

B.2 Green Peach Aphid (Myzus persicae)

The active compounds were formulated in 50:50 acetone:water and 100 ppm Kinetica™ surfactant.

Pepper plants in the 2^{nd} leaf-pair stage (variety ‘California Wonder’) are infested with approximately 40 laboratory-reared aphids by placing infested leaf sections on top of the test plants. The leaf sections are removed after 24 hr. The leaves of the intact plants are dipped into gradient solutions of the test compound. Aphid mortality on the treated plants, relative to mortality on check plants, is determined after 5 days.

In this test, the compound examples 1, 3,4,6,8,9,13,24,38 and 39 of table C showed at 300 ppm over 80% mortality in comparison with untreated controls.

B.3 Cowpea Aphid (aphis craccivora)

The active compounds were formulated in 50:50 acetone:water. Potted cowpea plants colonized with 100-150 aphids of various stages were sprayed after the pest population has been recorded. Population reduction was recorded after 24, 72, and 120 hours.

In this test, compounds examples 1, 3,4,6,8,9,23 and 31 of table C showed at 300 ppm over 80% mortality in comparison with untreated controls.

B.4 Vetch Aphid (Megoura viciae)

The active compounds were formulated in 1:3 DMSO:water. Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated twice.

In this test, compound examples 1-4,6,8 and 9 of table C showed at 2500 ppm over 80% mortality in comparison with untreated controls.

1. A compound of the formula (I): 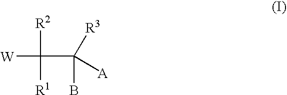
wherein
R¹, R², R³ are selected from hydrogen, C₁-C₆-alkyl, C₁-C₆-haloalkyl, C₃-C₆-cycloalkyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
phenyl or benzyl, each unsubstituted or substituted with any combination of one to five halogen, one to three C₁-C₆-alkyl, C₁-C₆-haloalkyl, C₁-C₆-alkylthio, C₁-C₆-haloalkylthio, C₁-C₆-alkoxy or C₁-C₆-haloalkoxy groups;
A is a radical of the formulae A¹ or A²; 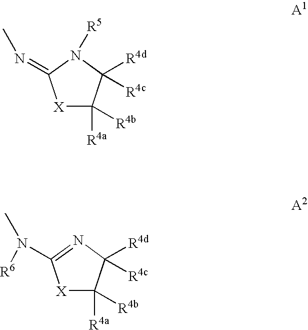
wherein
X is sulfur or oxygen;
R^{4a}, R^{4b}, R^{4c}, R^{4d} are each independently selected from one another from hydrogen, halogen, C₁-C₆-alkyl, C₁-C₆-haloalkyl, C₁-C₆-alkylamino, C₁-C₆-alkoxy or C₃-C₆-cycloalkyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
R⁵, R⁶, R⁹ are each independently selected from one another from hydrogen, cyano, nitro, formyl, C(═O)R^{5c}, C(═O)R^{6c}, C(═O)R^{9c}, C₁-C₆-alkyl, C₂-C₆-alkenyl, C₂-C₆-alkinyl, C₃-C₈-cycloalkyl, C₁-C₆-alkoxy, (C₁-C₆-alkoxy)methylen, C₁-C₆-alkylsulfinyl, C₁-C₆-alkylsulfenyl, C₁-C₆-alkylsulfonyl wherein the carbon atoms in the aliphatic radicals of the aforementioned groups may carry any combination of 1, 2 or 3 radicals, independently of one another selected from the group consisting of halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy and C₁-C₆-alkylthio; C(O)NR^{5a}R^{5b}, C(O)NR^{6a}R^{6b}, C(O)NR^{9a}R^{9b}, (SO₂)NR^{5a}R^{5b}, (SO₂)NR^{6a}R^{6b}, (SO₂)NR^{9a}R^{9b},
phenyl, phenyloxy or benzyl, each of the last three mentioned radicals may be unsubstituted or substituted with 1 to 5 radicals, independently of one another selected from the group consisting of one to five halogen radicals, one to three C₁-C₆-alkyl, one to three C₁-C₆-haloalkyl, one to three C₁-C₆-alkylthio, one to three C₁-C₆-haloalkylthio, one to three C₁-C₆-alkoxy and one to three C₁-C₆-haloalkoxy radicals;
and wherein R^{5a}, R^{6a}, R^{9a}, R^{5b}, R^{6b}, R^{9b}, R^{5c}, R^{6c} and R^{9c} are defined as below;
B is an optionally substituted phenyl ring system
W is a 5- to 6-membered heteroaromatic ring which may contain 1 to 4
heteroatoms selected from oxygen, nitrogen and sulfur,
wherein the heteroaromatic ring may optionally be fused to a ring selected from phenyl and a 5- to 6-membered saturated, partially unsaturated or aromatic heterocyclic ring which may contain 1 to 3 heteroatoms selected from oxygen, nitrogen and sulfur,
and wherein the 5- to 6-membered heteroaromatic ring or the respective fused ring systems may be unsubstituted or optionally substituted by R⁹ and/or any combination of 1 to 4 groups R⁸: 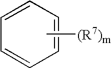
wherein n is 0, 1, 2, 3, or 4;
and R⁸ is defined as below;
and R⁹ is defined as above
and wherein
R⁸ is selected from halogen, OH, SH, NH₂, SO₃H, COOH, cyano, azido, nitro, formyl, CONH₂, CSNH₂, CH═N—OH, CH═N—O—(C₁-C₆)-alkyl, C(═O)R^{8c}, C(═S)R^{8c}, C₁-C₆-alkyl, C₂-C₆-alkenyl, C₂-C₆-alkynyl, C₃-C₈-cycloalkyl, C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkylthio, C₂-C₆-alkenylthio, C₂-C₆-alkynylthio, C₁-C₆-alkylsulfonyl, C₂-C₆-alkenylsulfonyl, C₂-C₆-alkynylsulfonyl, (C₁-C₆-alkyl)carbonyl, (C₂-C₆-alkenyl)-carbonyl, (C₂-C₆-alkynyl)-carbonyl, C₁-C₆-alkoxy, C₂-C₆-alkenyloxy, C₂-C₆-alkynyloxy, (C₁-C₆-alkoxy)carbonyl, (C₂-C₆-alkenyloxy)carbonyl, (C₂-C₆-alkynyloxy)-carbonyl, (C₁-C₆-alkyl)carbonyloxy, (C₂-C₆-alkenyl-)carbonyl-oxy, (C₂-C₆-alkynyl)carbonyloxy, (C₁-C₆-alkyl)carbonyl-amino, (C₂-C₆-alkenyl)carbonyl-amino, (C₂-C₆-alkynyl)carbonyl-amino, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
C(O)NR^{8a}R^{8b}, (SO₂)NR^{8a}R^{8b}; wherein R^{8a}, R^{8b} and R^{8c} are defined as below, or
a radical Y—Ar or a radical Y-Cy, wherein
Y is a single bond, O, S, NH, C₁-C₆-alkandiyl or C₁-C₆-alkandiyloxy;
Ar is phenyl, naphthyl or a mono- or bicyclic 5- to 10-membered heteroaromatic ring, which contains 1, 2, 3 or 4 heteroatoms selected from 1 or 2 oxygen, 1 or 2 sulfur and 1 to 3 nitrogen atoms as ring members, wherein Ar is unsubstituted or may carry any combination of one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
Cy is C₃-C₁₂-cycloalkyl, which is unsubstituted or substituted with one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
and wherein
R^{5a}, R^{6a}, R^{8a} and R^{5b}, R^{6b}, R^{8b}
are each independently selected from one another from hydrogen, C₁-C₆-alkyl, C₁-C₆-haloalkyl, C₂-C₆-alkenyl, or C₂-C₆-alkynyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
and R^{5c}, R^{6c}, R^{8c} and R^{9c}
are selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl or heteroaryl, which can be a mono- or bicyclic 5 to 10 membered heteroaromatic ring, which contains 1, 2, 3 or 4 heteroatoms selected from O, S and N
or the enantiomers, diastereomers or salts thereof.

2. An 1-(Azolin-2-yl)-amino-1-phenyl-2-hetaryl-ethane compound according to claim 1, wherein B is selected from: 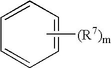
wherein
m is 0, 1, 2, 3, 4 or 5;
R⁷ is selected from halogen, OH, SH, NH₂, SO₃H, COOH, cyano, azido, nitro, formyl, CONH₂, CSNH₂, CH═N—OH, CH═N—O—(C₁-C₆)-alkyl, C(═O)R^{7c}, C(═S)R^{7c}, C₁-C₆-alkyl, C₂-C₆-alkenyl, C₂-C₆-alkynyl, C₃-C₈-cycloalkyl, C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkylthio, C₂-C₆-alkenylthio, C₂-C₆-alkynylthio, C₁-C₆-alkylsulfonyl, C₂-C₆-alkenylsulfonyl, C₂-C₆-alkynylsulfonyl, (C₁-C₆-alkyl)carbonyl, (C₂-C₆-alkenyl)-carbonyl, (C₂-C₆-alkynyl)-carbonyl, C₁-C₆-alkoxy, C₂-C₆-alkenyloxy, C₂-C₆-alkynyloxy, (C₁-C₆-alkoxy)carbonyl, (C₂-C₆-alkenyloxy)carbonyl, (C₂-C₆-alkynyloxy)-carbonyl, (C₁-C₆-alkyl)carbonyloxy, (C₂-C₆-alkenyl-)carbonyl-oxy, (C₂-C₆-alkynyl)carbonyloxy, (C₁-C₆-alkyl)carbonyl-amino, (C₂-C₆-alkenyl)carbonyl-amino, (C₂-C₆-alkynyl)carbonyl-amino, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio; C(O)NR^{7a}R^{7b} or (SO₂)NR^{7a}R^{7b}; wherein R^{7a}, R^{7b} and R^{7c} are defined as below,
a radical Y—Ar or a radical Y-Cy, wherein
Y is a single bond, O, S, NH, C₁-C₆-alkandiyl or C₁-C₆-alkandiyloxy;
Ar is phenyl, naphthyl or a mono- or bicyclic 5- to 10-membered heteroaromatic ring, which contains 1, 2, 3 or 4 heteroatoms selected from 1 or 2 oxygen, 1 or 2 sulfur and 1 to 3 nitrogen atoms as ring members, wherein Ar is unsubstituted or may carry any combination of one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
Cy is C₃-C₁₂-cycloalkyl, which is unsubstituted or substituted with one to five radicals, independently of one another selected from the group consisting of halogen, cyano, nitro, hydroxy, mercapto, amino, carboxyl, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio;
and wherein the radical R⁷ that is bound to adjacent carbon atoms of the phenyl rings may optionally form, together with said carbon atoms, a fused benzene ring, a fused saturated or partially unsaturated 5-, 6-, or 7-membered carbocycle or a fused 5-, 6- or 7-membered heterocycle, which contains 1, 2, 3 or 4 heteroatoms selected from 1 or 2 oxygen, 1 or 2 sulfur and 1 to 3 nitrogen atoms as ring members, and wherein the fused ring is unsubstituted or may carry 1, 2, 3 or 4 radicals, independently of one another selected from the group consisting of halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy and C₁-C₆-alkylthio;
and wherein
R^{7a} and R^{7b} are each independently selected from one another from hydrogen, C₁-C₆-alkyl, C₁-C₆-haloalkyl, C₂-C₆-alkenyl, or C₂-C₆-alkynyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio;
R^{7c} in each instance is selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl or heteroaryl, which can be a mono- or bicyclic 5 to 10 membered heteroaromatic ring, which contains 1, 2, 3 or 4 heteroatoms selected from O, S and N.

3. An 1-(Azolin-2-yl)-amino-1-phenyl-2-hetaryl-ethane compound according to claim 1, wherein W is selected from: 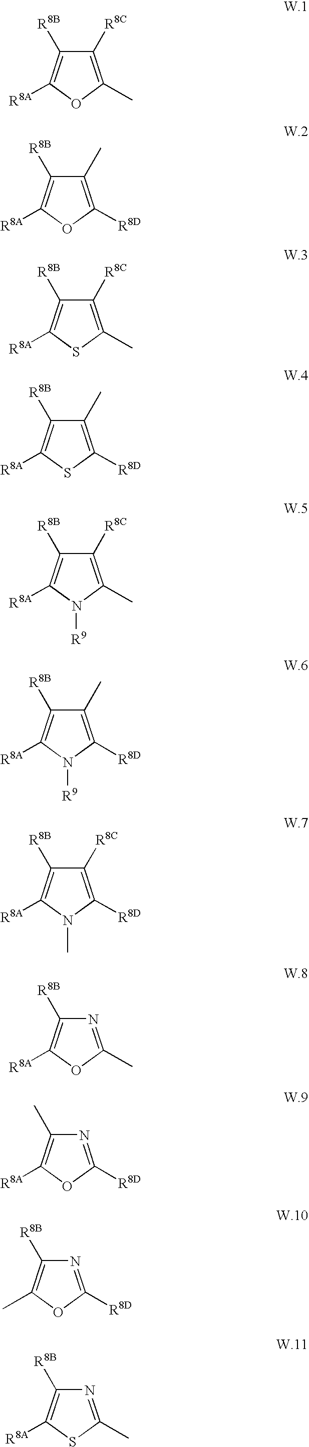 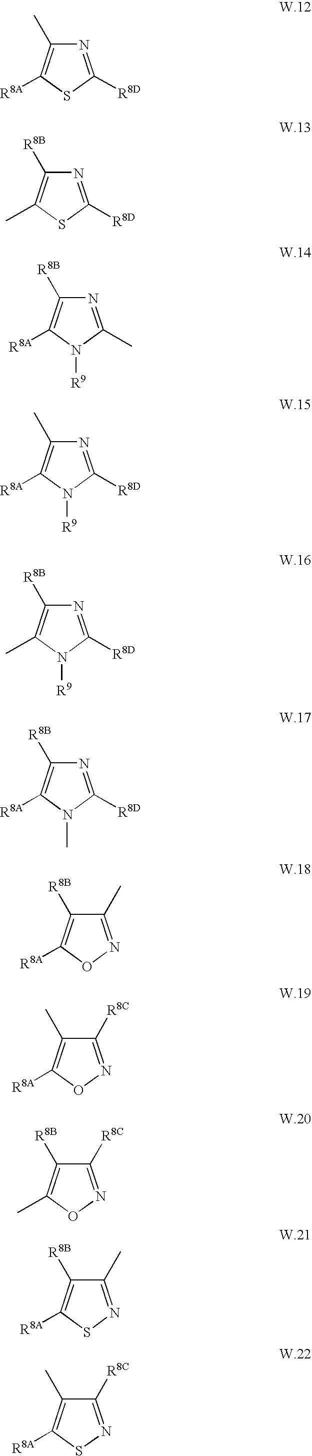 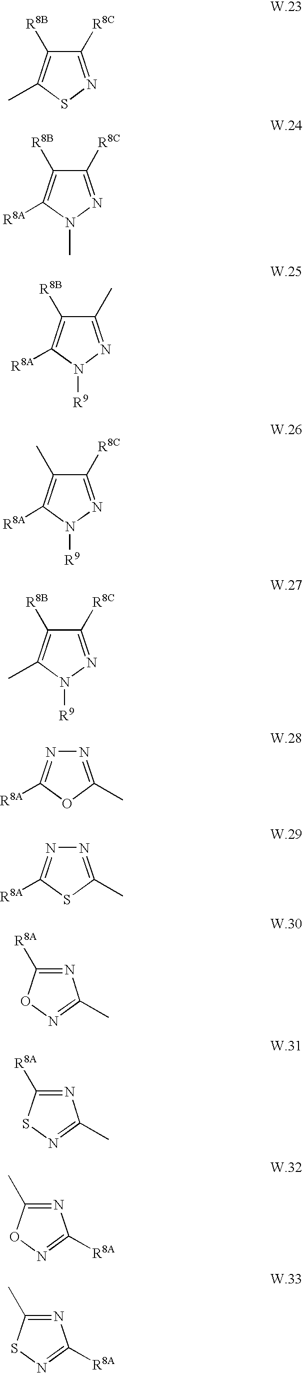 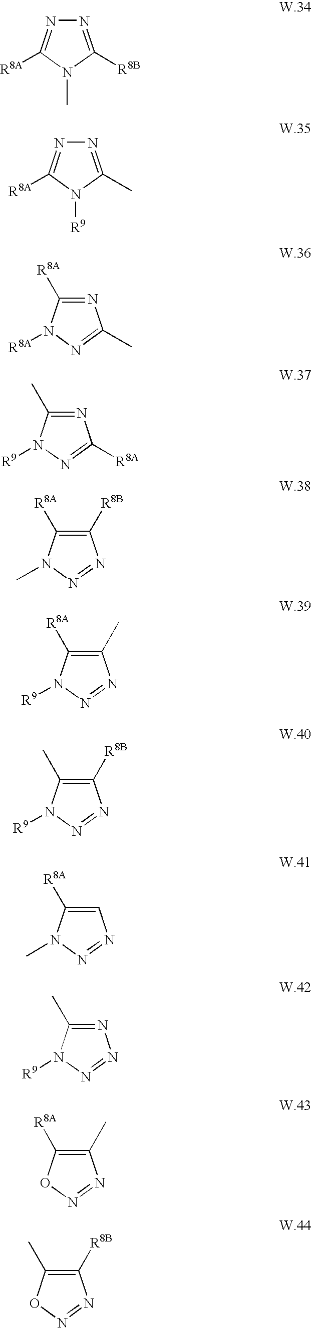 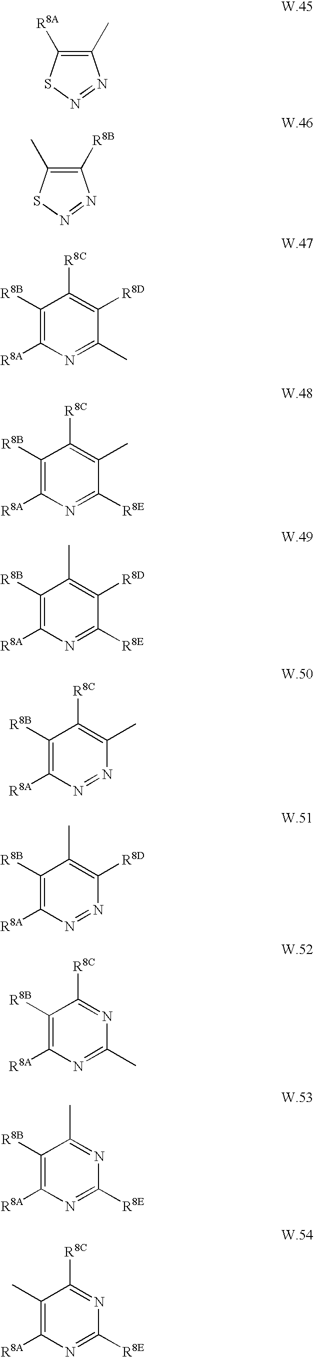 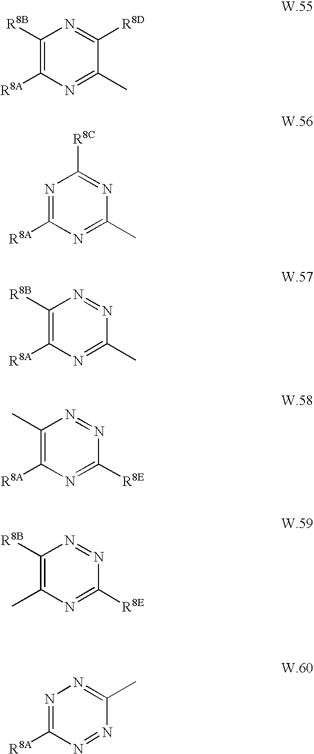
wherein the free bond denotes the position of attachment in formula I, wherein the capital letters A, B, C, D and E joined to R⁸ indicate the position of R⁸ at the heteroaromatic ring system and R⁸ is defined as in claim 1.

4. An 1-(Azolin-2-yl)-amino-1-phenyl-2-hetaryl-ethane compound according to claim 3, wherein W is selected from formulas W.1, W.2, W.3, W.4, W.5, W.6, W.8, W.9, W.10, W.11, W.12, W.13, W.14, W.15, W.17, W.18, W.19, W.20, W.21, W.22, W.23, W.25, W.26, W.27, W.28, W.29, W.30, W.31, W.32, W.33, W.47, W.48, W.49, W.52, W.53 and W.54.

5. An 1-(Azolin-2-yl)-amino-1-phenyl-2-hetaryl-ethane compound according to claim 3, wherein W is selected from formulas W.1, W.3, W.25, W.26, W.27, W.47, W.48, W.49, W.52, W.53 and W.54.

6. An 1-(Azolin-2-yl)-amino-1-phenyl-2-hetaryl-ethane compound according to claim 1, wherein
R¹, R², R³ are selected from hydrogen, C₁-C₆-alkyl, C₁-C₆-haloalkyl or C₃-C₆-cycloalkyl, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy, C₁-C₆-haloalkyl and C₁-C₆-alkylthio.

7. An 1-(Azolin-2-yl)-amino-1-phenyl-2-hetaryl-ethane compound according to claim 1, wherein R¹, R² and R³ are selected from hydrogen and C₁-C₆-alkyl.

8. An 1-(Azolin-2-yl)-amino-1-phenyl-2-hetaryl-ethane compound according to claim 1, wherein R¹, R² and R³ are hydrogen.

9. An 1-(Azolin-2-yl)-amino-1-phenyl-2-hetaryl-ethane compound according to claim 1, wherein
R^{4a}, R^{4b}, R^{4c}, R^{4d}, are each independently selected from one another from hydrogen, halogen, C₁-C₆-alkyl or C₁-C₆-haloalkyl

10. An 1-(Azolin-2-yl)-amino-1-phenyl-2-hetaryl-ethane compound according to claim 1, wherein R^{4a}, R^{4b}, R^{4c} and R^{4d} are hydrogen.

11. An 1-(Azolin-2-yl)-amino-1-phenyl-2-hetaryl-ethane compound according to claim 1, wherein
R⁵, R⁶, R⁹ are each independently selected from one another from hydrogen, cyano, nitro, formyl, C(═O)R^{5c}, C(═O)R^{6c}, C(═O)R^{9c}, C₁-C₆-alkyl, C₂-C₆-alkinyl, C₃-C₈-cycloalkyl, (C₁-C₆-alkoxy)methylen, C₁-C₆-alkylsulfinyl, C₁-C₆-alkylsulfenyl or C₁-C₆-alkylsulfonyl wherein the carbon atoms in the aliphatic radicals of the aforementioned groups may carry any combination of 1, 2 or 3 radicals, independently of one another selected from the group consisting of halogen, cyano, nitro, hydroxy, mercapto, amino, carboxyl, C₁-C₆-alkyl, C₁-C₆-alkoxy, C₂-C₆-alkenyloxy, C₂-C₆-alkynyloxy, C₁-C₆-haloalkoxy and C₁-C₆-alkylthio and wherein
R^{5c}, R^{6c} and R^{9c} are selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl or heteroaryl, which can be a mono- or bicyclic 5 to 10 membered heteroaromatic ring, which contains 1, 2, 3 or 4 heteroatoms selected from O, S and N.

12. An 1-(Azolin-2-yl)-amino-1-phenyl-2-hetaryl-ethane compound according to claim 1, wherein R⁵, R⁶, R⁹ are each independently selected from one another from hydrogen, cyano, nitro, formyl, C(═O)R^{5c}, C(═O)R^{6c}, C(═O)R^{9c} or C₁-C₆-alkyl and wherein
R^{5c}, R^{6c} and R^{9c} are selected each independently from one another from hydrogen, C₁-C₆ alkyl, C₂-C₆-alkenyl, C₂-C₆-alkinyl, C₃-C₈-cycloalkyl, C₁-C₆-alkylthio, C₁-C₆-alkoxy, (C₁-C₆-alkyl)amino, di(C₁-C₆-alkyl)amino, hydrazino, (C₁-C₆-alkyl)hydrazino, di(C₁-C₆-alkyl)hydrazino, phenyl and heteroaryl, which can be a mono- or bicyclic 5 to 10 membered heteroaromatic ring, which contains 1,2, 3 or 4 heteroatoms selected from O, S and N

13. An 1-(Azolin-2-yl)-amino-1-phenyl-2-hetaryl-ethane compound according to claim 1, wherein R⁵, R⁶ and R⁹ are selected from hydrogen.

14. An 1-(Azolin-2-yl)-amino-1-phenyl-2-hetaryl-ethane compound according to claim 1, wherein
R⁷, R⁸ are both independently from one another selected from halogen, OH, SH, NH₂, SO₃H, COOH, cyano, CONH₂, C(═O)R^{7c}, C(═O)R^{8c}, C₁-C₆-alkyl, C₃-C₈-cycloalkyl, C₁-C₆-alkylamino, or di(C₁-C₆-alkyl)amino, wherein the carbon atoms in the aliphatic radicals of the aforementioned groups may carry any combination of one, two or three radicals, independently of one another selected from the group consisting of halogen, cyano, nitro, hydroxy, mercapto, amino, carboxyl, C₁-C₆-alkyl, C₂-C₆-alkenyl, C₂-C₆-alkynyl, C₁-C₆-alkoxy, C₂-C₆-alkenyloxy, C₂-C₆-alkynyloxy, C₁-C₆-haloalkoxy and C₁-C₆-haloalkyl; and wherein
R^{7c}, R^{8c} are both selected each independently from one another from hydrogen, C₁-C₆-alkyl, C₂-C₆-alkenyl, C₂-C₆-alkinyl, C₃-C₈-cycloalkyl, C₁-C₆-alkoxy, (C₁-C₆-alkyl)amino, di(C₁-C₆-alkyl)amino, phenyl and heteroaryl, which can be a mono- or bicyclic 5 to 10 membered heteroaromatic ring, which contains 1, 2, 3 or 4 heteroatoms selected from O, S and N.

15. An 1-(Azolin-2-yl)-amino-1-phenyl-2-hetaryl-ethane compound according to claim 1, wherein
R⁷, R⁸ are both selected each independently from one another from halogen, cyano, C₁-C₆-alkyl or C₁-C₆-alkoxy, and wherein the radicals may be unsubstituted, partially or fully halogenated.

16. An 1-(Azolin-2-yl)-amino-1-phenyl-2-hetaryl-ethane compound according to claim 1, wherein X is sulfur.

17. An 1-(Azolin-2-yl)-amino-1-phenyl-2-hetaryl-ethane compound according to claim 1, wherein m is 0, 1, 2 or 3.

18. An 1-(Azolin-2-yl)-amino-1-phenyl-2-hetaryl-ethane compound according to claim 1, wherein m is 0, 1 or 2.

19. An 1-(Azolin-2-yl)-amino-1-phenyl-2-hetaryl-ethane compound according to claim 1, wherein n is 0, 1, 2 or 3.

20. An 1-(Azolin-2-yl)-amino-1-phenyl-2-hetaryl-ethane compound according to claim 1, wherein n is 0, 1 or 2.

21. A composition comprising at least one 1-(Azolin-2-yl)-amino-1-phenyl-2-hetaryl-ethane compound of formula I as defined in claim 1 or an enantiomer, diastereomer and/or salt thereof and at least one inert liquid and/or solid carrier.

22. An agricultural or veterinary composition comprising at least one 1-(Azolin-2-yl)-amino-1-phenyl-2-hetaryl-ethane compound of formula I as defined in claim 1 or an enantiomer, diastereomer and/or agriculturally or veterinary useful salt thereof and at least one inert liquid and/or solid agronomically acceptable carrier and, if desired, at least one surfactant.

23. (canceled)

24. (canceled)

25. A method of combating animal pests which comprises contacting the animal pests, their habit, breeding ground, food supply, plant, seed, soil, area, material or environment in which the animal pests live or grow or may live or grow or the materials, plants, seeds, soils, surfaces or spaces to be protected from animal attack or infestation with a pesticidal effective amount of at least one 1-(Azolin-2-yl)-amino-1-phenyl-2-hetaryl-ethane compound of formula I as defined in claim 1 or an enantiomer, diastereomer or an agricultural or veterinary acceptable salt thereof.

26. The method as defined in claim 25, wherein the animal pests are insects, arachnids or nematodes.

27. The method as defined in claim 25, wherein the animal pest are insects like Homoptera, Lepidoptera or Coleoptera or arachnids of the order of Acarina.

28. A method for protecting crops from attack or infestation by animal pests, which comprises contacting a crop with a pesticidal effective amount of at least one 1-(Azolin-2-yl)-amino-1-phenyl-2-hetaryl-ethane compound of formula I as defined in claim 1 or an enantiomer, diastereomer or an agricultural acceptable salt thereof.

29. A method for the protection of seeds from soil insects and of the seedlings' roots and shoots from insects comprising contacting the seeds before sowing and/or after pregermination with an 1-(Azolin-2-yl)-amino-1-phenyl-2-hetaryl-ethane compound of formula I as defined in claim 1 or an enantiomer, diastereomer and/or an agriculturally acceptable salt thereof, in pesticidal effective amounts.

30. The method according claim to 29, wherein the 1-(Azolin-2-yl)-amino-1-phenyl-2-hetaryl-ethane compound is applied in an amount of from 0.1 g to 10 kg per 100 kg of seeds.

31. A method according to claim 29, wherein of the resulting plant's roots and shoots are protected.

32. A method according to claim 29, wherein the resulting plant's shoots are protected from aphids.

33. Seed comprising an 1-(Azolin-2-yl)-amino-1-phenyl-2-hetaryl-ethane compound of the formula I as defined in claim 1 or an enantiomer, diastereomer and/or an agriculturally acceptable salt thereof.

34. Seed of claim 33 wherein, said compound, enantiomer, diastereomer and/or an agriculturally acceptable salt thereof is present in an amount of from 0.1 g to 10 kg per 100 kg of seed.

35. (canceled)

36. A method for treating, controlling, preventing or protecting animals against infestation or infection by parasites by administering to the animals a parasitically effective amount of an 1-(Azolin-2-yl)-amino-1-phenyl-2-hetaryl-ethane compound of formula I as defined in claim 1 or an enantiomer, diasteromer and/or an veterinary acceptable salt thereof.

37. A method according to claim 36 wherein said administering comprises orally, topically or parenterally administering or applying.

38. (canceled)
